SEQUENCE LISTING (22 sequences)

SEQ ID NO: 1 - DNA, 1884 bp, African swine fever virus
  CDS (1)..(1881)
atggactacaaggacgatgacgataaggcagggccaggacctggcccctcagattttattctcaatatcagcatgaagatggaagtcatttttaagaccgacctccgctcctcctcccaggtggtgtttcacgctggaagcctgtacaactggttctccgtcgagatcattaattctgggcgcatcgtgaccacagccattaagaccctgctcagcacagtgaagtacgacattgtcaaatccgcccggatctacgctggccaggggtataccgagcaccaggcccaggaggaatggaacatgatcctgcatgtgctctttgaggaagagaccgagagcacaagctccgcctctagcgagtccatccatgaaaagaacgaccacgaaaagaatggccataaagctgacgataacgagaccaatgaatgcacatcctctttcgagaccctgtttgagcaggaaccaagctccaccgaaacacccaaggacagcaaactgtacatgctcgcccagaagaccgtccagcacattgagcagtatggcaaggccccagatttcaacaaagtgatcagggctcacaattttatccagaccattcatgggacacccctgaaggaagaggaaaaagaggtggtcagactgatggtcatcaagctgctgaagaagatcagcttctacctgacctatatcaacaagctgctctcccatctgcacctcatgtttggcggaggaggcagcggaggaggagggtccggaggaggaggatctatgaggtctagcaagaaaatcaacaataagaaaaacatgttcaacatcaagatgtctaccattagcacactgctcatcgcctttctgtactataagaaacagcagccccctaagaaagtgtgcaaggtggacaaagattgcggaagcggagagcactgcgtcaggggcagctgttcctctctgtcctgcctcgacgccgtgaagatggataagagaaacatcaagatcgactccaaaatcagctcctgtgagttcacccccaatttctaccgctttaccgacacagccgctgatgagcagcaggaatttggcaagacccggcaccctatcaaaattacaccaagcccctccgagtctcatagcccacaggaggtgtgcgaaagatactgtagctggggcaccgacgattgtacagggtgggagtatgtgggagacgagaaggaaggcacctgctacgtgtataacaatccccaccatcctgtcctgaagtacggcaaagatcacgtgatcgccctgccaaggaaccataagcacgctggaggaggaggctctggaggaggagggagcggaggaggaggcagcatggacaccgagacatcccctctgctctctcacaacctgagcacccgcgaggggattaagcagtccacccagggactgctcgcccacacaatcgctcgctaccccggcaccacagccatcctgctcgggattctgtataacagaagcgtggattgcaagtctagcatgccaaaaccacccccttcctactatgtccagcagcctgagccacaccatcacttccccgtgttctttcgcaagcggaaaaactccacctctcagcagtcccacatcccttctgacgagcagctggctgaactcgctcactccggcggcggcggcagcggcggcggagggtccggaggcgggggatctatggctctggatggctcctctggaggaggcagcaacgtggagaccctgctcatcgtcgccttctggtggatgccccggcagcagcagaagaaatgtagcaaggctgaggaatgcacctgtaacaatggcagctgctccctgaagacatcctacccatacgatgttccagattacgcttga

SEQ ID NO: 2 - protein, 627 aa, African swine fever virus
MDYKDDDDKAGPGPGPSDFILNISMKMEVIFKTDLRSSSQVVFHAGSLYNWFSVEIINSGRIVTTAIKTLLSTVKYDIVKSARIYAGQGYTEHQAQEEWNMILHVLFEEETESTSSASSESIHEKNDHEKNGHKADDNETNECTSSFETLFEQEPSSTETPKDSKLYMLAQKTVQHIEQYGKAPDFNKVIRAHNFIQTIHGTPLKEEEKEVVRLMVIKLLKKISFYLTYINKLLSHLHLMFGGGGSGGGGSGGGGSMRSSKKINNKKNMFNIKMSTISTLLIAFLYYKKQQPPKKVCKVDKDCGSGEHCVRGSCSSLSCLDAVKMDKRNIKIDSKISSCEFTPNFYRFTDTAADEQQEFGKTRHPIKITPSPSESHSPQEVCERYCSWGTDDCTGWEYVGDEKEGTCYVYNNPHHPVLKYGKDHVIALPRNHKHAGGGGSGGGGSGGGGSMDTETSPLLSHNLSTREGIKQSTQGLLAHTIARYPGTTAILLGILYNRSVDCKSSMPKPPPSYYVQQPEPHHHFPVFFRKRKNSTSQQSHIPSDEQLAELAHSGGGGSGGGGSGGGGSMALDGSSGGGSNVETLLIVAFWWMPRQQQKKCSKAEECTCNNGSCSLKTSYPYDVPDYA

SEQ ID NO: 3 - DNA, 675 bp, African swine fever virus
  CDS (1)..(672)
atggactacaaggacgatgacgataaggcagggccaggacctggcccctcagattccgagttctttcagcccgtctacattcagcagtacgtcgagtgcctctctcctgtgaccccaccttccttttcttacgcctacaggtgcttctttgtggtcaacttcgtggtcctgaccgccgacatcgtgctgcgcagctccctctgtgcctcccggaagaaaaaggccgctgccgctattgccgaggaagacatccagtttattaacccatatcaggatcagcagtgggctgaggtgtcccagcagcccggcaccagcaaacctgctggagctaccacagctagcgccggaaagccagtgaccggcaggcccgctacaaacagacctgccaccaataagccagtgatggtcacagacaaccccgtgaccgataggctggtcatggctaccggaggaccagctgctaccaatacagccgctcctgccgctgcctctgctgccgctagcgccgctgcctccgtgcccgctcaccctaccgaaccatacaccacagtcaccacacagaacaccgcctctcagacaatgagcgccatcgagaacctccgccagcggaacacctacacacataaggatctggaaaattccctctacccatacgatgttccagattacgcttga

SEQ ID NO: 4 - protein, 224 aa, African swine fever virus
MDYKDDDDKAGPGPGPSDSEFFQPVYIQQYVECLSPVTPPSFSYAYRCFFVVNFVVLTADIVLRSSLCASRKKKAAAAIAEEDIQFINPYQDQQWAEVSQQPGTSKPAGATTASAGKPVTGRPATNRPATNKPVMVTDNPVTDRLVMATGGPAATNTAAPAAASAAASAAASVPAHPTEPYTTVTTQNTASQTMSAIENLRQRNTYTHKDLENSLYPYDVPDYA

SEQ ID NO: 5 - DNA, 1668 bp, African swine fever virus
  CDS (1)..(1665)
atggactacaaggacgatgacgataaggcagggccaggacctggcccctcaccctccaacatgaagcagttctgtaaaatctctgtctggctccagcagcacgaccccgacctcctcgaaattatcaacaatctctgcatgctcggaaatctgagcgccgctaagtacaaacacggcgtgaccttcatctatcccaagcaggccaaaatccgggacgagattaagaaacatgcctactctaacgatcccagccaggctatcaagaccctggagtccctcatcctgcctttttacattcctacaccagctgagttcaccggagagattggctcttataccggggtgaaactggaggtcgaaaagacagaagccaacaaagtgatcctgaagaacggagaggctgtgctcgtccctgccgctgacttcaagccctttcctgatcgccggctggccgtgtggatcatggaatctgggagcatgccactggagggacccccttacaaaagaaagaaagagggaggaggaaacgacccacccgtgcccaagcacatctctccatatacccccaggacaagaatcgccattgaggtggaaaaggctttcgacgattgcatgcgccagaattggtgttccgtgaacaatccttacctggccaaatccgtctctctgctcagctttctctccctgaaccacccaaccgagttcatcaaggtgctccccctgattgacttcgatcctctggtcaccttttacctgctcctggaaccctataagacacacggcgacgattttctgatccctgagaccattctcttcgggccaacaggatggaacggcaccgacctgtaccagagcgccatgctggagttcaagaaattctttacccagattacacggcagaccttcatggacatcgccgattccgctaccaaagaagtggacgtgccaatttgctactccgatcccgagacagtgcactcttataccaatcatgtcaggacagagatcctgcaccataacgccgtgaataaggtcaccacacccaacctggtggtccaggcttacaatgagctggaacagaccaacacaatcagacactatggccctatttttccagaaagcaccatcaacgccctgcgcttctggaagaaactctggcaggacgagcagcggttcgtgatccacgggctgcataggaccctcatggatcagcctacatacgagacctctgaatttgccgagatcgtgcgcaacctgcggttcagcaggccaggaaacaattacatcaacgagctgaatattaccagccccgccatgtatggcgacaagcacaccacaggggacatcgctccaaatgatcggttcgccatgctggtggcttttattaactccaccgacttcctgtatacagccatccccgaggaaaaagtgggcgggaacgagacccagacaagctccctcaccgatctggtccctacaaggctgcacagcttcctcaaccataatctctccaagctgaaaattctcaatagagcccagcagaccgtgcgcaacatcctgtccaatgactgtctgaaccagctcaagcactacgtcaaacataccgggaagaacgaaatcctgaaactcctgcaggagtacccatacgatgttccagattacgcttga

SEQ ID NO: 6 - protein, 555 aa, African swine fever virus
MDYKDDDDKAGPGPGPSPSNMKQFCKISVWLQQHDPDLLEIINNLCMLGNLSAAKYKHGVTFIYPKQAKIRDEIKKHAYSNDPSQAIKTLESLILPFYIPTPAEFTGEIGSYTGVKLEVEKTEANKVILKNGEAVLVPAADFKPFPDRRLAVWIMESGSMPLEGPPYKRKKEGGGNDPPVPKHISPYTPRTRIAIEVEKAFDDCMRQNWCSVNNPYLAKSVSLLSFLSLNHPTEFIKVLPLIDFDPLVTFYLLLEPYKTHGDDFLIPETILFGPTGWNGTDLYQSAMLEFKKFFTQITRQTFMDIADSATKEVDVPICYSDPETVHSYTNHVRTEILHHNAVNKVTTPNLVVQAYNELEQTNTIRHYGPIFPESTINALRFWKKLWQDEQRFVIHGLHRTLMDQPTYETSEFAEIVRNLRFSRPGNNYINELNITSPAMYGDKHTTGDIAPNDRFAMLVAFINSTDFLYTAIPEEKVGGNETQTSSLTDLVPTRLHSFLNHNLSKLKILNRAQQTVRNILSNDCLNQLKHYVKHTGKNEILKLLQEYPYDVPDYA

SEQ ID NO: 7 - DNA, 2016 bp, African swine fever virus
  CDS (1)..(2013)
atggactacaaggacgatgacgataaggcagggccaggacctggcccctcagcttctgggggggctttctgtctcatcgctaacgacggcaaggctgataaaatcattctcgctcaggacctcctcaactctcggatctccaacattaagaatgtgaacaaatcttacggcaagccagaccccgaacctaccctgagccagatcgaggaaacacacctcgtgcatttcaacgcccattttaaaccatacgtgcccgtcgggttcgagtataacaaggtgcgcccacacaccggcacacccaccctcgggaataagctgacctttggaatcccacagtacggcgacttctttcatgatatggtggggcaccatatcctgggagcttgccacagctcctggcaggacgctccaatccagggcaccagccagatgggagctcacggacagctgcagacattccctcggaacgggtacgactgggataatcagaccccactggaaggagccgtgtatacactcgtcgatcccttcggaaggcctatcgtgccaggcaccaagaacgcttacagaaatctggtctactattgcgagtaccccggagaaaggctctatgagaacgtgagattcgacgtgaatggcaactccctggacgagtactctagcgatgtgaccacactcgtccgcaagttttgtatccccggcgataaaatgaccgggtataagcacctggtgggccaggaagtgtctgtcgaggggaccagcggacctctgctctgcaacattcacgacctgcataaacctcaccagagcaagccaatcctcaccgacgaaaacgatacacagcggacctgttcccacacaaatcctaaattcctgtctcagcattttccagagaacagccacaatatccagaccgccgggaagcaggacatcacacccattaccgacgctacatacctggacatccgccggaacgtgcactatagctgtaatggaccccagacccctaaatactatcagccccctctcgccctgtggatcaagctgcgcttctggtttaatgaaaacgtgaatctcgctatcccctctgtcagcattcctttcggcgagcggtttatcaccatcaagctggcctcccagaaggacctcgtgaacgagttccccggcctgtttatcaggcagagccggttcattccagggcgcccctccaggagaaacatccggttcaagccctggtttatccccggcgtgatcaacgaaattagcctcaccaacaatgagctgtacatcaacaatctcttcgtgacacctgagattcacaacctgttcgtgaaacgcgtccggttttccctcatcagggtgcataagacccaggtcacacacaccaacaataaccaccatgacgaaaaactcatgtctgccctgaagtggcccatcgagtatatgttcattggcctgaaacccacctggaacatctccgaccagaatcctcaccagcatagggattggcataagttcgggcacgtggtcaacgccatcatgcagcctacccaccatgctgagatttcctttcaggacagagatacagccctgccagacgcttgctcctctatcagcgatatttccccagtgacctaccccatcacactgcctatcattaagaacatttccgtcaccgcccacggcatcaatctgattgacaaattcccatctaagttttgtagctcctacatccccttccactatggcgggaacgccatcaagacccctgacgatccaggggccatgatgatcacatttgctctgaagccaagggaggaataccagccctccggacacatcaacgtgtctagggccagagagttctacatttcttgggacaccgattatgtcggaagcatcaccacagctgacctggtggtctccgcctctgctatcaacttcctgctcctgcagaatggcagcgccgtgctgagatactccacctacccatacgatgttccagattacgcttga

SEQ ID NO: 8 - protein, 671 aa, African swine fever virus
MDYKDDDDKAGPGPGPSASGGAFCLIANDGKADKIILAQDLLNSRISNIKNVNKSYGKPDPEPTLSQIEETHLVHFNAHFKPYVPVGFEYNKVRPHTGTPTLGNKLTFGIPQYGDFFHDMVGHHILGACHSSWQDAPIQGTSQMGAHGQLQTFPRNGYDWDNQTPLEGAVYTLVDPFGRPIVPGTKNAYRNLVYYCEYPGERLYENVRFDVNGNSLDEYSSDVTTLVRKFCIPGDKMTGYKHLVGQEVSVEGTSGPLLCNIHDLHKPHQSKPILTDENDTQRTCSHTNPKFLSQHFPENSHNIQTAGKQDITPITDATYLDIRRNVHYSCNGPQTPKYYQPPLALWIKLRFWFNENVNLAIPSVSIPFGERFITIKLASQKDLVNEFPGLFIRQSRFIPGRPSRRNIRFKPWFIPGVINEISLTNNELYINNLFVTPEIHNLFVKRVRFSLIRVHKTQVTHTNNNHHDEKLMSALKWPIEYMFIGLKPTWNISDQNPHQHRDWHKFGHVVNAIMQPTHHAEISFQDRDTALPDACSSISDISPVTYPITLPIIKNISVTAHGINLIDKFPSKFCSSYIPFHYGGNAIKTPDDPGAMMITFALKPREEYQPSGHINVSRAREFYISWDTDYVGSITTADLVVSASAINFLLLQNGSAVLRYSTYPYDVPDYA

SEQ ID NO: 9 - DNA, 2760 bp, African swine fever virus
  CDS (1)..(2757)
atggactacaaggacgatgacgataaggcagggccaggacctggcccctcagggaacagaggctccagcacaagcagcagaccactcccaagcagcgaggctaacatctatgccaaactccaggatcacattcagaggcagaccaggcccttctctggaggaggatactttaatggcgggggagacaaaaaccctgtgcagcacattaaggactatcatatcgatagcgtcagctccaaggccaaactgaggatcattgaggggatcattagagctatcgccaagattggattcaaagtggataccaagcagcccatcgaggacattctgaaggacatcaagaagcagctccccgaccctcgggctggcagcaccttcgtgaaaaatgccgagaagcaggaaacagtctgcaaaatgatcgctgacgccattaaccaggagtttatcgacctgggccaggataagctcattgacaccacagagggggccgctagcatctgtaggcagattgtgctgtacatcaactccctgacccacggactcagagccgagtatctggatgtgcatggctccattgagaacacactggaaaatatcaaactgctcaacgacgccattaagcagctgcacgagaggatggtgaccgaagtcacaaaggccgctcctaatgaggaagtgattaacgctgtcaccatgatcgaggccgtgtaccgccggctgctcaacgaacagaatctgcagatcaacattctcaccaatttcatcgacaacattctgaccccaacacagaaagagctggataagctccagacagacgaagtggacatcatcaagctgctgaacgacaccaatagcgtgctggggacaaaaaatttcggaaaggtcctctcttacaccctgtgcaacctcggaatcgccgctagcgtggctaacaagatcaacaaggccctgcagaaagtgggcctcaaggtcgagcagtatctgcagtccaagaattgggccgagttcgataaagaactggacctcaagcggttcagcggactggtgagcgccgagaacatcgctgagttcgaaaaagccgtcaatctgctccgccagacctttaacgagcggcacaagatcctggaaaactcttgtgccaagaaaggcggggatgaggaaaagacccccctggacaggagaatcgaggcccagaggctcgatagaaaacatatcctgatggagttcctcaacaagtccacccaggcctacaatgactttctggagaacgtgaagaaaatcggcattaagctggtcaaagaaattgctctcacccctaatatcacaaggctgagagatgccctcagcaggattaacgacatggggaccatcgccctggatctctccctgatcggattctacacaaatgccgctgcccgcgaggaacgggagacctttctgacacagctcaccctggtgaagaacgtcctggaggaacagagcaaaaccgacccaaacttcaagaatctgtatgattcttgcagcagactgctccagatcattgatttttacaccgacatcgtgcagaagaaatatggaggcgaggaagactgcgagtgtacccgcgtgggaggagctgccctgacagtcgaggaactcggactgagcaaggctgctcggtcccaggtggacctgaatcaggccatcaacaccttcatgtactattactatgtggcccagatctactccaatctgacacacaacaagcaggagtttcagtcttacgaggaaaactatgccaccattctgggcgacgctatcgctgggcgcctgatgcagctcgatacagagaagaatgccaggatcaactcccctgctgtggacctggccagaggccatgtcggaccaaaccccggcggagctcaggaggtggactggaaagccaccgtctctgccatcgagctggaatacgatgtgaagcgccggttctatcgggccctggagggactcgacctgtacctcaaaaacatcaccaagacatttgtgaacaatatcgacagcattcagaccgtgcagcagatgctggatggagtccgcatcattggccggtggttcacagaggctaccggggacacactggcccaggtgttcgaatcctttccaacctctgccggcaacgattccaatgtcttcacagacaacgctcccgccgggcactactatgagaaggtggctgccgaaatccagcagggacggtccgtgggcaccctgaggcccgtcagagcttctcaggccaagaatattagggatctgatcgggagatccctctctaacttccaggccctgaagaacatcattaatgcttttgcccgcatcggagacatgctgggaggcgaggaactccggcagatggtgcctatgagcccactgcagatctacaaaaccctgctggagtatctgcagcacagcgccctctccgtgggcctgaagaacctcaatcagtccgagatcggaggacagagggtggctctggctcagaccgctgaggaagctgctcagcgcgtctacctgtctacagtgcgggtcaacgacgccctcagcaccagatgggagacagaagacgtgttcttcaccttcatgctgaaatccatggctgccaagatctttattgtgctggggatctacgacatgtttgaacgccctgagccagtctacaagctgattccaacacggatgattctcgggggagcctacccatacgatgttccagattacgcttga

SEQ ID NO: 10 - protein, 919 aa, African swine fever virus
MDYKDDDDKAGPGPGPSGNRGSSTSSRPLPSSEANIYAKLQDHIQRQTRPFSGGGYFNGGGDKNPVQHIKDYHIDSVSSKAKLRIIEGIIRAIAKIGFKVDTKQPIEDILKDIKKQLPDPRAGSTFVKNAEKQETVCKMIADAINQEFIDLGQDKLIDTTEGAASICRQIVLYINSLTHGLRAEYLDVHGSIENTLENIKLLNDAIKQLHERMVTEVTKAAPNEEVINAVTMIEAVYRRLLNEQNLQINILTNFIDNILTPTQKELDKLQTDEVDIIKLLNDTNSVLGTKNFGKVLSYTLCNLGIAASVANKINKALQKVGLKVEQYLQSKNWAEFDKELDLKRFSGLVSAENIAEFEKAVNLLRQTFNERHKILENSCAKKGGDEEKTPLDRRIEAQRLDRKHILMEFLNKSTQAYNDFLENVKKIGIKLVKEIALTPNITRLRDALSRINDMGTIALDLSLIGFYTNAAAREERETFLTQLTLVKNVLEEQSKTDPNFKNLYDSCSRLLQIIDFYTDIVQKKYGGEEDCECTRVGGAALTVEELGLSKAARSQVDLNQAINTFMYYYYVAQIYSNLTHNKQEFQSYEENYATILGDAIAGRLMQLDTEKNARINSPAVDLARGHVGPNPGGAQEVDWKATVSAIELEYDVKRRFYRALEGLDLYLKNITKTFVNNIDSIQTVQQMLDGVRIIGRWFTEATGDTLAQVFESFPTSAGNDSNVFTDNAPAGHYYEKVAAEIQQGRSVGTLRPVRASQAKNIRDLIGRSLSNFQALKNIINAFARIGDMLGGEELRQMVPMSPLQIYKTLLEYLQHSALSVGLKNLNQSEIGGQRVALAQTAEEAAQRVYLSTVRVNDALSTRWETEDVFFTFMLKSMAAKIFIVLGIYDMFERPEPVYKLIPTRMILGGAYPYDVPDYA

SEQ ID NO: 11 - DNA, 2661 bp, African swine fever virus
  CDS (1)..(2658)
atggactacaaggacgatgacgataaggcagggccaggacctggcccctcagacgctctctccacccgctgggaaaccgaggatgtcttcttcaccttcatgctcaagtctatggctgctaaaatcttcattgtgctcggcatctacgacatgttcgaacgcccagagcccgtgtataagctgattccaaccaggatgatcctcggaggagctgatgagctggaacctgaggtcatcccagaagccgctgagctgtacttcaggctccctagactggccgaattttatcagaaactgttctcttttcgcgacgagaacgtgcagattagcatgctcccagaactggagggcatcttcagcgggctgatcaggatttttatgagacccatcgagctgatcaacattggcgactactccgaaaccgagatccggcagctgatcaaggaaattaatgtgatctaccagcacttcaacctggagtatggggaacaggaggccaccaagaaagctctgattcatttcgtgaatgagatcaaccgccggtttggagtcattaccaggacagaatgggagaagttccagaggatcgtgcaggaggccagaaccatgaacgactttggcatgatgaatcagacaaactactctatcctgcccgacgaggatgggtatacccagagctcccagctgctcccatccgatcgcttcatcagcccctccacccagcctacaccaaagtggaggcccgccctgtacaatatcgactctgtggatgtccagaccggcatgctgcagcctaacagccagtgggacctggtgcagaagttccgcaaacagctgagcgaaatgtttgaggaccccagcctgcagcaggaactggggaaggtcagctaccaggagctgattcggcaggccatcaacgaactgaagaaagagcacaccgacaagatccagatcgtgagcaaactgatccagggatctgagagcctcgccgacacagatgtgaacaagattttcctgtttcatgagaccgtcatcacaggactgaacctgctcagcgccatctacgtgctgctcaacaatttccggaacaatattaagggcctggacctcgataccatccagaaatccatcattgaatggctgcgcgagacacaggccgctaatgtgaaccgggctaacctcatcgactggctgggaaggaagcacggcgccattagcgaaatcagaaatcctggcctggtggtcaaagagaacgacgtgcgcctgagccgcgtgtaccccgatcctaccacaaatgccaccgctccacaggacaggaacctcaccacagagacactgttcgcttggattgtgccctacgtcggcatccccgctggaggaggggtgagaccagaacaggagctcgccgctaggtatctggtggacaaccagagaattatgcagctgctcctgaccaatatcttcgagatgacatctagctttaacaagatggtgcaggtcaggttccctgagaccagcacagctcaggtgcacctggactttaccggcctcatttccctgatcgactctctgatggccgatacaaagtacttcctgaatctcctgaggcctcacatcgataagaacatcatccagtactacgagaacagaagcaacccagggtccttttactggctggaggaacatctgattgacaagctcatcaaaccccctaccgatgctggcggccggcccctgcccggcggagaactgggactggagggcgtgaatcagatcattaacaagacctacacactcctgaccaaaccatataacgtcctgcagctcaggggaggagctcagaggagagacgctgctaacatccagattaacaataacccccagtcctctgaacggttcgagcagtacggccgcgtgttctcccggctcgtcttttatgacgctctggaaaataactctggactcagggtggagcaggtcgccctgggggattttcgcctcagcaacctgatccggaccaataacgcccaggaggaaaatacactgtcctactgggacaacatggccctgagaacctatgctaatgtgaacgatgccgctaataacctccgccggtacaggctgtatgggtccgactacggaatccagaataacagatctatgatgatggtgttcaaccagctggtcgccagctacatcgctcgcttttatgatgctccctccggcaagatctacctgaatctcatcaacgccttcgctaatggaaactttagccaggccgtgatggagctgggatatgctcacccagacctcgctagggataataacgccttcgggcatagaggagaccctaccgagcagtctgtgctcctgctcagcctggggctcatgctgcagcgcctgatcaaggataccaaccggcagggactgtcccagcacctcatctctaccctgacagaaatccctatctacctgaaggagaattatcgggccaacctcccactgttcaacaagatgtttaacatcctgatttcccagggcgagctgctcaagcagttcatccagtacaccaatgtccagctcgccagacctaacctcacagggctcctcggggctaacaatgactccgtgtacccatacgatgttccagattacgcttga

SEQ ID NO: 12 - protein, 886 aa, African swine fever virus
MDYKDDDDKAGPGPGPSDALSTRWETEDVFFTFMLKSMAAKIFIVLGIYDMFERPEPVYKLIPTRMILGGADELEPEVIPEAAELYFRLPRLAEFYQKLFSFRDENVQISMLPELEGIFSGLIRIFMRPIELINIGDYSETEIRQLIKEINVIYQHFNLEYGEQEATKKALIHFVNEINRRFGVITRTEWEKFQRIVQEARTMNDFGMMNQTNYSILPDEDGYTQSSQLLPSDRFISPSTQPTPKWRPALYNIDSVDVQTGMLQPNSQWDLVQKFRKQLSEMFEDPSLQQELGKVSYQELIRQAINELKKEHTDKIQIVSKLIQGSESLADTDVNKIFLFHETVITGLNLLSAIYVLLNNFRNNIKGLDLDTIQKSIIEWLRETQAANVNRANLIDWLGRKHGAISEIRNPGLVVKENDVRLSRVYPDPTTNATAPQDRNLTTETLFAWIVPYVGIPAGGGVRPEQELAARYLVDNQRIMQLLLTNIFEMTSSFNKMVQVRFPETSTAQVHLDFTGLISLIDSLMADTKYFLNLLRPHIDKNIIQYYENRSNPGSFYWLEEHLIDKLIKPPTDAGGRPLPGGELGLEGVNQIINKTYTLLTKPYNVLQLRGGAQRRDAANIQINNNPQSSERFEQYGRVFSRLVFYDALENNSGLRVEQVALGDFRLSNLIRTNNAQEENTLSYWDNMALRTYANVNDAANNLRRYRLYGSDYGIQNNRSMMMVFNQLVASYIARFYDAPSGKIYLNLINAFANGNFSQAVMELGYAHPDLARDNNAFGHRGDPTEQSVLLLSLGLMLQRLIKDTNRQGLSQHLISTLTEIPIYLKENYRANLPLFNKMFNILISQGELLKQFIQYTNVQLARPNLTGLLGANNDSVYPYDVPDYA

SEQ ID NO: 13 - DNA, 2586 bp, African swine fever virus
  CDS (1)..(2583)
atggactacaaggacgatgacgataaggcagggccaggacctggcccctcactcaccgaaatccccatctacctcaaagaaaactaccgggccaacctccctctcttcaacaaaatgttcaatatcctcatctcccagggcgagctgctcaagcagttcatccagtacacaaacgtgcagctggccaggcctaatctcaccgggctgctcggagctaacaatgacagcgtcatctactataacaataacattaacgtgccaatgaccggactgtccgtcggacaggccgctctcaggggaatcggaggggtgttcagacctaacgtcacactgatgccactcggcgacgcccagaataacaccagcgatgtggtcaggaagagactggtggccgtcatcgacggcatcattagggggagccacaccctggctgattccgctatggaggtgctgcacgaactcaccgaccatcccatctacctggagacagaggaacacttcatccagaactatatgtcccggtacaataaggagcctctgatgccattttctctgagcctctactatctgcgcgacctccggatcgagaataacgaagtgtatgatcccctgctctaccctaacctggagtccggctctcctgagttcaagctgctctacggaacccgcaaactgctcggcaacgacccagtgcagctgagcgatatgcctggcgtccagctcatcatgaaaaattataacgagacagtggtcgcccgcgaacagatcacccccacacggttcgagcacttttacacccatgccatccaggctctgaggttcatcgtgaacattcgcagcttcaagaccgtcatgatgtacaatgagaacacattcggaggcgtgaacctgatctccgaaaatagggacgataaaccaatcattacagccgggatcggaatgaacgccgtgtactccctgagaaagaccctccaggacgtgatctcttttgtcgagagctcctaccaggaggaacagatcaatcacattcataagatcgtgagccccaaagggcagaccaggacactgggcagcaacagggagagggaaaggattttcaacctgttcgatatgaacatcatccccatcaatgtgaacgccctgatgagatccatccctctcgctaatatctacaactatgactactctttcgaggaaatcgcctgcctgatgtacggcatttctgctgagaaggtgcgcagcctgaacaccgctgctccacagcctgacatcgctgaggtcctgaatattcccaaccgcccccctatgaatacccgcgagttcatgctgaaactgctcatcaacccttatgtgagcgtctccattacccagtacgggaacgagctgctcagcaagggcaatgccgggtacatgtccaggatcttcagaggcgataatgccctgaacatgggcaggcccaaatttctctctgaccagatcttcaacaaggtgctgtttggcagcctctatcctacccagttcgactacgatgaggctggaccatctctggctgctggaatccagagaggccgcgagcggtggggacaccccatgtccatctacattaaccaggccctgcatgagctcgtgcgcaccatccggctggctgaaacagtgagggggctcagaaacgtcatcgataagaatcagctgattggagagctcaacgccttcaggacccagctggaggacacacgccgggaagtgaataacctgatccagacccccgagattcagaataacccaacacccgaaatcattgccgctatccagaactgggtgcagcagtacagaggccagatcaccaacctgattgatctcatcggaaatgccggccaggctaattccatgattaacctgatccagaatattacccctcagacagccggagctcagctgaccgccctcttcaacatcaggggactgccagctccaccacctaggcagatcctgcagaatgacattgaggccatgcagtggtttatgaccatggtcatcaaccacccacccatcctgattgcccccttcatgctgctcgtgaataacctgaaagagtttctgaacaccctcgaaaggtatgtctacaagacaccaagatggctgggccccggcaccgctaggatcgctcagcctccagtgggaatggcccctggaatcaacatgcggcaccatacctcctatacagagaacagcgtgctgacctacatcacagaacagaaccgcgaggaagggccatggagcatcgtgaagcaggtcggagtcggcattcagaaacccaccctggtccagatcggaaaggaccggttcgatacacgcctgattcggaacctcatctttattaccaatatccagaggctgctcaggctgagactcaacctggagctctctcagttcagaaatgtgctggtcagccccgatcacatcattaacccttccatcaccgagtatggcttctctattaccgggccatctgagaccttcagcgacaaacagtacgactccgacatccggattctgtacccatacgatgttccagattacgcttga

SEQ ID NO: 14 - protein, 861 aa, African swine fever virus
MDYKDDDDKAGPGPGPSLTEIPIYLKENYRANLPLFNKMFNILISQGELLKQFIQYTNVQLARPNLTGLLGANNDSVIYYNNNINVPMTGLSVGQAALRGIGGVFRPNVTLMPLGDAQNNTSDVVRKRLVAVIDGIIRGSHTLADSAMEVLHELTDHPIYLETEEHFIQNYMSRYNKEPLMPFSLSLYYLRDLRIENNEVYDPLLYPNLESGSPEFKLLYGTRKLLGNDPVQLSDMPGVQLIMKNYNETVVAREQITPTRFEHFYTHAIQALRFIVNIRSFKTVMMYNENTFGGVNLISENRDDKPIITAGIGMNAVYSLRKTLQDVISFVESSYQEEQINHIHKIVSPKGQTRTLGSNRERERIFNLFDMNIIPINVNALMRSIPLANIYNYDYSFEEIACLMYGISAEKVRSLNTAAPQPDIAEVLNIPNRPPMNTREFMLKLLINPYVSVSITQYGNELLSKGNAGYMSRIFRGDNALNMGRPKFLSDQIFNKVLFGSLYPTQFDYDEAGPSLAAGIQRGRERWGHPMSIYINQALHELVRTIRLAETVRGLRNVIDKNQLIGELNAFRTQLEDTRREVNNLIQTPEIQNNPTPEIIAAIQNWVQQYRGQITNLIDLIGNAGQANSMINLIQNITPQTAGAQLTALFNIRGLPAPPPRQILQNDIEAMQWFMTMVINHPPILIAPFMLLVNNLKEFLNTLERYVYKTPRWLGPGTARIAQPPVGMAPGINMRHHTSYTENSVLTYITEQNREEGPWSIVKQVGVGIQKPTLVQIGKDRFDTRLIRNLIFITNIQRLLRLRLNLELSQFRNVLVSPDHIINPSITEYGFSITGPSETFSDKQYDSDIRILYPYDVPDYA

SEQ ID NO: 15 - DNA, 552 bp, African swine fever virus
  CDS (1)..(543)
atgggggactacaaggacgatgacgataagaaaatgaacaagaagattatcgtgatgatggctctgctgcacaaagaaaaactgattgagtgtattgaaaatgaactggaaaacggaggcaccgtgctgctcctgacaaagaacatcgtggtctctgagatcagctacattggcaatacctacaaatatttcaccttcaacgacaatcacgatctcatctccaaggaagacctgaaaggggccacctctaacaatatcgctaagatgatctacaactggatcattaagaatccccagaacaacaaaatctggagcggagagcctcgcacccaaatctacttcgaaaacgacctctaccacacaaactacaaccatgagtgcatcaaagatttctggaacgtgagcacctccgtcggcccctgcatctttaacgatcggtccatttggtgtacaaagtgtacctccttttatccttttaccaacattatgagccccaacattttccagaaaaaatacccatacgacgttccggactacgcttcttagtgataa

SEQ ID NO: 16 - protein, 181 aa, African swine fever virus
MGDYKDDDDKKMNKKIIVMMALLHKEKLIECIENELENGGTVLLLTKNIVVSEISYIGNTYKYFTFNDNHDLISKEDLKGATSNNIAKMIYNWIIKNPQNNKIWSGEPRTQIYFENDLYHTNYNHECIKDFWNVSTSVGPCIFNDRSIWCTKCTSFYPFTNIMSPNIFQKKYPYDVPDYAS

SEQ ID NO: 17 - DNA, 435 bp, African swine fever virus
  CDS (1)..(426)
atgggggactacaaggacgatgacgataagaaaatgctccactgggggcctaaatactggcggtccctgcacctctacgctatcttcttctctgatgctccctcatggaaggaaaaatacgaggccatccagtggattctgaacttcatcgaatccctcccctgcacccgctgtcagcaccatgcttttagctacctgaccaagaacccactgacactcaacaattctgaggacttccagtattggacattcgcctttcacaacaatgtgaacaatcggctgaacaagaaaatcatctcttggtcagagtacaagaacatctatgaacagagcatcctgaagaccattgaatacggcaaaacagattttattggagcttggtccagcctcaaaaaatacccatacgacgttccggactacgcttcttagtgataa

SEQ ID NO: 18 - protein, 142 aa, African swine fever virus
MGDYKDDDDKKMLHWGPKYWRSLHLYAIFFSDAPSWKEKYEAIQWILNFIESLPCTRCQHHAFSYLTKNPLTLNNSEDFQYWTFAFHNNVNNRLNKKIISWSEYKNIYEQSILKTIEYGKTDFIGAWSSLKKYPYDVPDYAS

SEQ ID NO: 19 - DNA, 1683 bp, African swine fever virus
  CDS (1)..(1674)
atgggggactacaaggacgatgacgataagaaagccgaattcaatatcgacgaactcctcaaaaatgtcctggaagacccaagcacagagattagcgaagagaccctcaagcagctctaccagcgcaccaacccctataagcagttcaaaaatgactccagggtggccttctgctcttttaccaacctcagagagcagtacatccgccggctgattatgaccagcttcatcggctatgtgtttaaggccctgcaggagtggatgccatcctactctaagcccacacacaccacaaaaaccctgctctccgagctgatcaccctcgtggacacactgaagcaggaaacaaacgatgtccctagcgagtccgtggtcaataccatcctgtccattgctgactcttgtaagacccagacacagaagagcaaagaagccaaaaccacaatcgactccttcctgagggagcacttcgtgtttgatcccaacctgcatgctcagagcgcctacacctgcgcttccacaaacgccgacacctctgctagcacaaatgtggacacctgcgtcgatacatgtgccagcatgggagcttccacctgtgccgacacaaatgtggatacctgcgcttctatggatacctgtgccagcaccgaatatacagacctcgccgatcccgagcgcatccccctgcacattatgcagaagaccctgaacgtgcccaatgagctccaggctgacatcgatgccattacccagacacctcaggggtacagagccgctgcccatatcctgcagaacattgaactccaccagagcatcaagcatatgctggagaaccctcgcgccttcaagccaatcctctttaataccaaaattacacggtacctgtcccagcacatcccccctcaggacaccttctacaagtggaactactacatcgaggataactacgaggaactgagggctgccaccgagagcatctatccagaaaagcccgacctggagttcgcctttatcatctacgacgtggtcgatagctccaaccagcagaaggtggacgaattctactacaagtacaaggatcagattttcagcgaggtctctagcattcagctggggaactggaccctgctcggcagcttcaaggccaacagggaaagatacaactacttcaaccagaacaacgagatcatcaagcgcatcctggaccggcacgaggaggacctgaagatcggcaaagaaattctgagaaacaccatctatcataagaaggctaagaacatccaggagaccggacctgacgctccaggactgtctatctacaacagcaccttccacacagattccggcattaaggggctgctctcttttaaggaactgaaaaacctcgagaaggccagcgggaatatcaagaaagcccgcgagtacgacttcatcgacgattgcgaggaaaagatcaagcagctgctctctaaggaaaacctgacaccagacgaggaatccgagctcatcaaaaccaagaaacagctggataacgccctggagatgctcaatgtgcccgacgataccatccgggtcgacatgtgggtgaataacaacaacaaactggagaaagaaatcctctacaccaaagccgaactcaaaaaatacccatacgacgttccggactacgcttcttagtgataa

SEQ ID NO: 20 - protein, 558 aa, African swine fever virus
MGDYKDDDDKKAEFNIDELLKNVLEDPSTEISEETLKQLYQRTNPYKQFKNDSRVAFCSFTNLREQYIRRLIMTSFIGYVFKALQEWMPSYSKPTHTTKTLLSELITLVDTLKQETNDVPSESVVNTILSIADSCKTQTQKSKEAKTTIDSFLREHFVFDPNLHAQSAYTCASTNADTSASTNVDTCVDTCASMGASTCADTNVDTCASMDTCASTEYTDLADPERIPLHIMQKTLNVPNELQADIDAITQTPQGYRAAAHILQNIELHQSIKHMLENPRAFKPILFNTKITRYLSQHIPPQDTFYKWNYYIEDNYEELRAATESIYPEKPDLEFAFIIYDVVDSSNQQKVDEFYYKYKDQIFSEVSSIQLGNWTLLGSFKANRERYNYFNQNNEIIKRILDRHEEDLKIGKEILRNTIYHKKAKNIQETGPDAPGLSIYNSTFHTDSGIKGLLSFKELKNLEKASGNIKKAREYDFIDDCEEKIKQLLSKENLTPDEESELIKTKKQLDNALEMLNVPDDTIRVDMWVNNNNKLEKEILYTKAELKKYPYDVPDYAS

SEQ ID NO: 21 - DNA, 2016 bp, African swine fever virus; identical to SEQ ID NO: 7
  CDS (1)..(2013)

SEQ ID NO: 22 - protein, 671 aa, African swine fever virus; identical to SEQ ID NO: 8

(19) United States
(12) Patent Application Publication
(10) Pub. No.: US 2019/0307879 A1
(43) Pub. Date: Oct. 10, 2019

(54) ADENOVIRUS-VECTORED MULTIVALENT VACCINE

(71) Applicant: THE TEXAS A&M UNIVERSITY SYSTEM, COLLEGE STATION, TX (US)

(72) Inventors: WAITHAKA MWANGI, COLLEGE STATION, TX (US); SURYAKANT D. WAGHELA, COLLEGE STATION, TX (US); SHEHNAZ T. LOKHANDWALA, MANHATTAN, KS (US); JOCELYNE M. BRAY, BRYAN, TX (US)

(21) Appl. No.: 15/781,206

(22) PCT Filed: Dec. 5, 2016
(86) PCT No.: PCT/US2016/064880

§ 371 Date: Jun. 4, 2018

Related U.S. Application Data
(60) Provisional application No. 62/263,424, filed on Dec. 4, 2015.

(51) Int. Cl. A61K 39/21; A61K 9/00; C12N 15/86

(52) U.S. Cl. CPC A61K 39/21; A61K 2039/70; C12N 15/86; A61K 9/0019

(57) ABSTRACT

The invention pertains to a vaccine comprising an immunologically effective amount of a novel live-vectored multivalent vaccine formulation that affords immunization to multiple antigens of a pathogen that is relatively impervious to vaccine development by providing multiple virus-expressed antigens and a pharmaceutically acceptable carrier and/or an adjuvant. Further, a method of immunizing a subject against an exposure to a pathogen that is relatively impervious to vaccine development is provided, wherein the method comprising the steps of administering the vaccine to a subject to induce an immune response against antigenic proteins or fragments thereof.

### CROSS-REFERENCE TO RELATED APPLICATION

This application claims the benefit of U.S. Provisional Application Ser. No. 62/263,424, filed Dec. 4, 2015, the disclosure of which is hereby incorporated by reference in its entirety, including all figures, tables and amino acid or nucleic acid sequences.

This invention was made with government support under HSHQDC-11-C-00116/TAMRF 503671 awarded by Department of Homeland Security (DHS). The government has certain rights in the invention.

The Sequence Listing for this application is labeled “Seq-List.txt” which was created on Dec. 2, 2016 and is 79 KB. The entire contents of the sequence listing is incorporated herein by reference in its entirety.

### BACKGROUND OF THE INVENTION

The African Swine Fever Virus (ASFV) causes a highly contagious fatal hemorrhagic disease in domestic swine and at present, there is no treatment or vaccine available. Currently, isolation and culling are the only methods to control or eradicate ASFV. The USA is the leading pork exporter and it is estimated that an African Swine Fever virus (ASFV) outbreak will cost billions of dollars, jeopardize food security, and compromise foreign trade. This threat poses a real danger to the US swine industry and has been identified as a National food security threat by US National Pork Board and the Department of Homeland Security (DHS). Thus, investing in the development of vaccines capable of containing an ASFV outbreak is critical to safeguard the swine industry and preserve future competitiveness of the US pork industry.

ASFV is highly contagious, easily transmitted, and causes a high-consequence Transboundary Animal Disease (TAD) in pigs with a mortality rate of nearly 100%. Worldwide, the virus has spread much faster in the last five years than it did in the previous fifty years. There is a need to develop counter-measure in preparation for the threat posed by the rapid spread of this pathogen and also for threat reduction in endemic regions to curtail transmission to U.S.A. Development of a vaccine is feasible since pigs that recover from infection with ASFV mutants are protected. However, attenuated ASFV is not a good vaccine and is unlikely to be deployed given that vaccinated pigs become life-long carriers of a mutant virus that is likely to acquire virulent traits.

Previous vaccination studies suggest that induction of ASFV-specific cytotoxic T lymphocytes (CTLs) could be the key to complete protection. Hence, generation of an efficacious subunit ASFV vaccine depends on successful identification of CTL targets and a suitable delivery platform that will prime and expand lytic T-cells capable of eliminating ASFV-infected host cells and confer long-term memory. Current data suggests that subunit vaccines based on a few of the currently defined ASFV antigens are unlikely to induce protective immunity. For example, subunit vaccines based on one or two ASFV antigens have, so far, failed to induce immunity that is strong enough to confer significant protection. It is envisaged that successful development of an effective subunit vaccine will require identification and validation of multiple suitable antigens that will induce significant protection in majority of the vaccinated pigs.

### BRIEF SUMMARY OF THE INVENTION

The invention provides a method of developing vaccines for protection from pathogens that have been impervious to vaccine development (i.e., pathogens against which it is difficult to develop a vaccine). The methods of the instant invention can be applied to the vaccination of mammals including humans and non-human animals (e.g., livestock). In specific embodiments, the instant invention provides novel live-vectored multivalent vaccine formulations against African Swine Fever Virus (ASFV) or an infection-causing microorganism producing ASFV proteins or fragments thereof. In more specific embodiments, the instant invention provides replication-incompetent recombinant adenoviruses for use as a cocktail immunogen, wherein the recombinant adenoviruses encode codon-optimized ASFV antigens that rapidly induce ASFV-specific IgG response, IFN-γ-secreting T cells, and CTL responses.

Advantageously, antibody responses primed with the vaccines of the instant invention undergo rapid isotype-switching within one week and antigen-specific IgG responses increase significantly over a two-month period and undergo rapid recall upon boost four months post-priming. For example, at four months post-priming, titers achieved in experimental animals are as high as 1:8×10⁶.

In other embodiments, a method to immunize subjects is provided, wherein the method comprises the steps of administering the vaccine to a subject to induce an immune response against ASFV proteins in the subject.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1 shows the organization of the generic expression cassette for the lead ASFV antigens.

FIGS. 2A-2G show the amino acid sequences of the ASFV antigens that were used to design synthetic genes codon-optimized for protein expression in swine cells. FIG. 2A: ASFV p32 codon-optimized synthetic gene and amino acid sequences (SEQ ID NOs: 1 and 2). FIG. 2B: ASFV p54 codon-optimized synthetic gene and amino acid sequences (SEQ ID NOs: 3 and 4). FIG. 2C: ASFV p62 codon-optimized synthetic gene and amino acid sequences (SEQ ID NOs: 5 and 6). FIG. 2D: ASFV p72 codon-optimized synthetic gene and amino acid sequences (SEQ ID NOs: 7 and 8). FIG. 2E: ASFV p37 (p37-p43-p14) codon-optimized synthetic gene and amino acid sequences (SEQ ID NOs: 9 and 10). FIG. 2F: ASFV p150-I codon-optimized synthetic gene and amino acid sequences (SEQ ID NOs: 11 and 12). FIG. 2G: ASFV p150-II codon-optimized synthetic gene and amino acid sequences (SEQ ID NOs: 13 and 14).

FIG. 3 shows the workflow for generation of constructs.

FIG. 4 shows the immunocytometric analysis of ASFV antigen expression.

FIGS. 5A-5D show the immunocytometric analysis of Sf9 cells infected with Baculovirus expressing ASFV p72 antigen. FIG. 5A: ASFV-specific serum probe. FIG. 5B: Anti-FLAG mAb probe. FIG. 5C: Anti-HA mAb probe. FIG. 5D: Non-infected cells.

FIG. 6 shows protein expression by pLenti DNA constructs using an anti-V5 mAb probe.

FIG. 7 shows a western blot analysis of affinity purified proteins.

FIGS. 8A-8B show a flow cytometric analysis of GFP expression in HEK293A cells infected with recombinant Lentivirus constructs expressing p37 and p72. FIG. 8A: Small scale lentivirus preparation. FIG. 8B: scaled up lentivirus preparation.

FIGS. 9A-9B show IFA evaluation of rabbit anti-p62 polyclonal antibodies. FIG. 9A shows titration of serum from rabbit #DAG31 on ASFV Georgia-Infected swine macrophages. FIG. 9B shows titration of serum from rabbit #DAG32 on ASFV Georgia-Infected swine macrophages.

FIGS. 10A-10B show IFA evaluation of rabbit anti-p54 polyclonal antibodies.

FIG. 10A shows titration of serum from rabbit #DAG33 serum on ASFV Georgia-infected swine macrophages. FIG. 10B shows titration of serum from rabbit #DAG33 serum on ASFV BA71V-infected VERO cells.

FIG. 11 shows IFA evaluation of mouse anti-p54 and mouse anti-p62 sera.

FIG. 12 shows the summary of the in vivo study time line.

FIGS. 13A-13D show an Ad5-ASFV cocktail rapidly primed antibody response. FIG. 13A: Anti-p32 IgM and IgG responses at 1 week post-prime. FIG. 13B: Anti-p54 IgM and IgG responses at 1 week post-prime. FIG. 13C: Anti-p62 IgM and IgG responses at 1 week post-prime. FIG. 13D: Anti-p37 IgM and IgG responses at 1 week post-prime.

FIGS. 14A-14D show antigen-specific IgG profiles post-priming. FIG. 14A: Anti-p32 IgG responses at 1, 2, 4, and 6 weeks post-prime. FIG. 14B: Anti-p54 IgG responses at 1, 2, 4, and 6 weeks post-prime. FIG. 14C: Anti-p62 IgG responses at 1, 2, 4, and 6 weeks post-prime. FIG. 14D: Anti-p37 IgG responses at 1, 2, 4, and 6 weeks post-prime.

FIGS. 15A-15D show antigen-specific IgG profiles 8-10 weeks post-priming. FIG. 15A: Anti-p32 IgG responses at 8 and 10 weeks post-prime. FIG. 15B: Anti-p54 IgG responses at 8 and 10 weeks post-prime. FIG. 15C: Anti-p62 IgG responses at 8 and 10 weeks post-prime. FIG. 15D: Anti-p37 IgG responses at 8 and 10 weeks post-prime.

FIGS. 16A-16D show recall antigen-specific IgG profiles post-boost. FIG. 16A: Anti-p32 IgG responses at 1 week post-boost. FIG. 16B: Anti-p54 IgG responses at 1 week post-boost. FIG. 16C: Anti-p62 IgG responses at 1 week post-boost. FIG. 16D: Anti-p37 IgG responses at 1 week post-boost.

FIG. 17 shows Indirect Immunofluorescence Antibody Assay (IFA) results using primary swine macrophages infected with the ASFV George 2007/1 isolate.

FIGS. 18A-18B show western blot results using lysates from Vero cells infected with the ASFV George 2007/1 isolate. FIG. 18A: Lane 1: superpig serum; Lane 2: Mwt marker; Lane 3: normal swine serum; Group 1: sera 4-8; Group 2: sera 9-13; Group 3: sera 14-18; Group 4 sera 19-23. Sera 4-23 correspond to the pig number shown in Table 3. FIG. 18B: Sera numbers 1-20 correspond to the pig numbers shown in Table 3.

FIGS. 19A-19B show EliSpot results 2 weeks post-prime of Ad5-ASFV cocktail primed IFN-γ-secreting cells. FIG. 19A: p54-specific IFN-γ-specific EliSpot. FIG. 19B: p62-specific IFN-γ Eli Spot.

FIGS. 20A-20E show antigen-specific IFN-γ responses 8 weeks post-priming. FIG. 20: p32-specific IFN-γ-specific IFN-γ EliSpot. FIG. 20B: p54-specific IFN-γ EliSpot. FIG. 20C: p62-specific IFN-γ EliSpot. FIG. 20D: p37-specific IFN-γ EliSpot. FIG. 20E: p150-I-specific IFN-γ EliSpot.

FIGS. 21A-21D show antigen-specific IFN-γ recall responses 1 week post-boost. FIG. 21A: p54-specific IFN-γ-specific EliSpot. FIG. 21B: p62-specific IFN-γ EliSpot. FIG. 21C: p37-specific IFN-γ EliSpot. FIG. 21D: p150-I-specific IFN-γ EliSpot.

FIGS. 22A-22G show antigen-specific IFN-γ recall responses in splenocytes 1 week post-boost. FIG. 22A: p32-specific IFN-γ-specific EliSpot. FIG. 22B: p54-specific IFN-γ EliSpot. FIG. 22C: p62-specific IFN-γ EliSpot. FIG. 22D: p72-specific IFN-γ EliSpot. FIG. 22E: p37-specific IFN-γ EliSpot. FIG. 22F: p150-I-specific IFN-γ EliSpot. FIG. 22G: p150-II-specific IFN-γ EliSpot.

FIG. 23 shows IFN-γ responses to predicted SLA-I binding peptides.

FIGS. 24A-24H shows antigen-specific CTL responses. FIG. 24A: One round of in vitro stimulation pig #33. FIG. 24B: One round of in vitro stimulation pig #35. FIG. 24C: One round of in vitro stimulation pig #36. FIG. 24D: One round of in vitro stimulation pig #40. FIG. 24E: One round of in vitro stimulation pig #42. FIG. 24F: One round of in vitro stimulation pig #46. FIG. 24G: One and two rounds of in vitro stimulation pig #37. FIG. 24H: One round of in vitro stimulation pig #93.

FIG. 25 shows the summary of in vivo study time line for Ad5-ASFV 4-way cocktail vaccinations.

FIG. 26 shows the immunocytometric analysis of HEK293A cells infected with adenoviruses expressing the A151R, B119L, B602L, and B646L antigens, respectively.

FIG. 27A shows the immunocytometric analysis of Sf9 cells infected with baculoviruses expressing the A151R (A), B119L (B), B602L (C), and B646L (D) antigens, respectively. FIG. 27B shows a western blot analysis of the affinity purified ASFV proteins probed with ASFV superpig serum (1:5000 dilution): secondary antibody: Anti-porcine AP 1:1000, substrate: Immunostar AP; lane 1: Mwt marker in kDa; lane 2: A151R; lane 3: B119L; lane 4: B602L; and lane 5: B646L.

FIGS. 28A-28B show ASFV antigen-specific (FIG. 28A) anti-A151R and anti-B646L and (FIG. 28B) antiB602L and anti-B646L IgG responses post priming.

FIGS. 29A-29B show recall ASFV antigen-specific (FIG. 29A) anti-A151R and anti-B119L and (FIG. 29B) antiB602L and anti-B646L IgG responses post-boost.

FIGS. 30A-30B show ASFV antigen-specific (FIG. 30A) anti-A151R and anti-B119L and (FIG. 30B) antiB602L and anti-B646L IgG endpoint titers post-boost.

FIG. 31 shows western blots of primed antibodies recognizing native ASFV antigens.

FIG. 32 shows representative IFA images of Vero cells infected with ASFV George 2007/1 and probed with antibodies obtained from adeno-ASF cocktail-vaccinated pigs.

FIGS. 33A-33D show post-priming EliSpot data of IFN-γ-secreting cells of pigs vaccinated with chaperone-substrate pairs of (FIG. 33A) A151R- and B119L-, and (FIG. 33B) B602L- and B646L-expressing adenoviruses. FIG. 33C and FIG. 33D show post-boost EliSpot data of IFN-γ-secreting cells of pigs vaccinated with chaperone-substrate pairs of (FIG. 33C) A151R- and B119L-, and (FIG. 33D) B602L- and B646L-expressing adenoviruses.

Table 1 shows the list of constructs generated.

Table 2 shows monoclonal antibody reactivity on ASFV- and mock-infected VERO cells.

Table 3 shows hybridomas selected for subcloning.

Table 4 shows deliverable rabbit polyclonal and mouse monoclonal antibodies.

Table 5 shows the immunization protocol.

Table 6 shows the summary of immunogen safety and tolerability.

Table 7 shows that IFA reactivity of swine anti-ASFV sera.

Table 8 shows the immunization protocol of Ad5-ASFv 4-way cocktail (AdA151R, AdB119L, AdB602L, and AdB646L) vaccinated pigs.

Table 9 shows the reactivity of primed antibodies recognizing ASF virus.

Table 10 shows the summary of vaccine safety and tolerability data in Ad5-ASFV 4-way cocktail vaccinated pigs.

### BRIEF DESCRIPTION OF THE SEQUENCES

SEQ ID NOs: 1-2: Codon-optimized synthetic nucleotide sequence of ASFV p32 wherein ASFV p17 and p12 were fused in-frame to p32 to generate a chimera.

SEQ ID NOs: 3-4: Codon-optimized synthetic nucleotide sequence of ASFV p54.

SEQ ID NOs: 5-6: Codon-optimized synthetic nucleotide sequence of ASFV p62.

SEQ ID NOs: 7-8: Codon-optimized synthetic nucleotide sequence of ASFV p72.

SEQ ID NOs: 9-10: Codon-optimized synthetic nucleotide sequence of ASFV p37, wherein ASFV p34 and p14 were fused in-frame to p37 to generate a chimera.

SEQ ID NOs: 11-12: Codon-optimized synthetic nucleotide sequence of ASFV p150-I.

SEQ ID NOs: 13-14: Codon-optimized synthetic nucleotide sequence of ASFV p150-II.

SEQ ID NOs: 15-16: A151R.

SEQ ID NOs: 17-18: B119L.

SEQ ID NOs: 19-20: B602L.

SEQ ID NOs: 21-22: B646L.

### DETAILED DISCLOSURE OF THE INVENTION

The instant invention provides multi-component vaccines, methods for generating said vaccines, and methods of immunization subjects to protect against pathogens that have been impervious to vaccine development. In specific embodiments, the instant invention provides vaccines based on replication-incompetent recombinant virus-generated multivalent antigen cocktails for safe immunization and rapid induction of pathogen-specific humoral and cellular immune responses. In certain embodiments, the multi-valent antigen cocktail is generated using replication-incompetent recombinant lentivirus-based vector systems carrying pathogen-specific antigens. In other embodiments, the multi-valent antigen cocktail is generated using replication-incompetent recombinant adenovirus-based expression systems carrying pathogen-specific antigenic proteins or fragments thereof. In preferred embodiments, the multi-valent antigen cocktail is generated using a replication-incompetent recombinant adenovirus type 5 (Ad5)-based vector system carrying pathogen-specific antigenic proteins or fragments thereof. In more preferred embodiments, the multi-valent antigen cocktail is generated using a replication-incompetent recombinant adenovirus type 5 (Ad5)-based vector system carrying antigens specific for African Swine Fever Virus (ASFV).

In various embodiments, the multi-valent replication-incompetent recombinant viruses encode codon-optimized antigenic proteins or fragments thereof. For codon optimization, the nucleic acids encoding the antigenic proteins or fragments thereof are modified to allow codon usage that is preferred in the vaccinee species. For example, antigenic proteins or fragments thereof of ASFV to be expressed in a swine are codon-optimized with reference to codon usage in the swine. The skilled artisan is familiar with the technique of codon optimization and can adapt the technique to different species.

In some embodiments, antigenic protein sequences are aligned with common pathogenic virus reference sequences and consensus sequences, if present, are chosen to enable the generation of antigenic proteins or fragments thereof that offer immunity to a broad array of pathogens. In other embodiments, where no consensus sequence(s) with one or more reference sequence(s) exist, either the pathogen-specific sequences or the consensus sequence can be chosen to generate multi-valent antigenic cocktails according to the methods of the instant invention. In preferred embodiments, the multi-valent replication-incompetent recombinant Ad5-viruses encode codon-optimized ASFV antigens that are efficiently expressed in cells of pigs.

Advantageously, the use of live virus-based vectors to deliver multiple defined pathogen-specific antigens increases antigen immunogenicity and leads to the induction of efficient antibody responses, significant IFN-γ responses and very strong pathogen-specific Cytotoxic T Lymphocyte (CTL) responses.

In many embodiments, the instant invention provides replication-incompetent recombinant viruses for use as cocktail immunogens, wherein the several recombinant viruses encode multiple codon-optimized pathogen-specific antigenic proteins or fragments thereof that efficiently express multiple pathogenic antigens in target subjects and rapidly induce pathogen-specific IgG responses, IFN-γ-secreting T cells, and CTL responses.

The term “subject” refers to an animal, such as a human or non-human mammal. Non-limiting examples of non-human mammals in which the methods of the invention can be practiced include dogs, cats, pigs, cattle, rabbits, sheep, goats, deer, horses, rodents, apes, chimpanzees, orangutans and monkeys. Additional examples of subjects in which the methods of the invention can be practiced are well known to a person of ordinary skill in the art and such embodiments are within the purview of the invention. Where a subject is vaccinated, the subject may be referred to as a “vaccinee”.

The phrase “virus-based vector system” refers to protein expression constructs based on baculoviruses, adenoviruses and lentiviruses.

“Codon-optimized” genes refer to genetic sequences of target pathogens that are modified to include codons that are preferentially expressed in the vaccinee to be treated using the virus-based vaccine of the instant invention.

In some embodiments, antigenic proteins or fragments thereof are modified to add, in-frame, tags, including, for example, FLAG- and HA-tags at the N- and C-terminus to aid tracking protein expression and affinity purification. In other embodiments, two tags are included to allow the use of one primer pair to move the expression cassettes across multiple expression vectors. In most embodiments, codon-optimized antigen-encoding sequences are incorporated into viral vector genomes of viruses used in the multi-valent antigen cocktails to enable expression of multiple antigenic proteins or fragments thereof by the respective live viruses following vaccination.

In preferred embodiments, antigenic proteins or fragments thereof are selected from, for example, ASFV proteins, including, but not limited to, p32, -54, p62, p72 and p220 of ASFV. In further preferred embodiments, the antigens are selected from any of SEQ ID NOs: 1-14. In some embodiments, the p220 antigen is divided into peptides p37, p150-I and p150-II, wherein p37 comprises p37 conjugated to p34 and p14, respectively. In other preferred embodiments, the sequences of antigenic proteins or fragments thereof are fused in-frame to generate chimeric antigenic proteins or fragments thereof. For example, the nucleic acid sequence of p32 of ASFV can be fused to ASFV p17 and p12 to generate an antigenic chimera (SEQ ID NOs: 1-2). Alternatively, the nucleic acid sequence of p37 of ASFV can be fused to ASFV p34 and p14 to generate an antigenic chimera (SEQ ID NOs: 9-10).

In other preferred embodiments, the antigenic protein or fragment thereof is derived from B119L or B646L of ASFV. The B119L antigen is an attractive candidate because it is critical for virus assembly, B119L is immunogenic and is highly conserved amongst all ASFV isolates studied to-date. B646L is a major capsid protein of ASFV and immunization with the B646L antigen induces antibodies capable of inhibiting binding of the ASF virus to permissive cells. In addition, B646L has been shown to induce lymphocytes that are capable of killing swine cells infected with ASFV. However, B119L and B646L are generally poorly expressed by live vectors. Advantageously, A151R is a natural chaperone for B119L and B602L is a natural chaperone for B646L.

In certain embodiments, the vaccine of the instant invention provides cocktails of recombinant viruses expressing ASFV proteins A151R, B119L, B602L and B646L (e.g., separately by individual viruses of the multi-valent antigen cocktails). The cocktail can be any combination of two, three or four individual viruses expressing ASFV proteins A151R, B119L, B602L and B646L. In other embodiments, B119L and A151R are expressed in a single recombinant virus and B646L and B602L are expressed in another recombinant virus. Yet other embodiments provide a recombinant virus expressing B119L and one or two ASFV proteins selected from the group consisting of A151R, B646L and B602L. Other embodiments provide for the a recombinant virus expressing the following combinations of ASFV antigens (A=B119L; B=A151R; C=B646L and D=B602L): A and B; A and C; A and D; B and C; B and D; C and D; A and B and C; A and B and D; A and C and D; and B and C and D. Certain preferred embodiments provide recombinant virus cocktails that express the following combination of ASFV proteins in a subject: A151R, B119L, B602L and B646L. It has been found that the co-expression of chaperones A151L and B602L with B119L and B646L aids in the stability and expression of B119L and B646L ASFV antigenic proteins and leads to ASFV-specific IFN-γ-secreting cells and strong ASFV antigen-specific IgG responses, both of which undergo rapid recall upon boost with the priming recombinant virus cocktail. As discussed above, in certain embodiments, nucleic acid sequences encoding the ASFV proteins A151R, B119L, B602L, and B646L are codon-optimized to yield high expression in the respective vaccinee.

In many embodiments of the instant invention, multi-valent antigen cocktails comprise adenoviruses expressing antigenic proteins or fragments thereof. In other embodiments, multi-valent antigen cocktails comprise lentiviruses expressing antigenic proteins or fragments thereof. In yet other embodiments, multi-valent antigen cocktails comprise baculoviruses expressing antigenic proteins or fragments thereof, wherein the baculoviruses are modified with mammalian promoters to enable protein expression in mammalian cells.

In most embodiments, the recombinant viruses constituting the multi-valent antigen cocktail are administered simultaneously. Alternatively, one or a group of first recombinant viruses can be administered before or after one or a group of second recombinant viruses. The terms “simultaneous” or “simultaneously” as applied to administering vaccines to a subject refer to administering one or more vaccines at the same time, or at two different time points that are separated by no more than 30 minutes. The term “after or before” as applied to administering vaccines to a subject refers to administering more than one doses at two different time points that are separated by more than 30 minutes, e.g., about 1 hour, about 2 hours, about 5 hours, 8 hours, 1 day, 2 days, 3 days, 4 days, 5 days, 6 days, 7 days or even longer.

In preferred embodiments, the multi-valent antigen cocktail of the instant invention is administered at a first time point to induce an antigen-specific immune response, i.e. prime the vaccinee, and again administered at a second time point to boost said antigen-specific immune response. Advantageously, the multi-valent antigenic vaccine based on a cocktail of recombinant adenoviruses expressing ASFV-derived antigenic proteins or fragments thereof efficiently induces antigen-specific CTLs and IFN-γ-secreting T cells and splenocytes in vaccinees.

In some embodiments, polyclonal and monoclonal antibodies are created using the multi-valent antigen cocktails of the instant invention. For example, rabbits or mice are vaccinated with the multi-valent antigen cocktail that has comprise recombinant viruses that encode codon-optimized target antigens for efficient expression in rabbit or mouse respectively, and in vivo generated antibodies are retrieved using routine techniques well-known to the skilled artisan. Advantageously, polyclonal and monoclonal antibodies can be used for passive immunization of subjects that may not be amenable to active immunization, i.e. immunosuppressed subjects, or to achieve immediate protection in acutely-infected subjects.

In some embodiments, the instant invention provides polynucleotides encoding antigenic proteins or fragments thereof to generate recombinant viruses expressing said antigenic proteins or fragments thereof, which viruses can be included either alone or in combination with other similarly generated recombinant viruses in the multi-valent antigenic cocktail vaccine of the instant invention. In many embodiments, the multi-valent antigenic cocktail vaccine also comprises a pharmaceutically acceptable carrier and/or an adjuvant.

In some embodiments, the antigenic proteins or fragments thereof are present within the recombinant viral nucleic acid as fusion constructs to allow expression of the antigenic proteins or fragment thereof as fusion proteins. For example, fusion proteins can be designed to target Fc receptors, C-type lectins, complement receptors, major histocompatibility proteins, or other receptors present on the surface of dendritic cells or antigen presenting cells. Additional examples of suitable target biomolecules and corresponding binding biomolecules are well known to a person of ordinary skill in the art and such embodiments are within the purview of the invention.

In certain embodiments, the antigenic proteins or fragment thereof are conjugated to heterologous proteins, such as carrier proteins. Non-limiting examples of carrier proteins include dendritic cell targeting peptide (DC-pep), ovalbumin, or bovine serum albumin.

In specific embodiments, the multi-valent antigenic cocktail vaccine is comprised of antigenic proteins or fragments thereof having the sequences of SEQ ID NOs: 1 to 18, and pharmaceutically acceptable carrier and/or an adjuvant.

Antigenic proteins from a pathogenic organism can be identified based on sequence homology and/or activity and such antigenic proteins or fragments thereof can be used in a vaccine to immunize against infection caused by such pathogenic organism. A person of ordinary skill in the art can identify antigenic proteins or fragments thereof in additional pathogenic organisms producing antigenic proteins or fragments thereof and such embodiments are within the purview of the invention.

The fragment of antigenic proteins used in the multi-valent antigenic cocktails of the vaccines of the instant invention can comprise about 5 to about 50, about 10 to about 40, about 15 to about 30, about 20, about 10 or about 5 amino acids. In preferred embodiments, the antigenic proteins or fragments thereof are selected from SEQ ID NOs: 1 to 18.

### Additional Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of ingredients where the terms “about” or “approximately” are used, these compositions contain the stated amount of the ingredient with a variation (error range) of 0-10% around the value (X±10%).

The terms “polypeptide,” “peptide” and “protein” are used interchangeably herein to refer to amino acid chains of any length, including full length proteins recited herein. The terms apply to amino acid polymers in which one or more amino acid residue is an analog or mimetic of a corresponding naturally occurring amino acid, as well as to naturally occurring amino acid polymers. Where the terms “about” or “approximately” are used in the context of peptide sizes, e.g., “about five amino acids”, the terms represent a 10% variation in size. Where the variation in size results in a fraction of an amino acid for the peptide size, the peptide size can be rounded up or rounded down. For example, a peptide size of “about 5 amino acids” represents a peptide that is between 4 and 6 amino acids in length. Similarly, a peptide that is 12 amino acids in length represents a peptide that is 12±1.2 amino acids (10.8 to 13.2 amino acids in length). Where the term “about” is used for such a peptide, the peptide can be between 10 and 14 acids in length.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. Thus, when ranges are used herein, such as for dose ranges, ranges of amino acids, etc., combinations and subcombinations of ranges (e.g., subranges within the disclosed ranges), are intended to be explicitly included.

The term “antibody” refers to a polypeptide substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies applicable according to the present invention can be in various forms, including a whole immunoglobulin, an antibody fragment such as Fab, Fab′, F(ab′)₂, Fv region containing fragments, and similar fragments, as well as a single chain antibody that includes the variable domain complementarity determining regions (CDR), and similar forms. Antibodies within the scope of the invention can be of any isotype, including IgG, IgA, IgE, IgD, and IgM. IgG isotype antibodies can be further subdivided into IgG1, IgG2, IgG3, and IgG4 subtypes. IgA antibodies can be further subdivided into IgA1 and IgA2 subtypes.

“Specific binding” or “specificity” refers to the ability of an antibody or other agent to exclusively bind to an epitope presented on an antigen or peptide while having relatively little non-specific affinity with other proteins or peptides. Specificity can be relatively determined by binding or competitive binding assays, using, e.g., Biacore instruments. Specificity can be mathematically calculated by, e.g., an about 10:1, about 20:1, about 50:1, about 100:1, 10,000:1 or greater ratio of affinity/avidity in binding to the specific antigen or peptide versus nonspecific binding to other irrelevant molecules.

“Immunoassay” is an assay that uses an antibody to specifically bind an antigen or peptide. The immunoassay is characterized by the use of specific binding properties of a particular antibody to a particular antigen or peptide to isolate, target, and/or quantify the antibody. Under designated immunoassay conditions, the specified antibodies bind to a particular protein or peptide at least two times the background and do not substantially bind in a significant amount to other proteins or peptides present in the sample. Specific binding to an antibody under such conditions may require an antibody that is selected for its specificity for a particular protein or peptide. Typically a specific or selective reaction will be at least twice background signal or noise and more typically more than 10 to 100 times background.

“Immunofluorescence Antibody Assay” (IFA) is an assay that uses an antibody to specifically bind an antigen or peptide, wherein the antibody is conjugated to a fluorescent dye to allow detection of antibody binding to antigen using fluorescence microscopy and/or Fluorescence Activated Cell Sorting (FACS).

For the purposes of this invention the term “immunologically effective amount” of an antigenic protein or fragment thereof refers to the amount of the antigenic protein or fragment thereof which, when administered to a subject, elicits adequate immune response in the subject to protect the subject from future infection caused by a microorganism producing the antigenic protein or fragment thereof or exposure to the antigenic protein or fragment thereof.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the antigen in the vaccine, its use in the vaccine compositions of the invention is contemplated.

The vaccine of the invention can be formulated using adjuvants, emulsifiers, pharmaceutically-acceptable carriers or other ingredients routinely provided in a vaccine. Optimum formulations can be readily designed by one of ordinary skill in the art and can include formulations for immediate release and/or for sustained release, and for induction of systemic immunity (e.g., the formulation can be designed for oral, subcutaneous, intraperitoneal, intravenous, intramuscular administration) and/or induction of localized mucosal immunity (e.g., the formulation can be designed for intranasal, intravaginal or intrarectal administration).

Guidelines for designing optimal vaccines can be found in Brito et al. The contents of Brito et al. are herein incorporated by reference in their entirety, particularly, page 132, Table 1; page 133 under immune potentiator adjuvants; page 133-136 under aluminum salt adjuvants; page 136-139 under emulsions; 139-140 under liposomes as adjuvants; page 140-141 under PLG particulate delivery systems; and page 141 under alternate particulate systems. The vaccine disclosed herein can be formed with a pharmaceutically acceptable carrier such as a phosphate buffered saline, a bicarbonate solution, or an adjuvant to produce a pharmaceutical composition. The carrier must be “acceptable” in the sense that it is compatible with the active ingredient of the composition, and preferably capable of stabilizing the active ingredient and not deleterious to the subject to be treated. The carrier is selected on the basis of the mode and route of administration and standard pharmaceutical practice. Suitable pharmaceutical carriers and diluents, as well as pharmaceutical necessities for their use, are described in Remington's Pharmaceutical Sciences.

In one embodiment, the virus expressing the antigen is mixed with an adjuvant to form a composition useful for immune modulation. This composition may be prepared as injectable, as liquid solutions or as emulsions. See U.S. Pat. Nos. 4,601,903; 4,599,231; 4,599,230; and 4,596,792. An “adjuvant” refers to a substance added to an immunogenic composition, such as a vaccine, that, while not having any specific antigenic effect in itself, can stimulate the immune system and increase the immune response to the immunogenic composition. Examples of adjuvants include, but are not limited to, alum, alum-precipitate, Freund's complete adjuvant, Freund's incomplete adjuvant, monophosphoryl-lipid A/trehalose dicorynomycolate adjuvant and water in oil emulsions. Alternatively, multi-valent viral cocktails comprising adenoviruses as disclosed herein can also be used without an adjuvant as the adenoviruses are immunogenic.

The method of the invention can be used to immunize a subject, for example, a mammal, against an infection by a pathogen or an exposure to antigenic proteins or fragments thereof. The vaccine of the invention can be administered by any convenient route including subcutaneous, intradermal, intranasal, oral, intramuscular, intraperitoneal, or other parenteral or enteral route. A person of ordinary skill in the art can identify a particular route of administration suitable for a particular subject and a given antigenic cocktail and such embodiments are within the purview of the invention.

Multi-valent antigen cocktails of the instant invention can be administered as a single dose or multiple doses. Optimum immunization schedules can be determined by the ordinarily skilled artisan and can vary with parameters, for example, age, weight and species of the subject, the type of vaccine composition and the bacterium against which immunization is desired and such embodiments are within the purview of the invention.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLES

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### Example 1—Generation of Constructs Encoding Lead Vaccine Candidate Antigens

Protein expression constructs (Baculovirus, mammalian, adenovirus, and Lentivirus) were generated encoding candidate synthetic genes (p32, p54, pp62, p′72, and pp220 polyprotein [it was split into p3′7; p150-I and p150-II due to its large size]) and modified to contain HA- and FLAG-tags fused in-frame at the 5′ and 3′ ends, respectively.

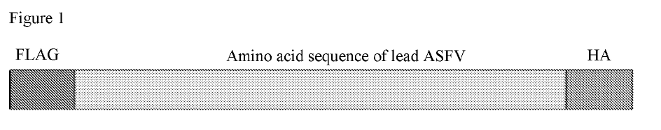

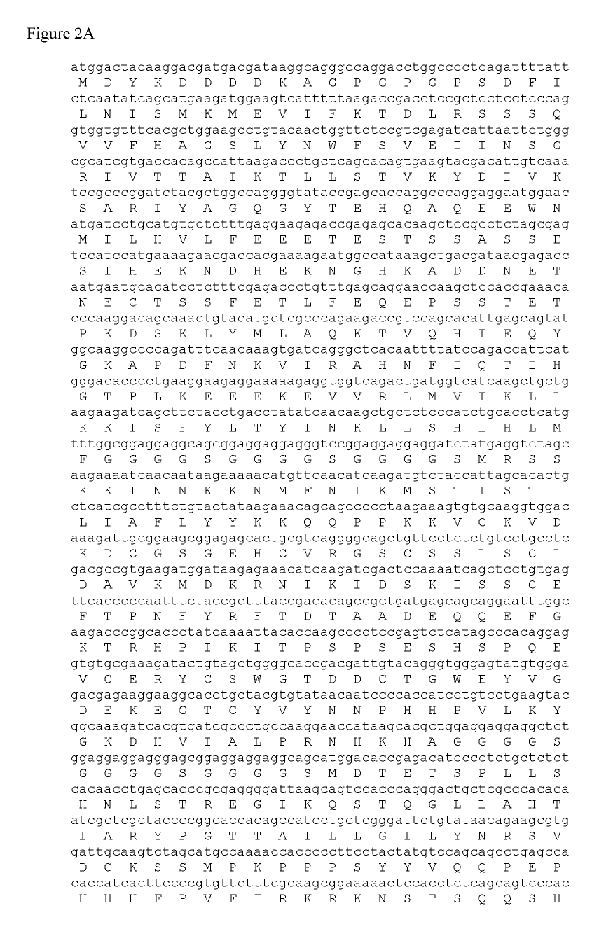

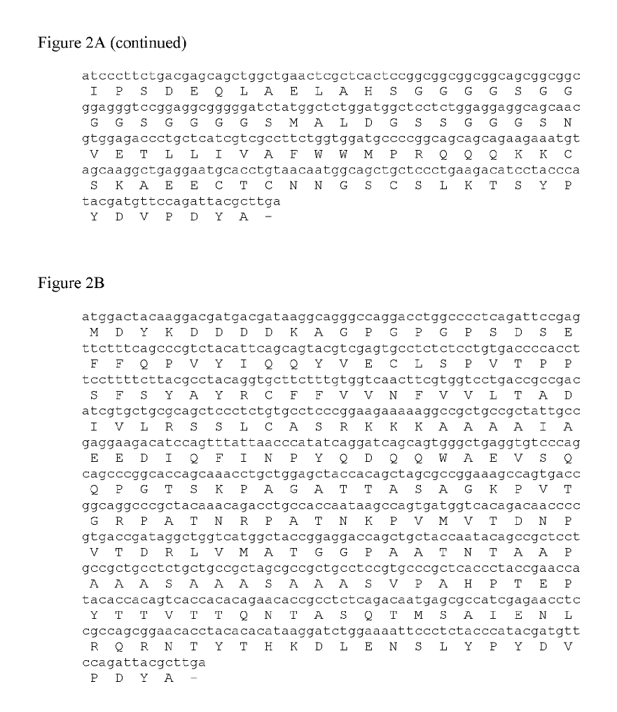

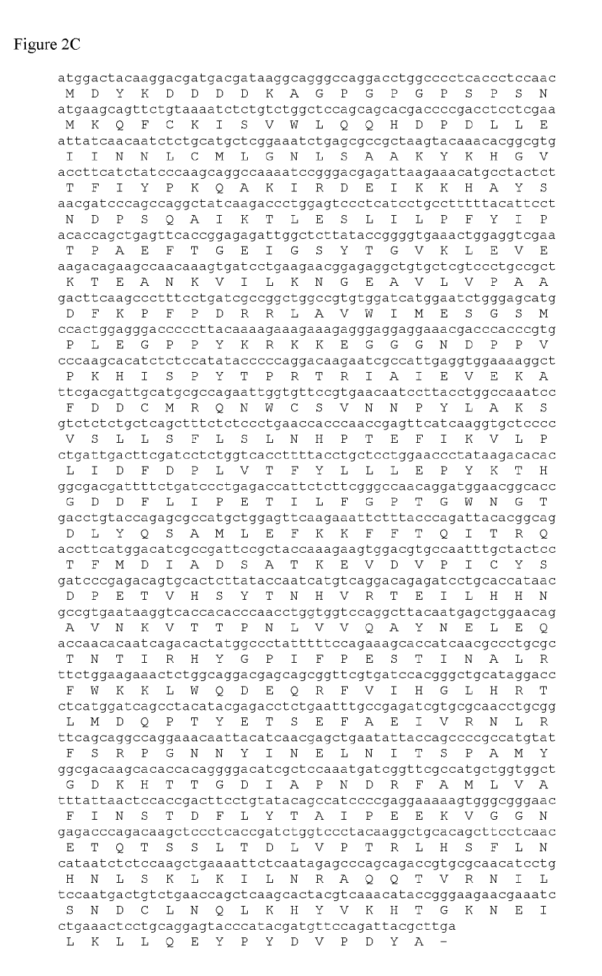

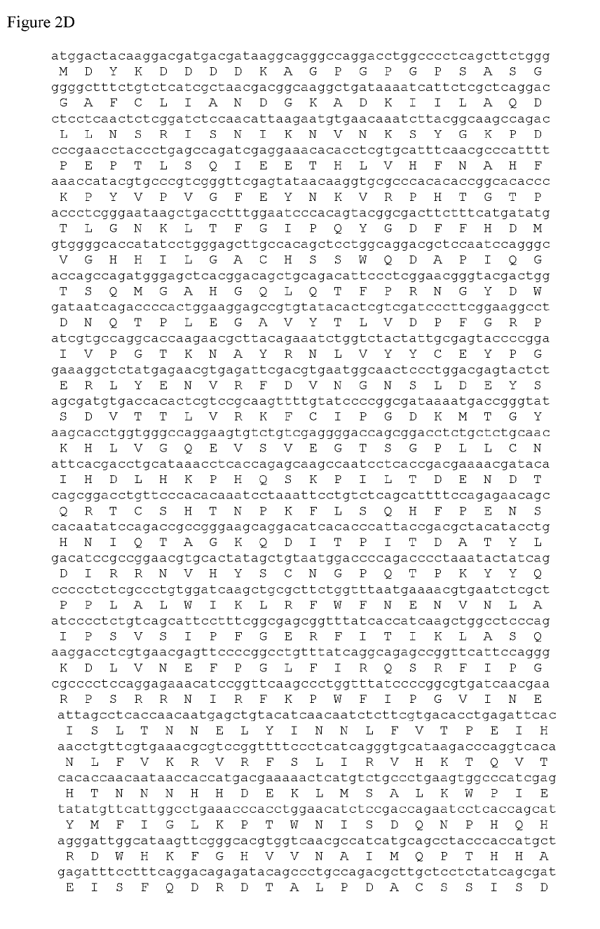

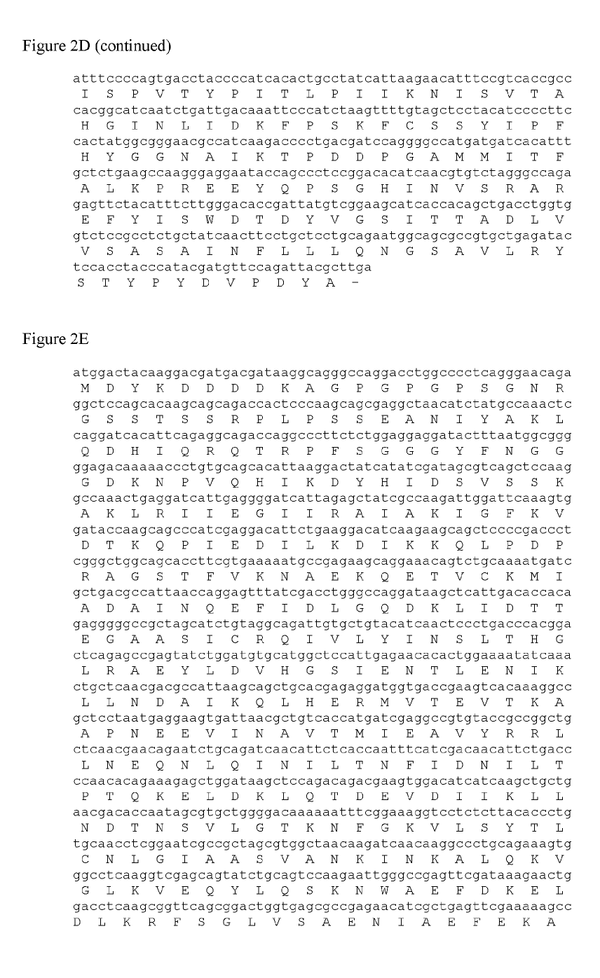

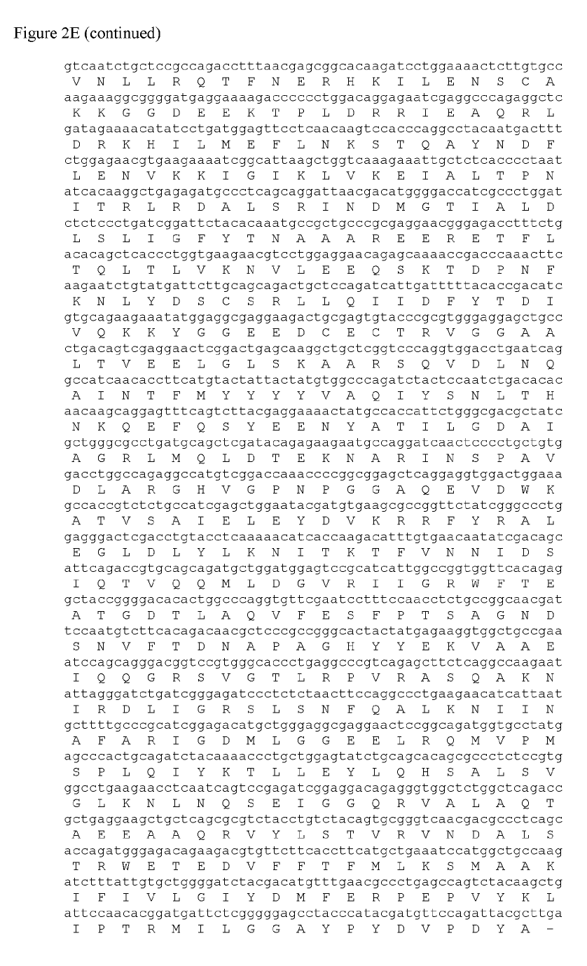

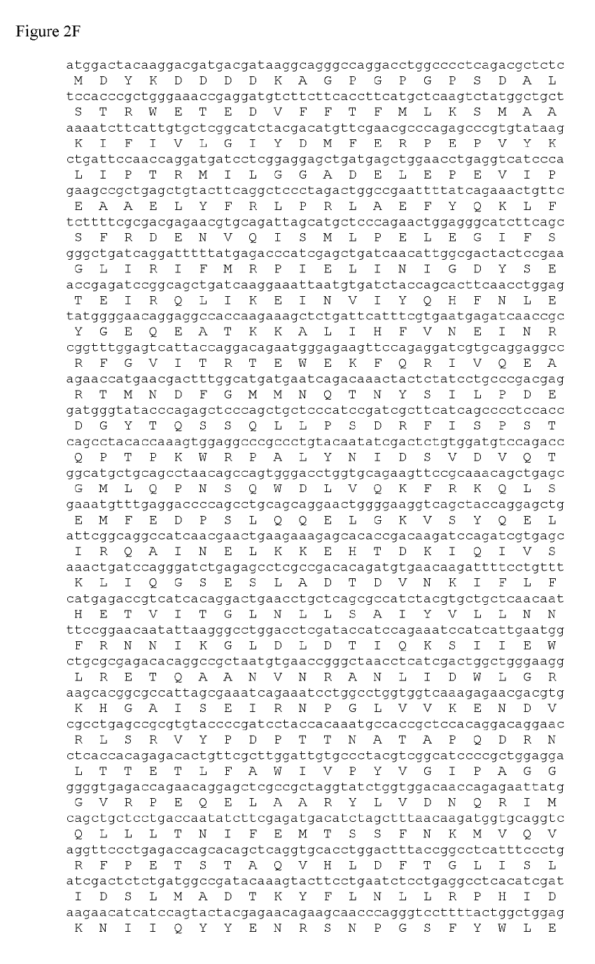

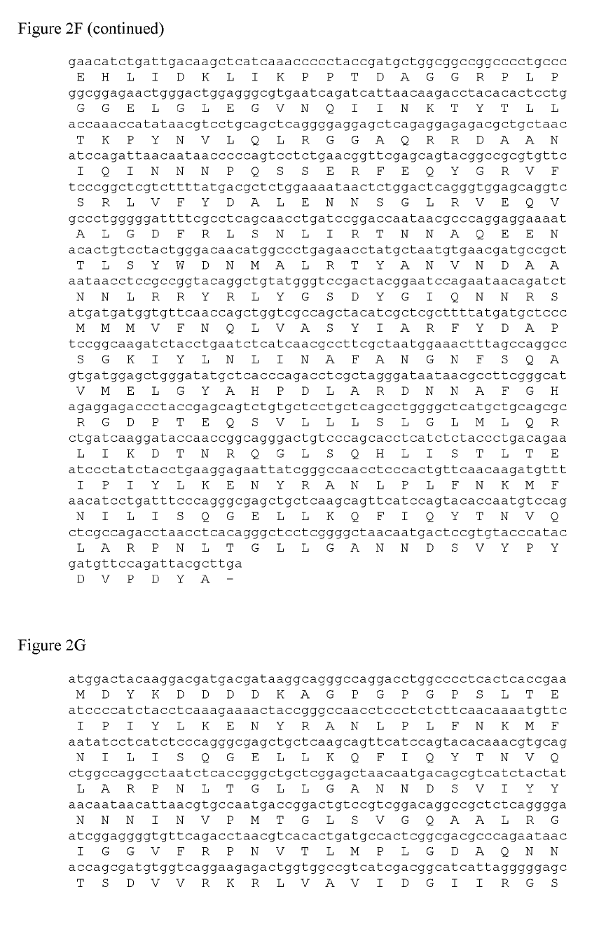

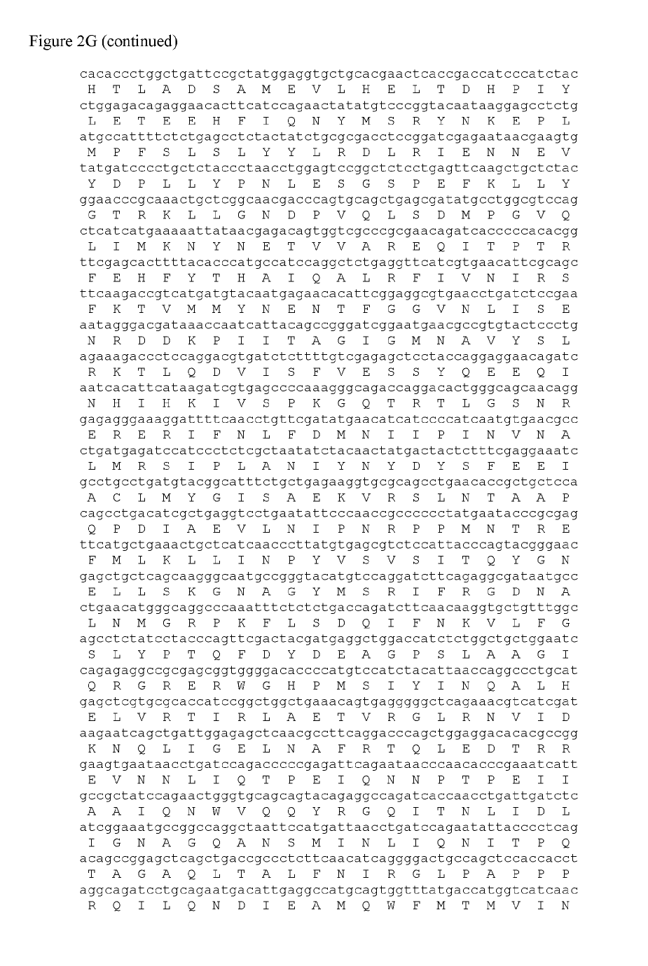

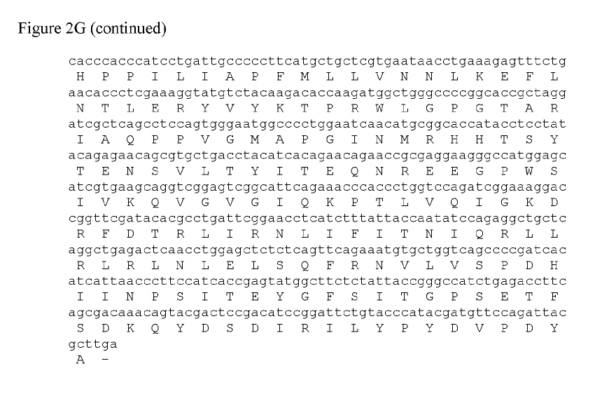

1. Generation of codon-optimized genes and design of expression cassette: The ASFV p32, p54, pp62 polyprotein (p62), p72, and pp220 (p37 [p37-p34-p14]; p150-I; and p150-II) amino acid sequences from all the currently sequenced genomes were aligned and using the George 2007/1 as the reference sequence, consensus amino acid sequences were identified and selected for this study. In most cases where there was no consensus sequence, Georgia 2007/1 amino acid sequences were selected. The amino acid sequence of each antigen was modified to add, in-frame, a FLAG- and HA-tag at the N- and C-termini, respectively to generate an expression cassette as shown in FIG. 1. The inclusion of two tags was a strategy to allow use of one primer pair to move the expression cassettes across multiple expression vectors using the Getaway technology (Invitrogen) in addition to using the tags for tracking protein expression and affinity purification of recombinant protein. The resultant amino acid sequences (FIGS. 2A-2G) of the ASFV antigens were used to design synthetic genes codon-optimized for protein expression in swine cells (FIGS. 2A-2G). Codon optimization and gene synthesis was outsourced from GenScript.

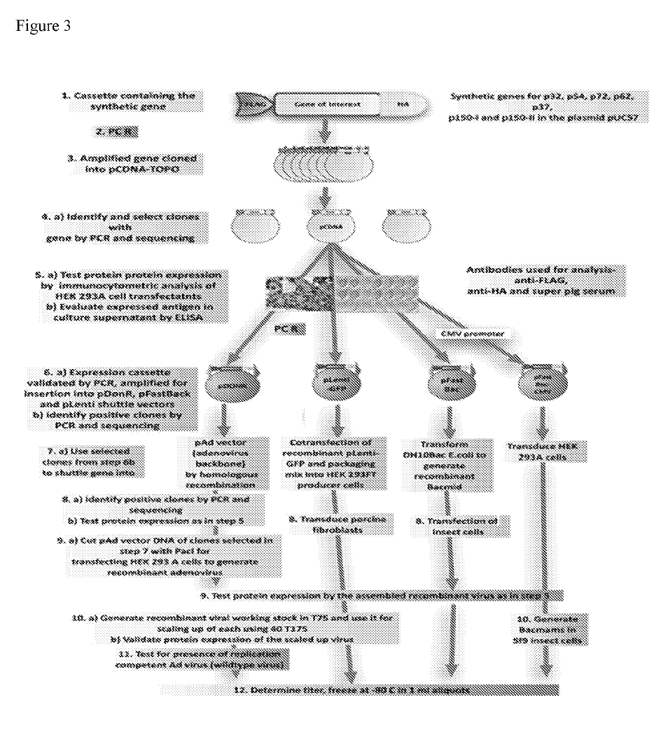

2. Generation of mammalian expression plasmid DNA constructs: The synthetic genes encoding ASFV p32, p54, p72, p62, p37, p150-I, and p150-II were first cloned into the pCDNA3-TOPO mammalian expression vector (Invitrogen) and positive clones were identified by PCR screening and DNA sequencing. Gene cloning/screening/sequence validation workflow is summarized in FIG. 3. Three clones of each construct were selected, miniprep DNA was generated, and aliquots of stock DNA were frozen at −80° C. Seven sequence-verified pCDNA3 constructs encoding the ASFV p32, p54, p72, p62, p37, p150-I, and p150-II antigens were obtained (Table 1).

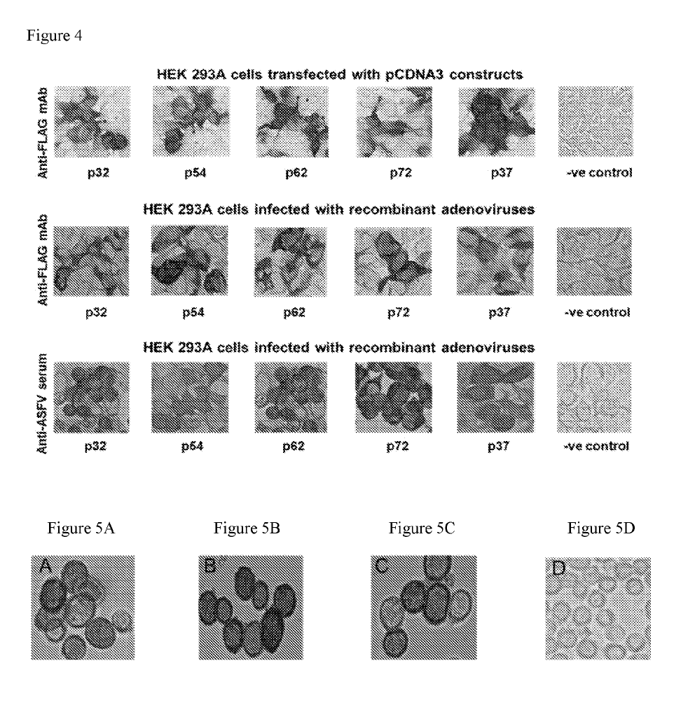

3. Evaluation of Protein expression by the pCDNA3 DNA constructs: The above selected clones of each one of the pCDNA3 constructs were tested for expression of the encoded ASFV antigens by immunocytometric analysis of Human Embryonic Kidney (HEK) 293 cell-transfectants probed with anti-FLAG and anti-HA monoclonal antibodies (mAbs), and with ASFV-reactive superpig serum (FIG. 4). Cells transfected with the empty pCDNA3 vector served as negative controls. The results showed that the pCDNA3 constructs expressed the encoded antigens as judged by positive staining with the anti-tag mAbs and the expressed antigens were authenticated by the positive staining with the ASFV-reactive superpig serum (FIG. 4). In addition, supernatants from the cell-transfectants were evaluated by ELISA using the ASFV-reactive superpig serum and shown to contain ASFV antigens (Data not shown).

4. Generation of Adenovirus, Baculovirus, BacMam, and Lentivirus plasmid DNA constructs: The best clone of each one of the pCDNA3 constructs mentioned above was selected to serve as template to PCR gene cassettes for the generation of recombinant adenovirus, baculovirus, BacMam, and lentivirus plasmid DNA expression constructs. Immunocytometric analysis and ELISA data was used to select the best clone based on protein expression efficiency as judged by staining with anti-tag mAbs and the ASFV superpig serum.

i) To generate adenovirus constructs, each antigen expression cassette was PCR amplified from the pCDNA3 constructs using flag-specific forward primer containing attB1 sequence and ha-specific reverse primer containing attB2 sequence (Invitrogen), cloned into pDonR-TOPO shuttle vector (Invitrogen), and positive clones were identified by PCR screening and validated by DNA sequencing. Selected recombinant pDonR constructs were then used to transfer cognate antigen expression cassette into the pAd adenovirus backbone by homologous recombination (Invitrogen). At least six clones of each pAd construct were selected and miniprep DNA was prepared for the generation of recombinant adenovirus. The workflow for gene cloning/screening/sequence validation is summarized in FIG. 3. Multiple clones of sequence-verified pDonR and respective pAd constructs encoding the ASFV p32, p54, p72, p62, p37, p150-I, and p150-II antigens were obtained.

ii) Recombinant baculovirus plasmid constructs were similarly generated as summarized in FIG. 3, but flag-specific forward and ha-specific reverse primers were used to PCR each gene from the pCDNA3 constructs. The PCR products were cloned into pFastBac-TOPO shuttle vector (Invitrogen) and positive recombinant pFastBac clones were identified by PCR screening and validated by DNA sequencing. One clone of each pFastBac construct was then used to generate Bacmid plasmid constructs (Invitrogen) encoding each antigen and positive clones were identified by PCR colony screening. At least six clones of each construct were selected based on PCR screening and miniprep DNA was prepared for baculovirus generation. Multiple clones of sequence-verified pFastbac and cognate Bacmid plasmid constructs encoding the ASFV p32, p54, p72, p62, p37, p150-I, and p150-II antigens (Table I) were obtained.

iii) The approach above ([ii]) was used to generate BacMam plasmid constructs, but each mammalian expression cassette was PCR amplified from each pCDNA3 construct using a forward primer (CMV Fwd) that incorporated the human CMV promoter and a reverse primer (TKpA Rev) that incorporated the TK polyadenylation and transcription termination sequences. The PCR products were used to generate recombinant pFastBac and Bacmid constructs as above. At least six clones of each Bacmid construct were selected and miniprep DNA was prepared for generation of BacMams. Multiple clones of sequence-verified pFastbac and respective BacMam plasmid constructs encoding the ASFV p32, p54, p72, p62, p3′7, p150-I, and p150-II antigens were obtained.

iv) To generate recombinant Lentivirus plasmid constructs the sequence validated pDonR clones encoding the ASFV p32, p54, p72, p62, p3′7, p150-I, and p150-II antigens (Table 1) were used to shuttle the genes into the pLenti7.3/V5-DEST vector in-frame to the V5-epitope tag using the Gateway technology (Invitrogen). This vector also contains EmGFP expression cassette for enhanced duo-expression of GFP protein. Positive clones were identified and validated by immunocytometric analysis as above. At least six clones of each pLenti construct were selected and miniprep DNA was prepared for generation of Lentivirus. Multiple clones of sequence-verified pLenti constructs encoding the ASFV p32, p54, p72, p62, p3′7, p150-I, and p150-II antigens (Table 1) were obtained.

### Example 2—Evaluation of Protein Expression by Constructs

In order to evaluate protein expression by the constructs encoding target antigens and validate the expressed antigen:

i) Protein expression by the pCDNA3 constructs encoding the ASFV p32, p54, p72, p62, p37, p150-I, and p150-II antigens was evaluated by immunocytometric analysis of HEK 293A cell transfectants and ELISA analysis of supernatants using the anti-tag mAbs and validated authenticity of the antigens using ASFV-reactive superpig serum as described in Example 1.

ii) The pAd constructs generated above were transfected into HEK 293A cells and the clones expressing the encoded antigen were identified by immunocytometric analysis of the cell-transfectants probed with the anti-tag mAbs and the ASFV superpig serum as above. Data from the immunocytometric analysis was used to select six lead clones of each construct for virus assembly (FIG. 4). Miniprep DNA was generated for each construct and an aliquot of each was frozen as stock for future use.

iii) The Bacmid constructs generated above (Example 1) were transfected into Sf9 insect cells to generate recombinant baculovirus. Positive clones were identified by immunocytometric analysis of the Sf9 insect cell-transfectants probed with the anti-tag mAbs and the ASFV superpig serum (FIG. 5). Supernatants from the transfected cells were recovered and tested for the presence of baculovirus. Data from the immunocytometric analysis was used to select 3 lead baculovirus clones expressing each antigen. One lead baculovirus for each construct was scaled up in T175 flask, tittered, and frozen in aliquots as working stock. Seven Bacmid constructs validate for generation of baculovirus expressing the ASFV p32, p54, p72, p62, p37, p150-I, and p150-II antigens were obtained. At least 3 baculovirus clones expressing each antigen were generated, stocks frozen and titered for bulk Baculovirus protein expression.

iv) The BacMam plasmid constructs generated above were transfected into Sf9 insect cells to generate recombinant BacMams. Assembly of the BacMam was tested by immunocytometric analysis of the cell-transfectants probed with baculovirus-specific mAb. Protein expression by the BacMams was tested by immunocytometric analysis of HEK 293A cells infected with the BacMam and then probed with anti-FLAG or anti-HA mAb and authenticity of the expressed ASFV antigen was validated using the ASFV superpig serum as above.

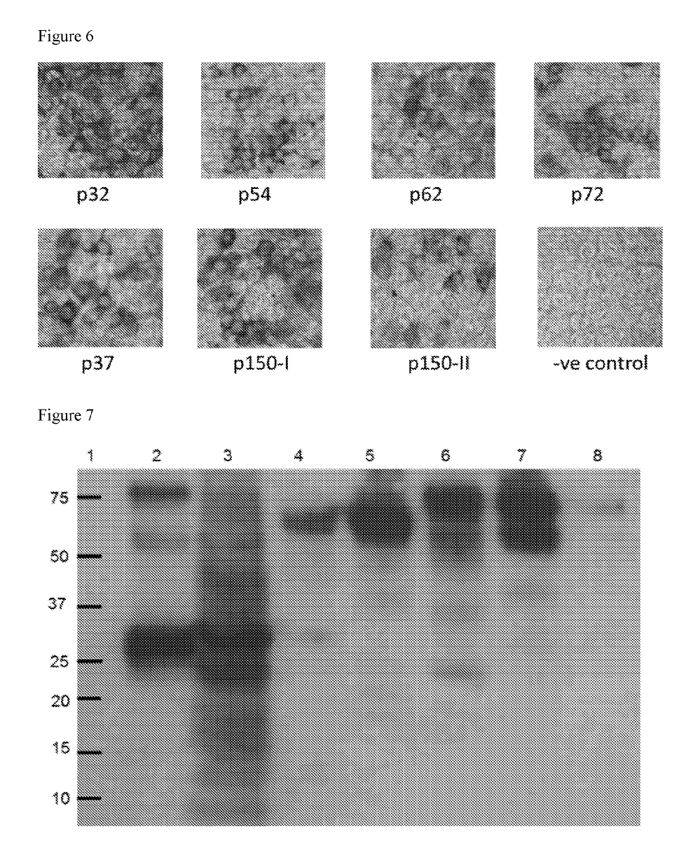

v) Protein expression by the pLenti-GFP constructs encoding the p32, p54, p72, p62, p3′7, p150-I, and p150-II antigens was tested by immunocytometric analysis of HEK 293A cell-transfectants probed with anti-V5 tag mAb and authenticity of the expressed ASFV antigen was validated using the ASFV superpig serum as above. Multiple clones of each construct were shown to express the encoded antigen (FIG. 6) and this outcome was consistent with antigen expression profiles shown in FIG. 4. One clone of each construct was selected based on immunocytometric analysis data and used to assemble recombinant Lentivirus according to the manufacturer's instructions (Invitrogen). Seven pLenti constructs (multiple clones) validated for protein expression and generation of Lentivirus expressing the p32, p54, p72, p62, p3′7, p150-I, and p150-II antigens were obtained.

### Example 3—Generation of Bulk Affinity Purified Recombinant Proteins

Bulk affinity purified recombinant proteins (p32, p54, p72, p62, p37; p150-I and p150-II) were generated and quality control tests using anti-tag mAbs and the ASFV-reactive superpig serum performed.

i) To generate recombinant proteins in mammalian cells, the pCDNA3 constructs encoding p32, p54, p72, p62, p37; p150-I and p150-II antigens had to be modified by adding an in-house optimized leader signal sequence, designated CD7, in-frame at the 5′ end of each gene for efficient protein secretion into the medium. Protein expression by miniprep DNA of the resultant constructs were screened by immunocytometric analysis and ELISA as above and the best performing clone of each construct was selected. Maxiprep DNA was prepared and quality control tested for protein expression. Pilot studies using HEK 293 Freestyle cell system (Invitrogen) showed that, only the pCDNA3CD7p62 construct gave sufficient protein yields and therefore, this construct and the expression system was used multiple times to generate p62 protein needs throughout this study. The expressed protein was affinity purified from the supernatants of transfected cells using anti-FLAG agarose beads (Sigma), ran on PAGE to evaluate purity, and validated by Western Blot analysis (FIG. 7).

ii) Baculoviruses encoding p32, p54, p72, p37; p150-I and p150-II were used for large scale protein expression using the High Five insect cell system (Invitrogen). Multiple batches of these antigens were generated because the baculoviruses encoding some of these antigens gave moderate to low yields. The expressed antigens were purified and tested as above (FIG. 7). Minipreps and maxipreps of the pCDNA3CD7-constructs encoding the secreted versions of p32, p54, p62, p′72, p3′7, p150-I, and p150-II antigens were obtained. Milligram quantities of HEK293-expressed p62 and Baculovirus-expressed p32, p54, p72, p37, p150-I, and p150-II antigens were generated and used to execute various tasks as described below.

### Example 4—Scale Up of pCDNA 3 Constructs

PCDNA3 constructs were scaled up; adenoviruses, Lentiviruses, and BacMams expressing the ASFV targets (p32, p54, p′72, p62, p37; p150-I and p150-II) were assembled and scaled up; and quality control tests were performed using anti-tag mAbs and the ASFV-reactive superpig serum.

i) Selected clones of each one of the pCDNA3 plasmid DNA constructs expressing the ASFV targets (p32, p54, p72, p62, p37; p150-I and p150-II) were scaled up to generate Maxiprep DNA and protein expression was validated by immunocytometric analysis as above. The generated DNA was used to transfect autologous skin fibroblasts for use as cytotoxic T lymphocyte (CTL) targets. Maxipreps of pCDNA3 constructs expressing p32, p54, p′72, p62, p37, p150-I, and p150-II were obtained. The empty pCDNA3 vector was also amplified to serve as a negative control.

ii) Lead pAd DNA constructs expressing the p32, p54, p72, p62, p37, p150-I, and p150-II antigens were used to assemble recombinant replication-incompetent adenoviruses by transfecting HEK 293A cells with Pac I-digested miniprep DNA using a well-established protocol (Invitrogen). Six clones of each construct were used to assemble recombinant adenoviruses and the best clones were selected based on protein expression as judged by immunocytometric analysis using anti-tag mAbs and superpig serum (FIG. 4). Each one of the selected clone was amplified in T75 tissue culture flask and used as working stock to generate bulk adenovirus by infecting 40 T175 flasks for each construct. The bulk viruses were tested for protein expression and antigen authenticity was validated by immunocytometric analysis as above and by Western Blotting (data not shown). Following titer determination, the presence of replication-competent adenovirus was tested by evaluating replication competence in non-complementing cell lines and primary cells. All the bulk virus preparations were shown to be replication incompetent. Large scale (10¹¹-10¹²) replication-incompetent recombinant adenoviruses validated for expression of p32, p54, p72, p62, p37, p150-I, and p150-II. In addition, bulk replication-incompetent recombinant adenovirus, Adeno-Luciferase (AdLuc), was generated to serve as a negative control.

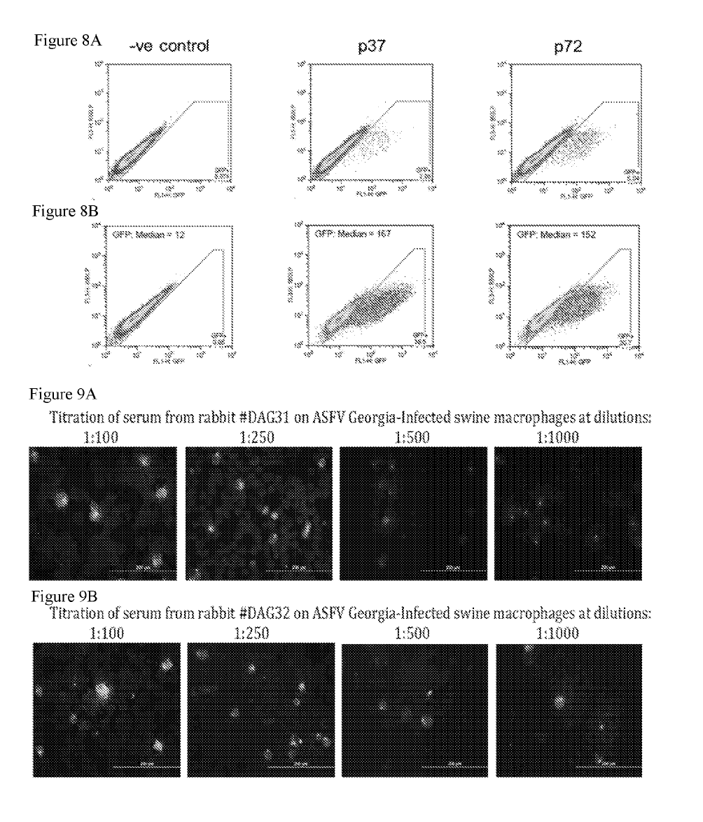

iii) Lead pLenti DNA constructs expressing the p32, p54, p72, p62, p37, p150-I, and p150-II antigens were used to assemble recombinant Lentiviruses by co-transfecting HEK 293FT producer cells with packaging mix using a well-established protocol (Invitrogen). Several clones of each construct were used to assemble recombinant lentiviruses and the best clones were selected based on protein expression as judged by immunocytometric analysis using anti-V5 tag mAb and superpig serum. One selected clone of each construct was used to generate bulk virus by repeated co-transfection of HEK 293FT producer cells as above in petri-dishes. The bulk viruses were tested for protein expression by immunocytometry as above and assembly of virus was confirmed by infecting HEK 293A followed by evaluation of GFP expression by flow cytometry (FIG. 8). Protein expression was also evaluated by flow cytometric analysis of infected primary porcine fibroblasts. Recombinant Lentiviruses validated for expression of p32, p54, p′72, p62, p37, p150-I, and p150-II were obtained. However, some recombinant Lentiviruses (e.g. p54, p150-I and -II) generated low yields and a pilot study to compare protein expression efficiency in primary porcine fibroblasts infected with either the recombinant Lentivirus (at graded MOIs) or transfected with the pCDNA3 plasmid DNA constructs showed that the latter performed much better than the former and the transfected fibroblast monolayers were much healthier.

### Example 5—Generation and Characterization of Monoclonal and Polyclonal Antibodies Against Two Asfv Proteins

Anti-p54 and anti-p62 rabbit polyclonal sera were generated and reactivity against ASF virus was evaluated.

i) Two rabbits, designated DAG31 and DAG32 were immunized with affinity purified recombinant p62 protein following routine immunization protocols through custom service (R. Sargeant, Ramona, Calif.). Sera demonstrated specific staining in IFA using ASFV-infected swine macrophages (FIGS. 9A and 9B).

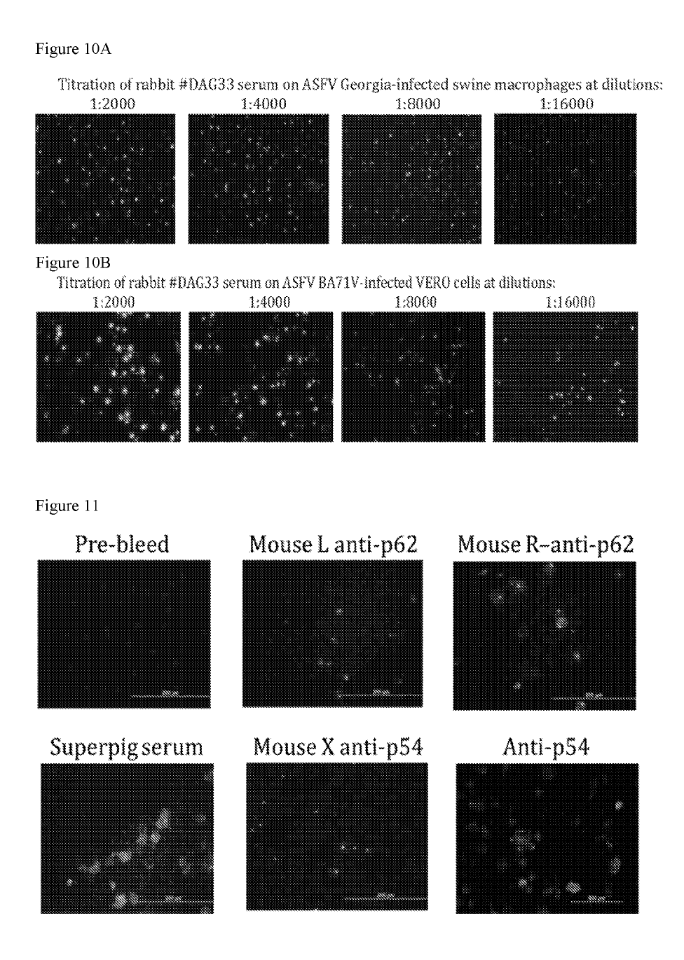

ii) Two rabbits, designated DAG33 and DAG34 were immunized with affinity purified recombinant p54 proteins as above and IFA testing of the sera demonstrated specific staining against ASFV-infected swine macrophages and VERO cells (FIGS. 10A-10D).

### Example 6—Generation and Characterization of Anti-P54 and Anti-P62 Monoclonal Antibodies

i) Immunization of mice: Mice were immunized with either affinity purified p54 or p62 recombinant protein for monoclonal antibody production. Sera from these immunized mice were shown to recognize their respective antigen expressed by 293A cells transfected with the pcDNA construct, and by 293A cells infected with adenovirus expressing p54 or p62. In addition, IFA performed on pre-fusion mouse sera were shown to react specifically with ASFV-infected macrophages (FIG. 11).

ii) Generation of hybridomas: Splenocytes were harvested and fused with Sp2/0 myeloma cells for hybridoma production. Hybridoma cell culture supernatants were screened on 293A cells transfected with the pCDNA construct expressing either p54 or p62. Selected hybridoma cell culture supernatants were further tested on ASFV BA71-infected and mock-infected VERO cells (Table 2). Selected anti-p54 and anti-p62 hybridomas were cloned by limiting dilution in a 96-well format (Table 3). Positive subclones were then validated by immunocytometric analysis on 293A cells transfected with either the p54 or the p62 DNA construct, and then by Western blotting against cell lysates similarly transfected 293A cells. Three of the subclones from anti-p62 clone 3F2 were detected by Western Blot. Frozen aliquots of these three subclones were cryogenically stored and some shipped to DHS.

iii) Generation of hybridoma subclones: The initial p54 parental anti-p54 hybridomas tested positive by ELISA and were also IFA positive on ASFV-infected cells (DHS) and were subcloned. The subclones' supernatants tested positive by ELISA and Western blot against the recombinant p54 protein. Supernatant was sent to DHS for IFA validation but subclones were negative. Therefore, an additional electro-fusion was performed with anti-p54 mouse splenocytes. ELISAs (using crude baculovirus supernatant to coat ELISA plates) and immunocytometric analysis were used to screen for positive parent hybridomas. Eight anti-p54 parental hybridomas tested positive by both ELISA and immunocytometry: 1B8, 1C2, 2C4, 2D9, 2E4, 2E7, 2G4, and 3B12. Parental anti-p54 hybridomas 1C2 and 2C4 were cloned by limiting dilution in a 96-well format. The 2C4 subclones did not survive, while the 1C2 subclones showed good viability. Twenty-one anti-p54 subclones from the parent hybridoma 1C2 were positive by ELISA and by immunocytometry, and were cryogenically stored.

### Example 7—Induction of an ASFV-Specific Immune Response in Pigs with Recombinant Adenoviruses Expressing the Lead Targets

A. Piglets Immunization with Graded Doses of a Cocktail of the Adenoviruses Expressing the Lead Targets Formulated with Defined Adjuvants.

i) Twenty weaned piglets (˜30 lbs) were acquired and during quarantine period, they were vaccinated against defined pathogens to meet institutional requirements. Skin biopsies were taken from each piglet and used to establish fibroblast cell lines to serve as autologous antigen presenting cells (APCs) in in vitro CTL readouts. In addition, ConA PBMC blasts were generated and frozen as backup autologous APCs. Fast growing fibroblasts were frozen, and slow growers or those that were struggling to grow were immortalized (by infecting with Lentivirus expressing Large T antigen) to fast-track growth.

ii) Pilot studies were conducted to optimize transfection efficiency of the porcine fibroblasts and up to 40% efficiency was achieved. Antigen expression by the transfected cells was shown to be much better than infection with recombinant Lentiviruses. Some fibroblasts were tested for ⁵¹Cr labelling-release in preparation for CTL assays.

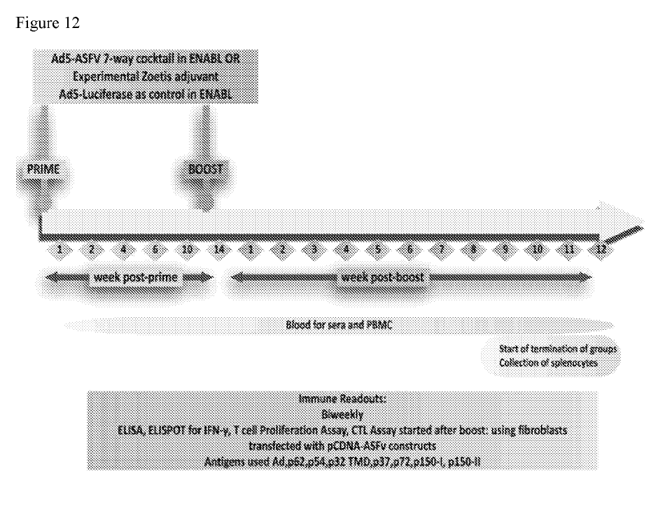

iii) The piglets were randomly divided into four groups (n=5) and immunized (2 mLs×3 i.m. sites) with a cocktail of the seven recombinant adenoviruses expressing the p32, p54, p72, p62, p3′7, p150-I, and p150-II ASFV antigens formulated in defined adjuvants (Table 5). This protocol was also used for boosting (FIG. 12).

B. Piglet Monitoring to Document Localized and/or Systemic Adverse Effects.

i) Post-priming: Following inoculation of the adenovirus cocktail, both the 10¹⁰ and the 10¹¹ adenovirus doses/adjuvant formulations (Table 5) were well tolerated and no adverse systemic effects or injection site reaction were record (Table 6).

ii) Post-boosting: A day after boosting (Table 5 and FIG. 12), pigs in groups 1-3 were depressed and had reduced appetite. In addition, some had swelling at the injection site. Pigs in group 4 were active but all had a pink spot at the injection site. On day 2 post-boost, all pigs in groups 1-3 were depressed and had reduced appetites. Some of these animals were given Banamine to control fever (>103° C.). However, by the 3rd day post-boost, all the pigs were active, healthy and with good appetite, and remained so for the rest of the study period (Table 6). Overall, these observations are indicative of a well-tolerated immunogen. Therefore, the experimental Ad-ASFv cocktail vaccine formulations were well tolerated post-prime and induced transient fever/inoculation site swelling in some pigs post-boost.

C. Evaluation of ASFV Antigen-Specific Antibody Responses Post-Prime and Post-Boost.

Antigen-specific antibody responses were monitored starting on day 7 post-priming and tracked for 14 wks when the pigs were boosted and post-boost immune profiles were monitored for 8 wks.

i) Sero-conversion and isotype switching: Antibody responses were evaluated by ELISA using plates coated with affinity purified recombinant ASFV antigens generated as described above. Post-prime sera were tested at 1:100 dilutions. Seven days post-priming, all the pigs inoculated with the 10¹⁰ or 10¹¹ adenovirus dose had sero-converted and developed

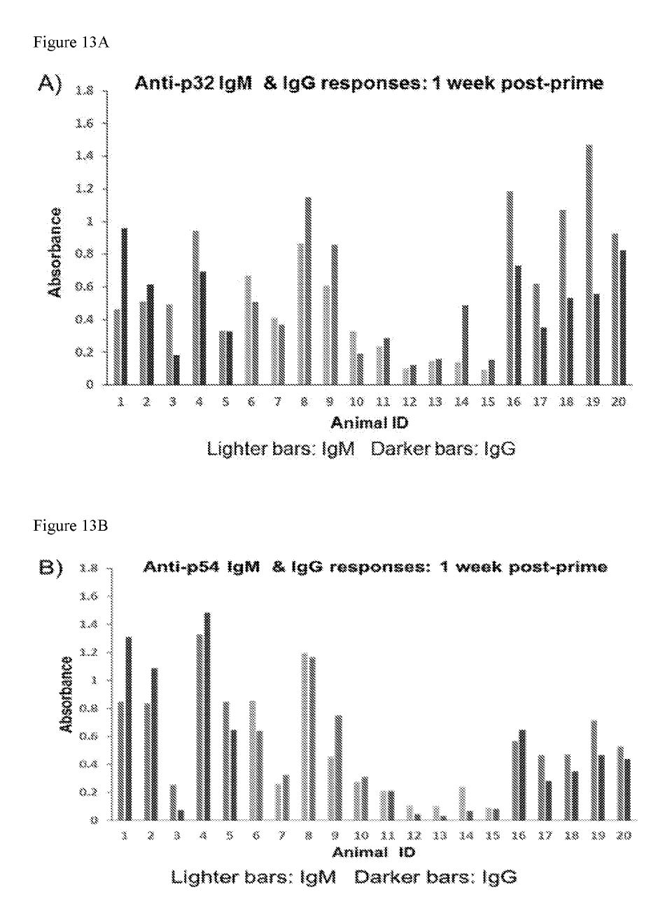

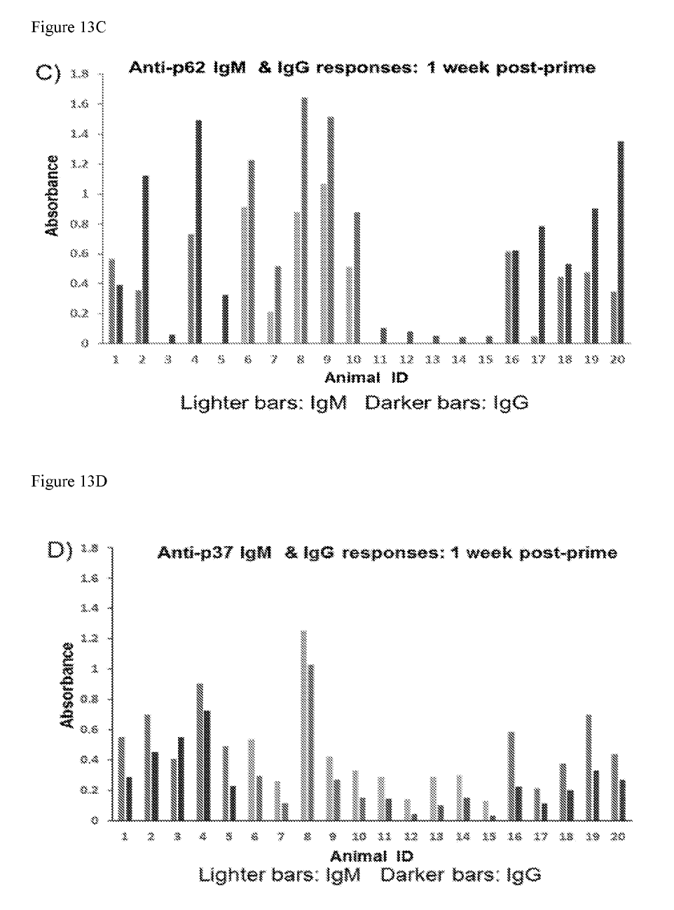

ASFV antigen-specific antibodies. More importantly, most pigs had isotype-switched and were generating ASFV antigen-specific IgG antibodies. Data for p32, p54, p62, and p37 are shown in FIG. 13A-D. The antibody responses at 7 days post-priming showed that the cocktail containing the seven recombinant adenoviruses expressing the p32, p54, p72, p62, p3′7, p150-I, and p150-II ASFV antigens rapidly induced antibody responses against each antigen. Importantly, there was significant isotype switching in the majority of the pigs. This is a significant outcome given that, a vaccine against a fatal animal disease, such a as ASFV, ideally should be able to rapidly induce robust immune responses in the face of an outbreak. It is notable that both 10¹⁰ and 10¹¹ doses induced similar levels of antibody responses. At a glance, post-prime responses did not reveal any difference in regards to the adjuvant used. These data demonstrate rapid induction of antibody responses against multiple antigens with a single dose immunization.

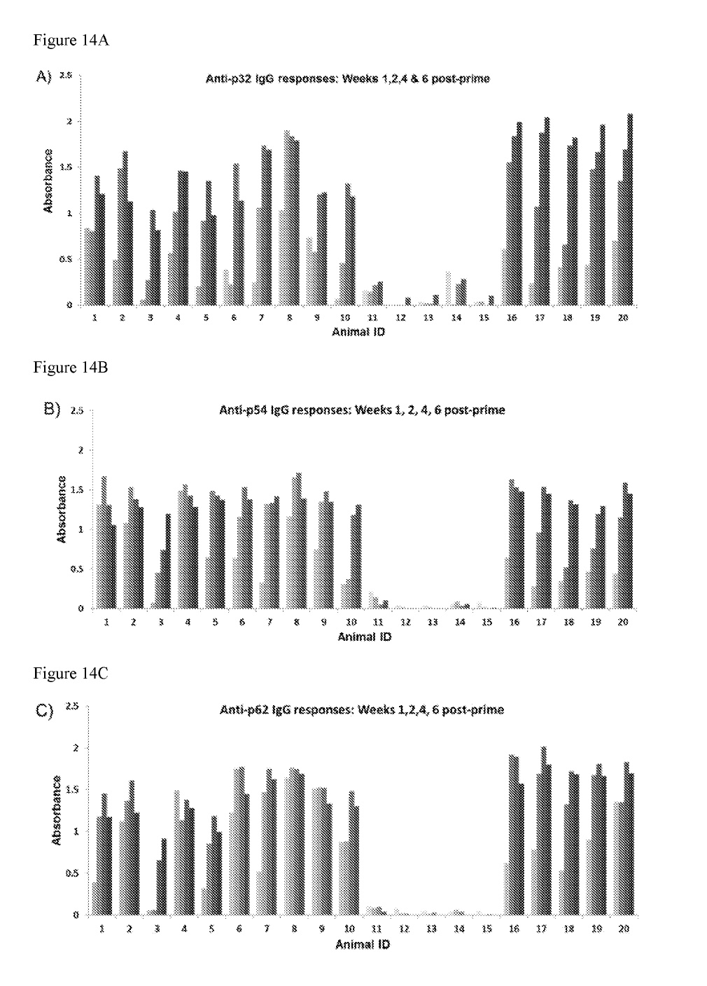

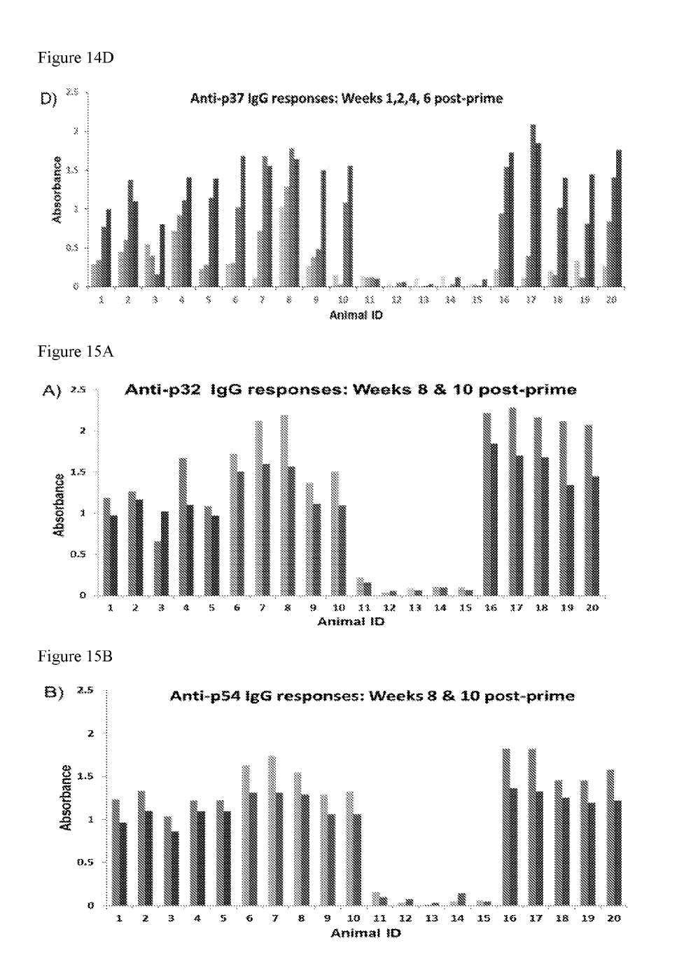

ii) ASFV antigen-specific Antibody profiles post-priming: Following priming, antigen-specific IgG responses were monitored biweekly. Antibody responses were evaluated by ELISA as above and post-prime sera were tested at 1:100 dilutions. In all the three treatment groups, but not the sham treatment, antibody responses against all the antigens increased during the first six weeks. Data for p32, p54, p62, and p37 are shown (FIGS. 14A-14D).

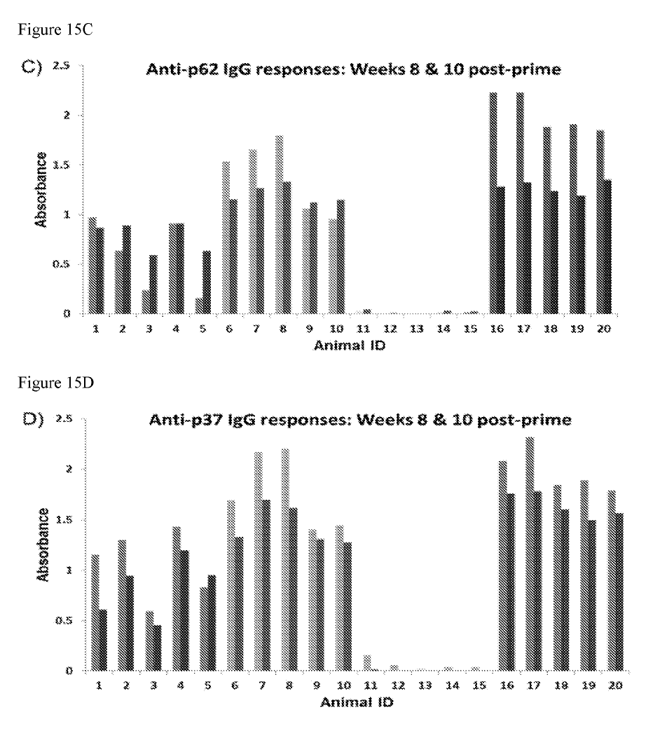

iii) ASFV antigen-specific IgG responses peaked 8 weeks post-priming: Tracking antibody response over time allowed monitoring of adenovirus-specific antibody profiles to determine a time point at which anti-vector titers declined to allow homologous boost with the priming cocktail. Antibody responses were evaluated by ELISA as above and post-prime sera were tested at 1:100 dilutions. In all the three treatment groups, but not the sham treatment, antibody responses against all the antigens peaked around 8 weeks post-priming and then started to decline at 10 weeks post-priming. Data for p32, p54, p62, and p37 are shown (FIGS. 15A-15D).

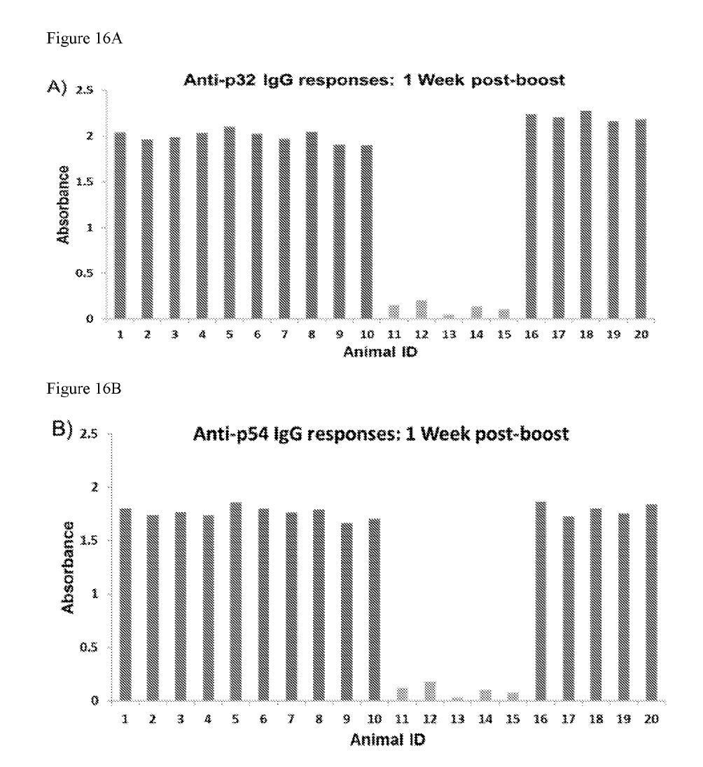

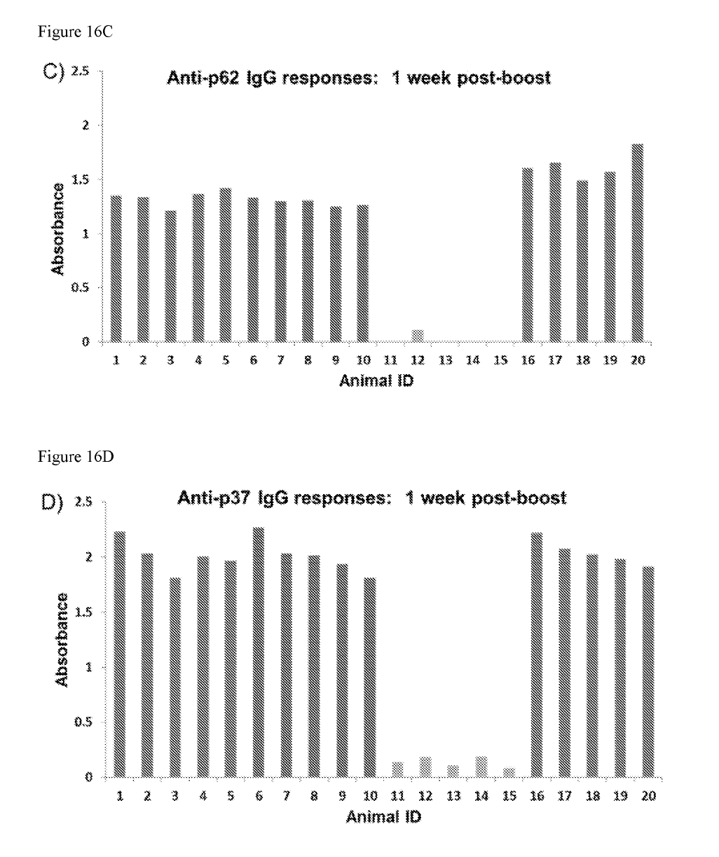

iv) Antigen-specific IgG responses post-boost: Pigs were boosted 14 weeks post-priming with the cognate priming cocktail and dose (Table 2). Antibody responses were evaluated by ELISA as above. In all the three treatment groups, but not the sham treatment, there was strong recall IgG antibody responses against all the antigens. Data for p32, p54, p62, and p37 are shown (FIGS. 16A-16D). It was determined that post-boost titers were >1:100,000 (data not shown).

D. Verification that the Induced Antibodies Recognize Actual ASF Virus and Antigens.

Indirect Immunofluorescence Antibody Assay (IFA) and Western Blotting were used to confirm whether antibodies induced by the experimental Ad-ASFv cocktail immunogens could recognize the actual ASF virus and ASFV antigens, respectively.

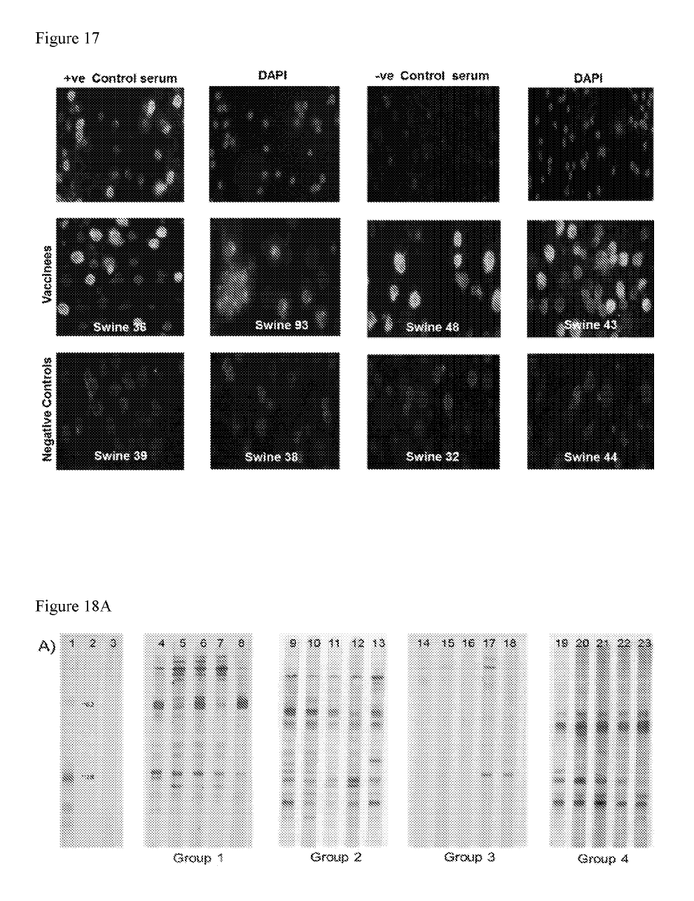

i) IFA outcome: Sera from 1 week post-boost were tested at 1:200 dilutions using primary swine macrophages infected with the ASFV George 2007/1 isolate. Superpig serum (1:500) was used as the positive control, whereas normal pig serum (1:200) was used as the negative control. Sera from all the three treatment groups, but not the sham treatment, strongly recognized the ASF virus (FIG. 17 and Table 7). All the vaccinated pigs had strong IFA signal against primary swine macrophages infected with the ASFV George 2007/1 isolate but not the sham controls. The overall IFA results are summarized in Table 3. Selected sera were titrated and shown to be >1:900 (data not shown). The IFA data demonstrate that the adeno-ASF cocktail induced authentic ASFV-specific antibody responses.

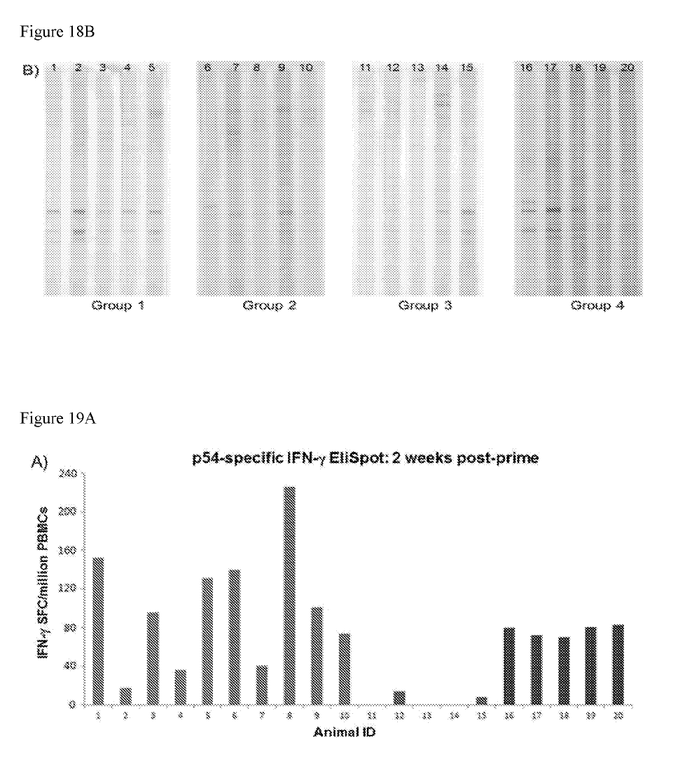

ii) Western Blot sera analysis: The sera from 1 week post-boost were tested by Western blotting at 1:50 dilutions using lysates from Vero cells infected with the ASFV George 2007/1 isolate. Superpig serum (1:10,000) was used as the positive control whereas normal pig serum (1:200) was used as the negative control. Sera from all the three treatment groups, but not the sham treatment, strongly recognized the ASFV antigens (FIG. 18A). Western blot conducted using sham-infected Vero cell lysate served as the antigen control to evaluate background reactivity against host cell antigens (FIG. 18B). These results demonstrated that the immunized pigs mounted antigen-specific antibody responses to all the antigens in the cocktail. The antibody responses underwent strong recall upon boost and more importantly, the antibodies strongly recognized the ASF virus and viral proteins.

E. Evaluation of ASFV Antigen-Specific T Cell Responses Post-Prime and Post-Boost.

Antigen-specific T cell responses were monitored starting on day 14 post-priming and tracked for 14 weeks when the pigs were boosted and post-boost immune profiles were monitored for 8 weeks (FIG. 12).

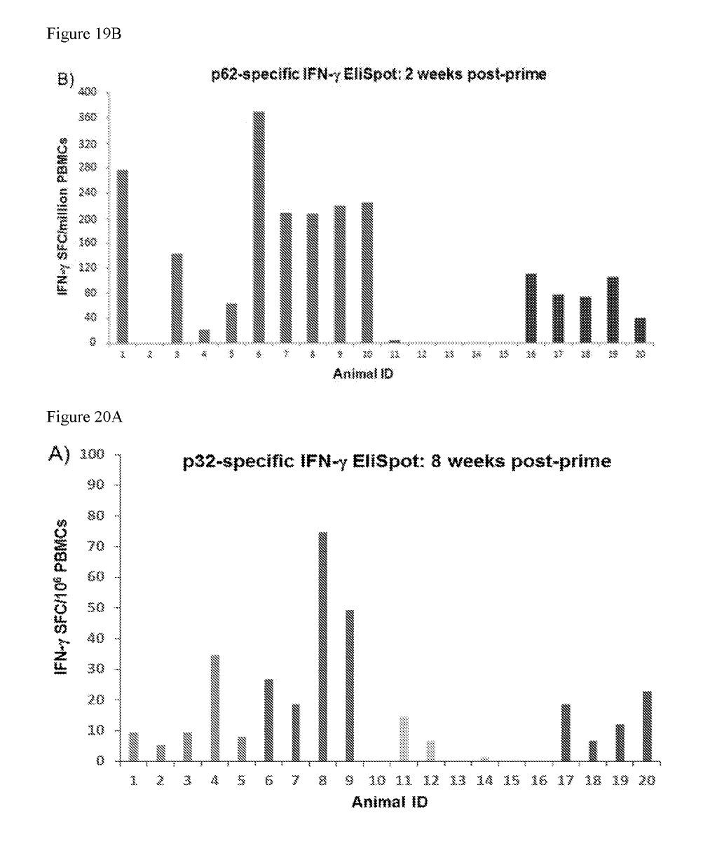

i) Ad5-ASFv cocktail primed IFN-γ-secreting cells: ELISPOT assays were used to evaluate and quantify antigen-specific IFN-γ-secreting cells in whole peripheral blood mononuclear cells (PBMCs). Data is presented as Spot Forming Cells (SFC)/10⁶ PBMCs. Two weeks post-priming, antigen-specific IFN-γ-secreting cells were detected in most of the pigs inoculated with the 10¹⁰ and the 10¹¹ adenovirus dose, but not the negative controls. Overall, there was no distinct difference in responses among the dose and adjuvant treatment groups tested. Data for p54 and p62 are shown (FIGS. 19A-19B).

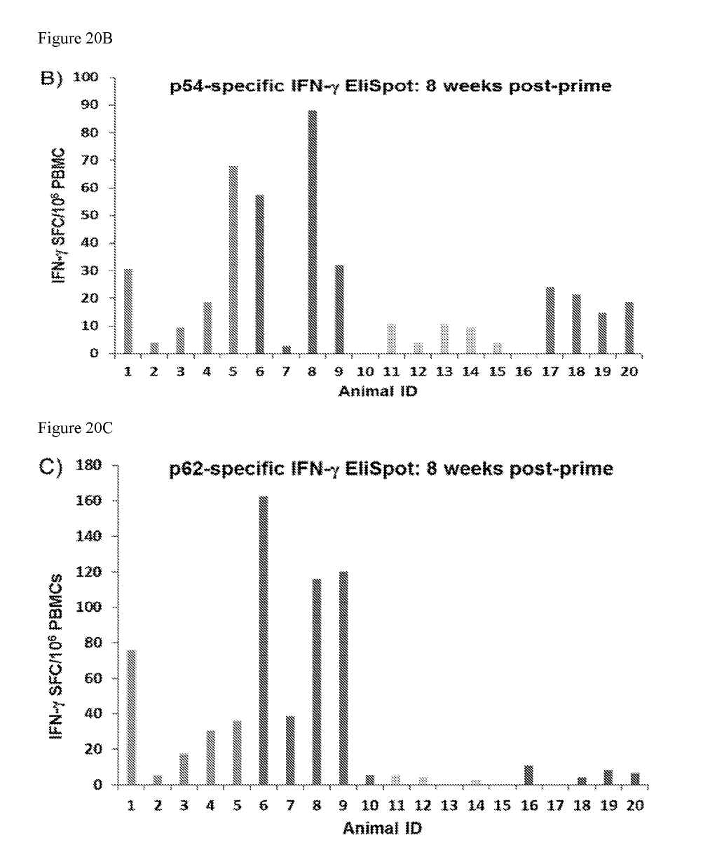

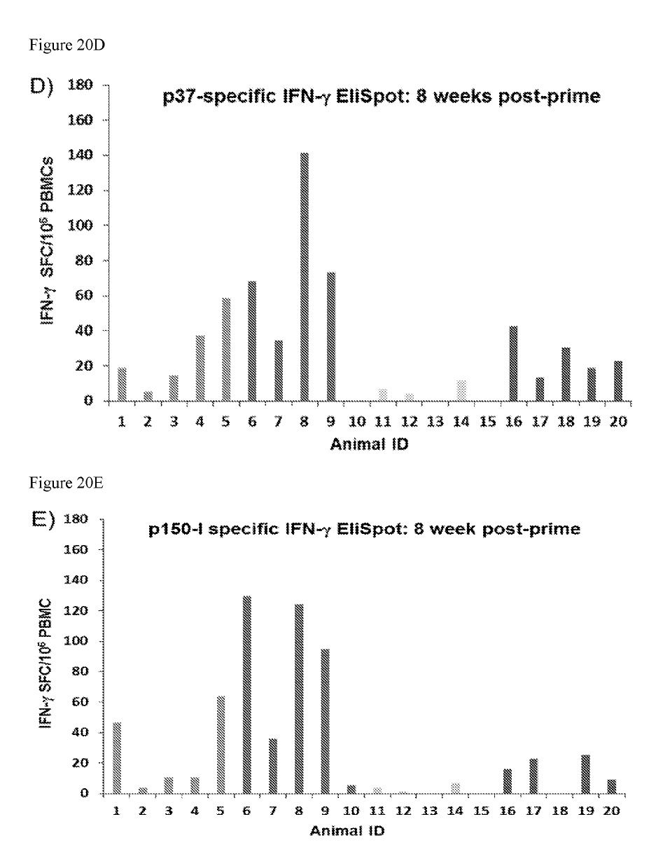

ii) IFN-γ responses eight weeks post-priming: ELISPOT assays were used to track antigen-specific IFN-γ-secreting cells in whole PBMCs over time. Eight weeks post-priming, when antibody profiles plateaued, significant antigen-specific IFN-γ-secreting cells were still detectable in most of the pigs inoculated with the 10¹⁰ and the 10¹¹ adenovirus doses, but not the negative controls. Compared to the other treatments, group 2 vaccinees had relatively higher levels of antigen-specific IFN-γ-secreting cells. This outcome is inconsistent with antibody responses documented at the same time post-priming, whereby antibody responses were similar among the treatment groups (FIG. 13). Data for p32, p54, p62, p37, and p150-I are shown (FIGS. 20A-20E).

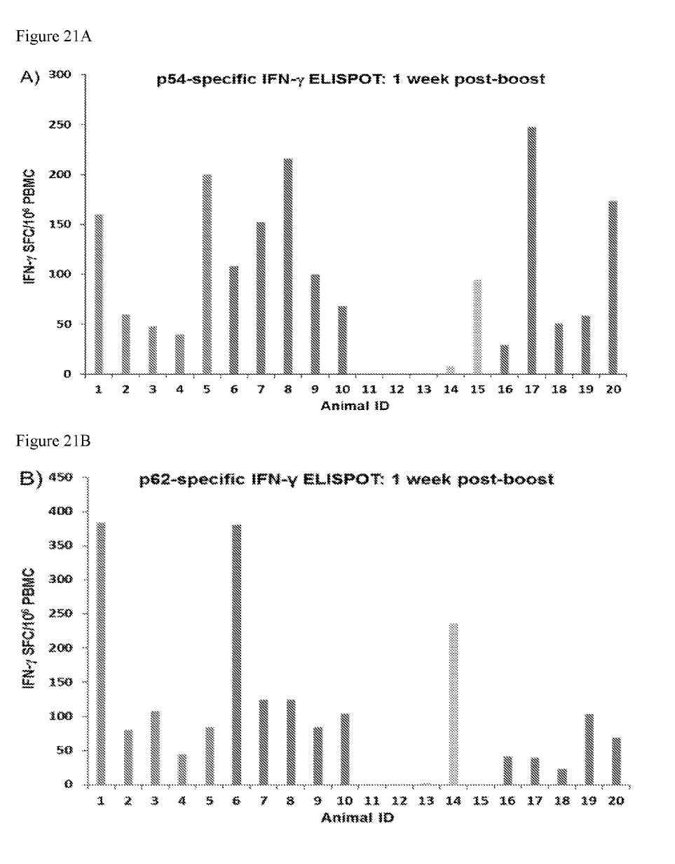

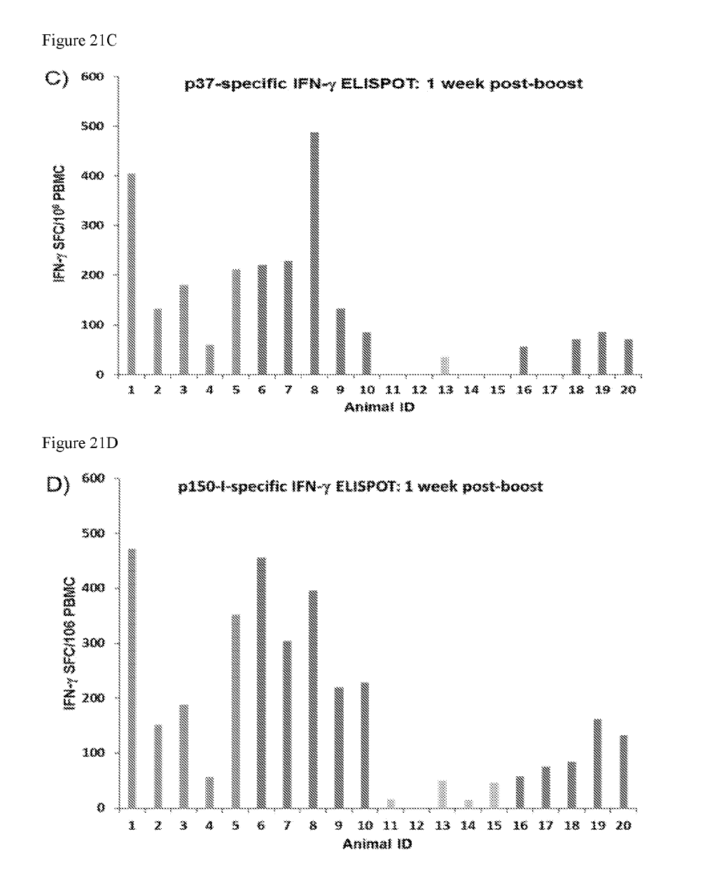

iii) Recall IFN-γ responses: Following boosting with the priming immunogen and dose (Table 5 and FIG. 12), strong antigen-specific IFN-γ-secreting T cell recall responses were detected by ELISPOT assay one week post-boost. Data for p54, p62, p37, and p150-I are shown (FIGS. 21A-21D).

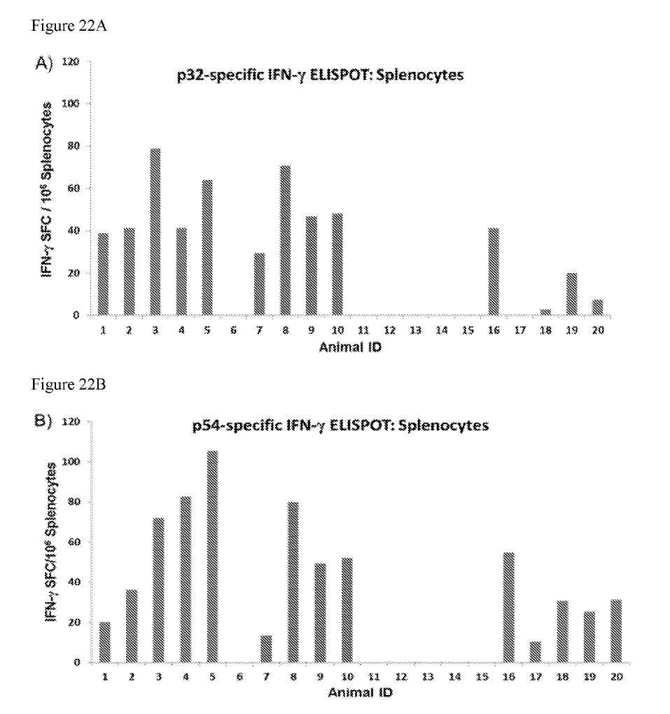

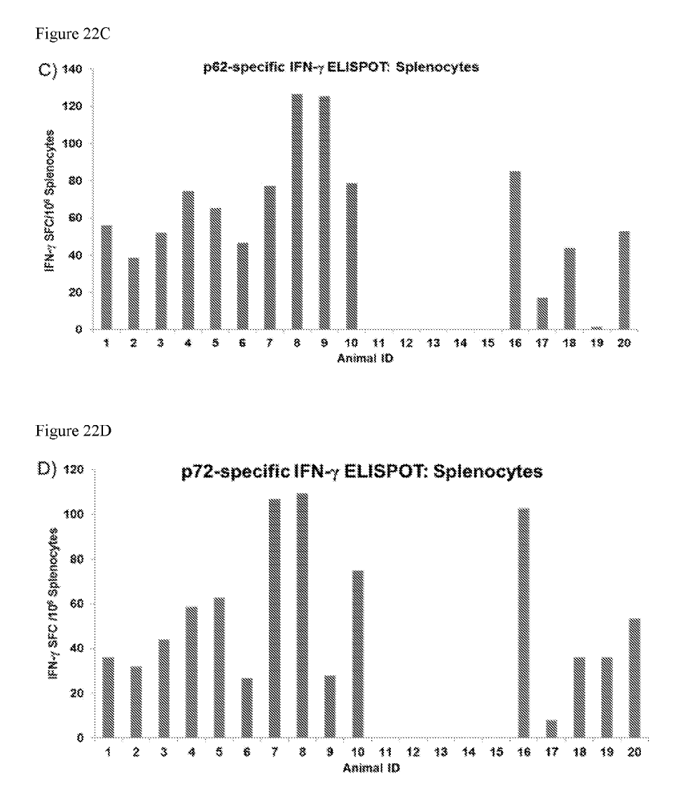

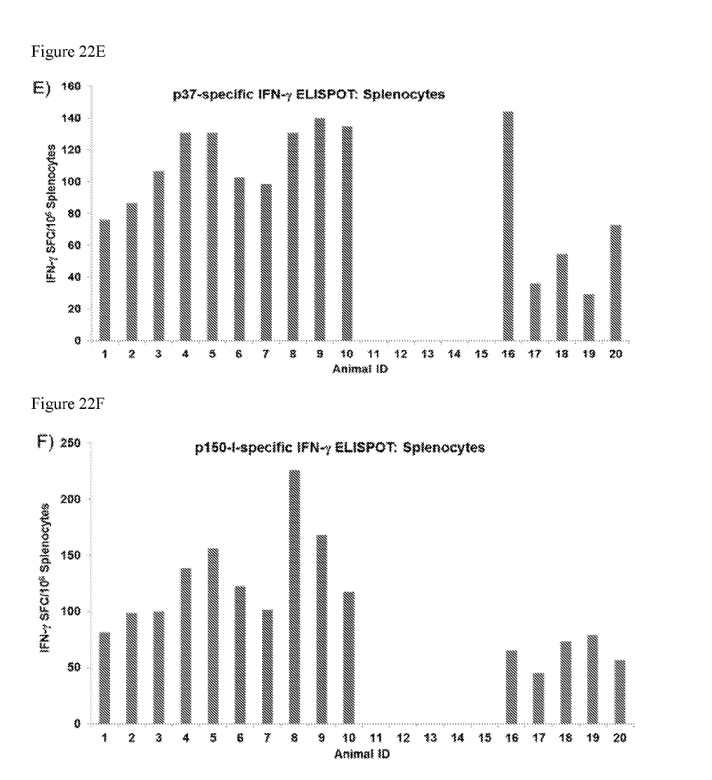

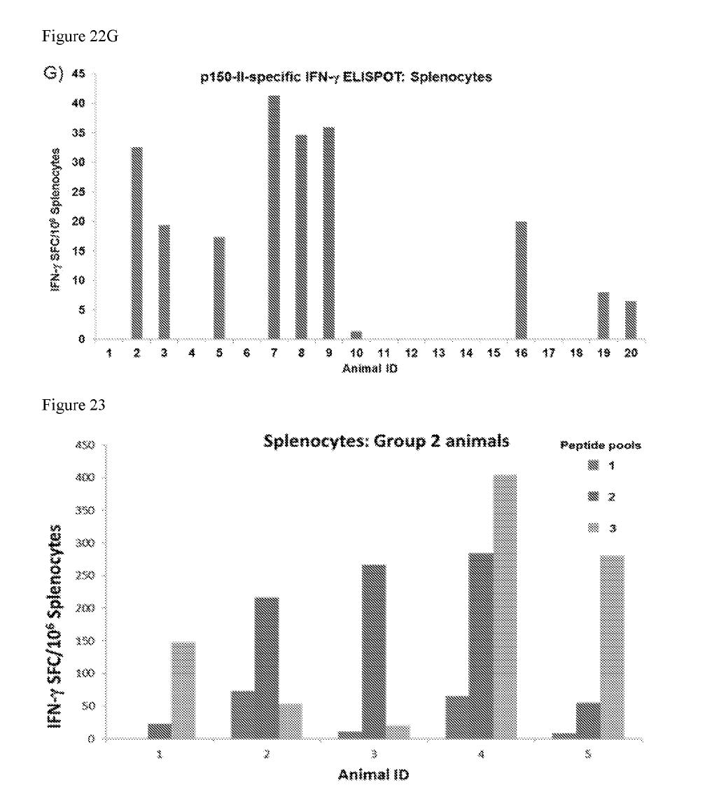

iv) Memory IFN-γ-producing cells in spleen: Two months post-boosting, the experiment was terminated and antigen specific memory cells in spleens were evaluated by ELISPOT assay. Antigen-specific IFN-γ+ T cell responses were detected. Data for all antigens are shown (FIGS. 22A-G).

v) Ad-ASFv immunized pigs recognized predicted SLA-1 binding peptides: IFN-γ ELISPOT assay was used to test whether splenocytes from the pigs immunized with the Ad-ASFV experimental cocktail vaccine could recognize predicted SLA-1 binding peptides from Georgia ASFV antigens. Three peptide pools (20 peptides/each) tested stimulated strong responses (FIG. 23).

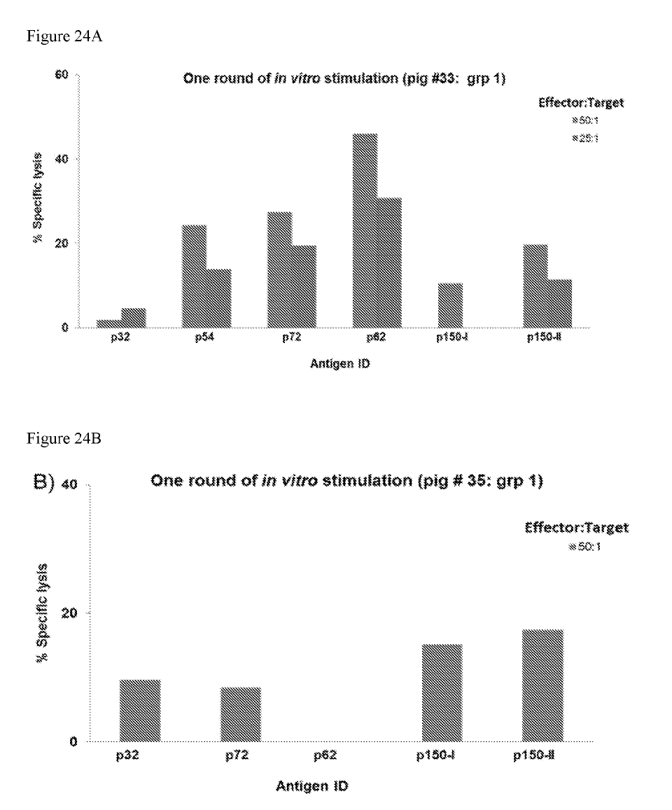

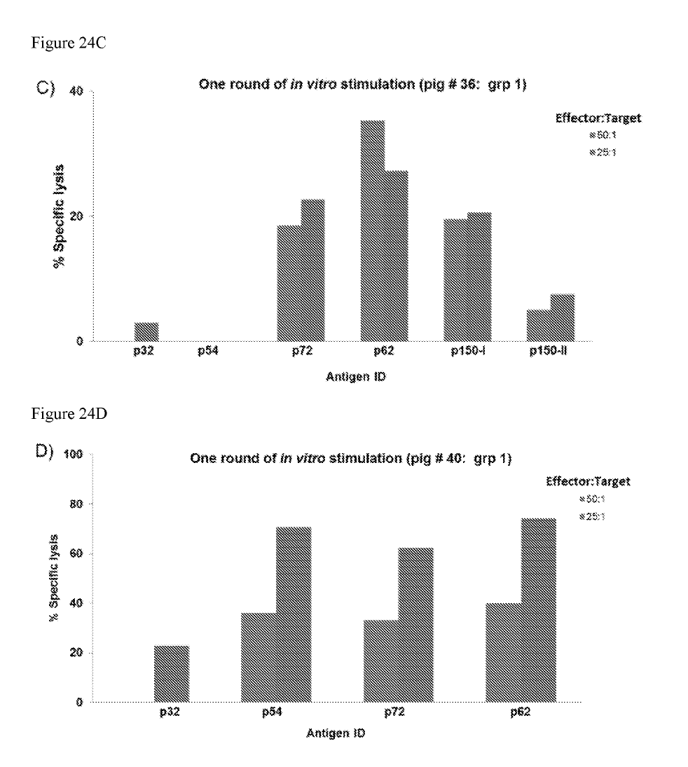

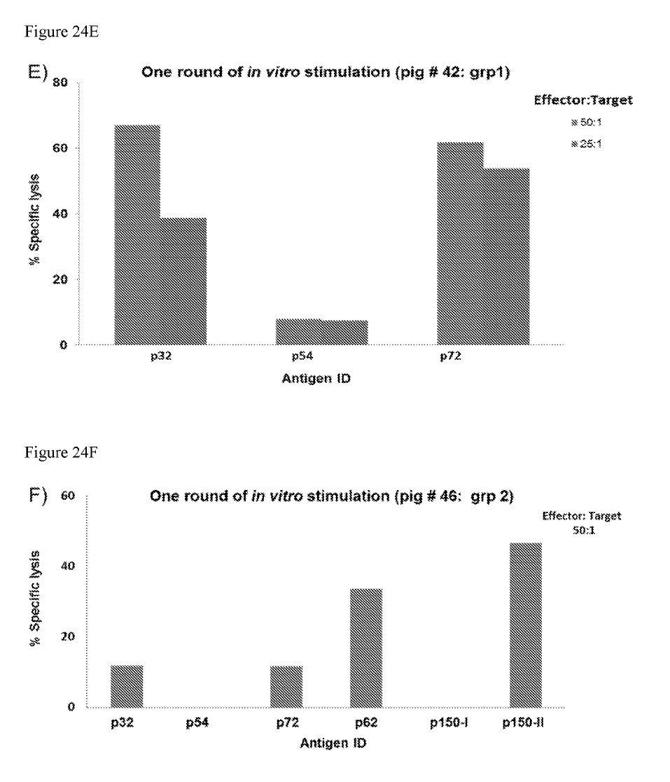

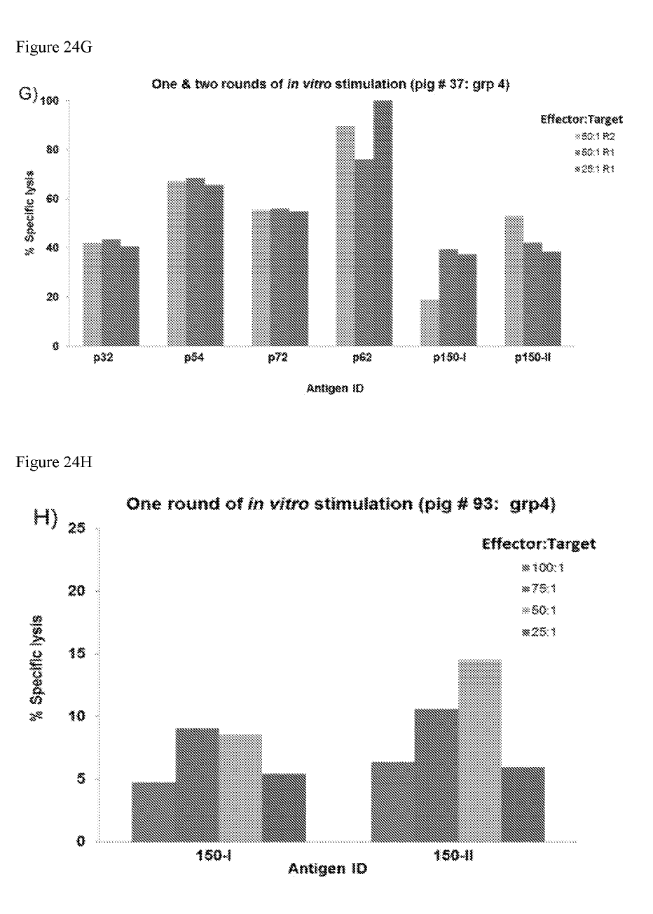

vi) Ad5-ASFv cocktail primed ASFV antigen-specific Cytotoxic T lymphocytes (CTLs): Autologous skin fibroblasts were established from each pig at the start of the study and used to evaluate ASFV antigen-specific CTLs. Following boosting, autologous monocytes infected with the Ad5-ASFv cocktail were used as antigen presenting cells (APCs) to stimulate peripheral blood mononuclear cells to enrich for ASFV antigen-specific T cells. The bulk cultures were assayed for antigen-specific CTL activity at defined effector-target ratios using ⁵¹Chromium-labelled autologous fibroblasts transfected with the pCDNA construct expressing cognate antigen. Data is presented as net % target killing (background counts for each test have been subtracted). Antigen-specific target killing was detected in the immunized pigs and data for various antigens at defined effector-target ratios are shown (FIGS. 24A-24H). The data above (FIGS. 19-24) demonstrate that the immunized pigs mounted strong IFN-γ secreting T cells detectable as early as two weeks post-priming (earliest time tested) and eight weeks post-priming. Overall, the immunized pigs, but not the negative controls, responded to all the antigens and this outcome mirrors IgG responses (FIGS. 13-18). The primed IFN-γ responses underwent strong recall response upon boosting and in addition, strong IFN-γ secreting T cells were detected in splenocytes. The primed IFN-γ-secreting T cells were shown to recognize predicted SLA-1 binding peptides from the Georgia ASFV antigens and this outcome strongly indicates that the T cells primed by the Ad-ASFv cocktail immunogen are ASFV-specific (FIG. 23). More importantly, the experimental vaccine primed antigen-specific CTL responses detectable after one round of in vitro restimulation (FIG. 24). The heterogeneous CTL responses are consistent with expected outcomes from the outbred pigs used in this study. However, the CTL responses detected will need to be validated for killing of targets infected with the actual ASF virus. Taken together, the strong IFN-γ T cell and CTL responses support the hypothesis that adenovirus-encoded multi-antigen cocktail is capable of inducing T cell responses against each antigen in the immunogen and this supports development of a multi-antigen vaccine approach for ASFV.

### Example 8—Generation of Constructs Encoding ASFV Antigens and Chaperones

Two promising vaccine candidates, namely B119L and B646L, were selected for development of a prototype subunit vaccine. However, B119L and B646L are poorly expressed by live vectors that we had previously evaluated. To improve expression of B119L and B646L antigens using live vectors, natural chaperones A151R and B602L were co-expressed. A151R is a natural chaperone for B119L and significantly improved expression. A151R is also involved in the expression of B646L. B602L is a natural chaperone for B646, is highly expressed in live vector, and is required for the processing of other ASFV proteins needed for virus assembly. Furthermore, deletion of B602L severely alters viral assembly.

Briefly, the amino acid sequences of the A151R, B119L, B602L and B646L proteins from all the currently characterized ASFV isolates were compared, and consensus amino acid sequences were identified and selected for this study. The amino acid sequence of each antigen was modified to add a tag (FLAG) at the end to allow tracking protein expression using a commercially available antibody (anti-FLAG) and affinity purification of recombinant proteins. The resultant amino acid sequences were used to design synthetic genes optimized for protein expression in swine cells and the genes were synthesized commercially. The synthetic genes were used to generate recombinant replication-incompetent adenoviruses designated AdA151R, AdB119L, AdB602L and AdB646L. Protein expression by these recombinant viruses was tested using the anti-FLAG tag antibody and authenticity was validated using ASFV-specific immune serum from a pig that had been immunized with the ASF virus. In addition, the synthetic genes were used to generate recombinant baculoviruses which were used to express A151R, B119L, B602L and B646L recombinant proteins needed for evaluating antibody and T cell responses after immunization of pigs.

Generation of Codon-Optimized Genes and Design of Expression Cassette.

The ASFV A151R, B119L, B602L, and B646 amino acid sequences from all the currently sequenced genomes were aligned and using the George 2007/1 as the reference sequence, consensus amino acid sequences were identified and selected for this study. In most cases where there was no consensus, Georgia 2007/1 amino acid sequences were selected. The amino acid sequence of each antigen was modified to add, in-frame, a FLAG- and HA-tag at the N- and C-termini, respectively, to generate an expression cassette for each antigen. The inclusion of two tags was a strategy to allow use of one primer pair to move the expression cassettes across multiple expression vectors using the Getaway technology (Invitrogen) in addition to using the tags for tracking protein expression and affinity purification of recombinant protein. The resultant amino acid sequences of the expression cassettes were used to design synthetic genes codon-optimized for protein expression in swine cells. Codon optimization and gene synthesis was outsourced from GenScript.

Generation of Recombinant Adenovirus.

The synthetic genes encoding the ASFV A151R, B119L, B602L, and B646 antigens were used to generate recombinant adenovirus for immunization. To generate adenovirus constructs, each antigen expression cassette was PCR amplified using flag-specific forward primer containing attB1 sequence and ha-specific reverse primer containing attB2 sequence (Invitrogen), cloned into pDonR-TOPO shuttle vector (Invitrogen), and positive clones were identified by PCR screening and validated by DNA sequencing. Selected recombinant pDonR constructs were then used to transfer cognate antigen expression cassette into the pAd adenovirus backbone (derived from human adenovirus serotype 5) by homologous recombination (Invitrogen). At least six miniprep DNA clones of each pAd construct (namely pAdA151R, pAdB119L, pAdB602L, and pAdB646) were selected based on protein expression as judged by immunocytometric analysis of Human Embryonic Kidney (HEK) 293A cell transfectants probed with anti-FLAG or anti-HA monoclonal antibody. Antigen authenticity was verified by immunocytometric analysis using ASFV-reactive pig serum.

The selected pAd constructs were used to assemble recombinant replication-incompetent adenoviruses by transfecting HEK 293A cells with Pac I-digested miniprep DNA using a well-established protocol (Invitrogen). Six clones of each construct were used to assemble recombinant adenoviruses and the best clones were selected based on protein expression as judged by immunocytometric analysis using anti-tag mAbs and the anti-ASFV pig serum. Each one of the selected clones was amplified in T75 tissue culture flask and used as working stock to generate bulk recombinant adenovirus by infecting 40 T175 flasks for each construct. The bulk viruses, designated AdA151R, AdB119L, AdB602L, and AdB646, were tested for protein expression and antigen authenticity was validated by immunocytometric analysis as above and by Western Blotting. Following titer determination, the presence of replication-competent adenovirus was tested by evaluating replication competence in non-complementing cell lines and primary cells. All the bulk virus preparations were shown to be replication incompetent.

Generation of Recombinant Baculovirus.

The synthetic genes encoding the ASFV A151R, B119L, B602L, and B646 antigens were used to generate recombinant Baculovirus for generation of affinity purified recombinant proteins needed for in vitro evaluation of antibody and T cell responses. Recombinant baculovirus plasmid constructs were similarly generated as above, but flag-specific forward and ha-specific reverse primers were used to PCR each gene. The PCR products were cloned into pFastBac-TOPO shuttle vector (Invitrogen) and positive recombinant pFastBac clones were identified by PCR screening and validated by DNA sequencing. One clone of each pFastBac construct was then used to generate Bacmid plasmid constructs (Invitrogen) encoding each antigen and positive clones were identified by PCR colony screening. At least six clones of each construct were selected based on PCR screening and miniprep DNA was prepared for baculovirus generation.

The Bacmid constructs generated above were transfected into Sf9 insect cells to generate recombinant baculovirus. Positive clones were identified by immunocytometric analysis of the Sf9 insect cell-transfectants probed with the anti-tag mAbs and the anti-ASFV pig serum. Supernatants from the transfected cells were recovered and tested for the presence of baculovirus. Data from the immunocytometric analysis was used to select 3 lead baculovirus clones expressing each antigen. One lead baculovirus for each construct was scaled up in T175 flask, tittered, and frozen in aliquots as working stock. The recombinant Baculoviruses were used for large scale protein expression using the High Five insect cell system (Invitrogen). The expressed proteins were affinity purified from the supernatants of the infected cells using anti-FLAG agarose beads (Sigma), ran on PAGE to evaluate purity, and validated by Western Blot analysis.

### Example 9—Evaluation of the Immunogenicity of Adenovirus-Encoded ASFV Antigens In Vivo

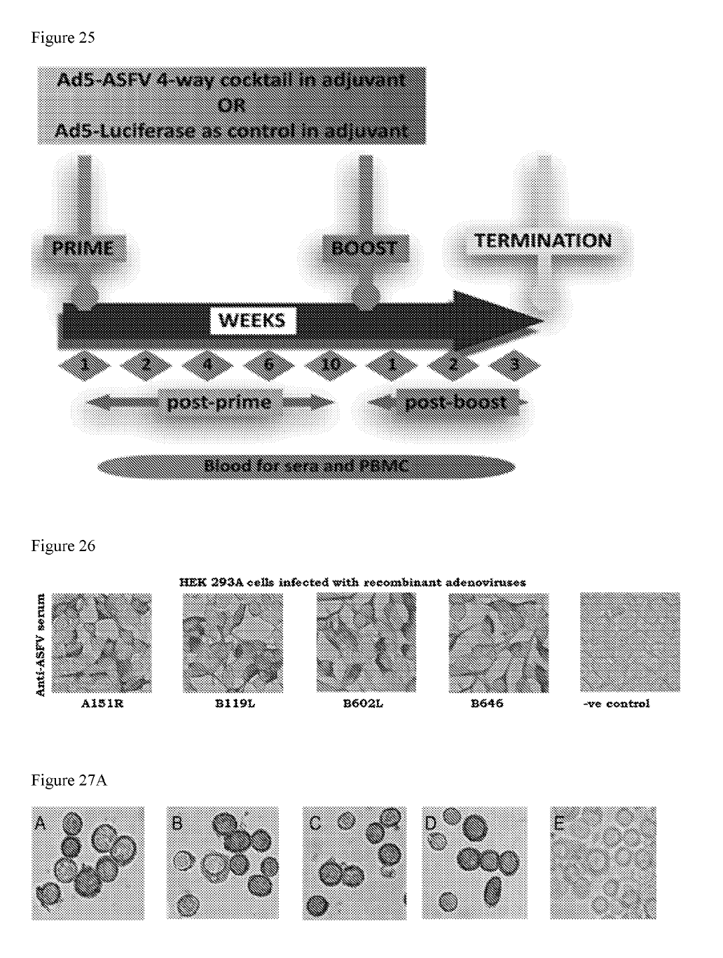

Twenty four weaned piglets (˜30lbs) were acquired and during quarantine period, they were vaccinated against defined pathogens to meet institutional requirements. The piglets were divided into two groups (group 1: n=14 and group 2: n=10) and immunized by inoculation of a cocktail of the AdA151R, AdB119L, AdB602L, and AdB646 recombinant adenoviruses (Table 8). This protocol was also used for boosting (FIG. 25: immunization timeline).

Evaluation of antibody responses: Antigen-specific antibody responses were monitored starting on day 7 post-priming and tracked, biweekly, for ten weeks when the pigs were boosted. Post-boost antibody profiles were monitored, weekly, for 3 weeks and the experiment was terminated (FIG. 25). Antibody responses were evaluated by ELISA using plates coated with the affinity purified recombinant ASFV antigens generated above. Post-prime sera were tested at 1:100 dilutions, whereas post-boost sera were tested at 1:500 dilutions. ELISA was also used to determine antigen-specific endpoint antibody titers. Pre-immunization sera served as the reference normal swine control, whereas ASFV-specific swine sera served as the positive control. The outcomes were presented as mean OD450 nm of triplicate wells of serial sera dilution and an endpoint titer was considered positive if it was 3 standard deviations above the cognate normal swine control. The significance of the differences in mean endpoint titers between the test and the negative control groups were analyzed using Analysis of Variance (ANOVA) followed by Bonferroni post-test analysis (GraphPad Prism Program). A significance level of P<0.05 was used for all analyses.

Recognition of ASF Virus and Native Viral Proteins by Primed Antibodies.

Sera from two weeks post-boost were tested for recognition of the ASF virus by Indirect Immunofluorescence Antibody Assay (IFA) using Vero cells infected with the ASFV George 2007/1 isolate. In addition, the sera were tested for recognition of the native ASFV antigens by Western Blotting using lysates from the ASFV-infected Vero cells.

### Evaluation of IFN-γ Secreting T Cell Responses Post-Prime and Post-Boost.

ELISPOT assays were used to evaluate and quantify antigen-specific IFN-γ-secreting cells in whole peripheral blood mononuclear cells (PBMCs) starting on day 14 post-priming and tracked, biweekly, for ten weeks when the pigs were boosted and post-boost immune profiles were monitored, weekly, for three weeks (FIG. 25). Data from the IFN-γ ELISPOT readouts was presented as Spot Forming Cells (SFC)/million PBMCs. The significance of the differences in mean SFC between the test and the negative control groups was determined by ANOVA followed by Bonferroni post-test analysis as above. A significance level of P<0.05 was used for all analyses.

Evaluation of the Safety and Tolerability of the Proto-Type Vaccine.

Following inoculation of the adenovirus, the piglets were monitored daily by a Veterinarian to determine and document any adverse effects. Inoculation sites were observed for swelling, blebbing/blister formation, ulceration, and granuloma formation. Systemic reactions following inoculation were monitored for general animal demeanor, body temperature, feeding and mobility behavior, depression, and recumbency. Animals were also observed for hypersensitivity by monitoring hyper-salivation, increased nasal discharge, and hyper- or hypo-apnea. In addition, weight change and incidences of diarrhea were monitored.

Recombinant Adenovirus Expressed Encoded Antigens.

Evaluation of protein expression by immunocytometric analysis of adenovirus-infected HEK 293A cells using the anti-ASFV pig serum showed that the assembled replication-incompetent adenoviruses, designated AdA151R, AdB119L, AdB602L, and AdB646, expressed the encoded antigens (FIG. 26). These outcomes were reproducible by immunocytometric analysis of the adenovirus-infected HEK 293A cells using anti-FLAG and the anti-HA mAbs (data not shown). Protein expression by the scaled up adenoviruses was similarly validated by immunocytometric analysis as above and by Western Blotting (data not shown). Virus titers of up to 10¹² ifu (infectious units) were achieved from 40 T175 flasks and all the bulk virus preparations were shown to be replication incompetent.

Baculovirus-Expressed Recombinant Antigens.

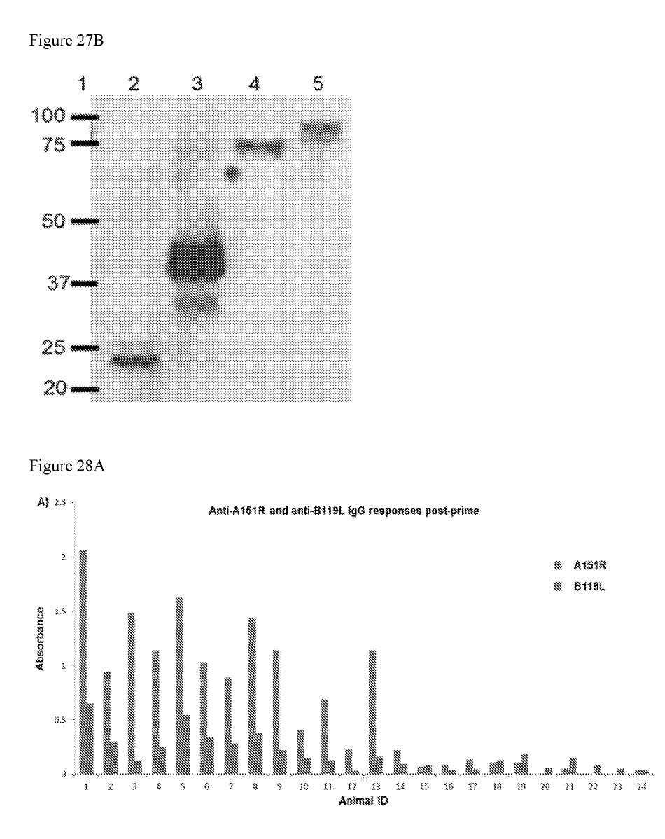

Transfection of Sf9 insect cells with Bacmid constructs encoding A151R, B119L, B602L, or B646 antigens generated cognate recombinant baculovirus and immunocytometric analysis of the Sf9 insect cells infected with the virus using the anti-ASFV pig serum confirmed expression of the ASFV antigens (FIG. 27A). This data was used to select 3 lead baculovirus clones expressing each antigen and one lead baculovirus for each construct was used to generate large scale affinity purified protein (FIG. 27B).

Ad5-ASFv Cocktail Primed ASFV Antigen-Specific Antibodies.

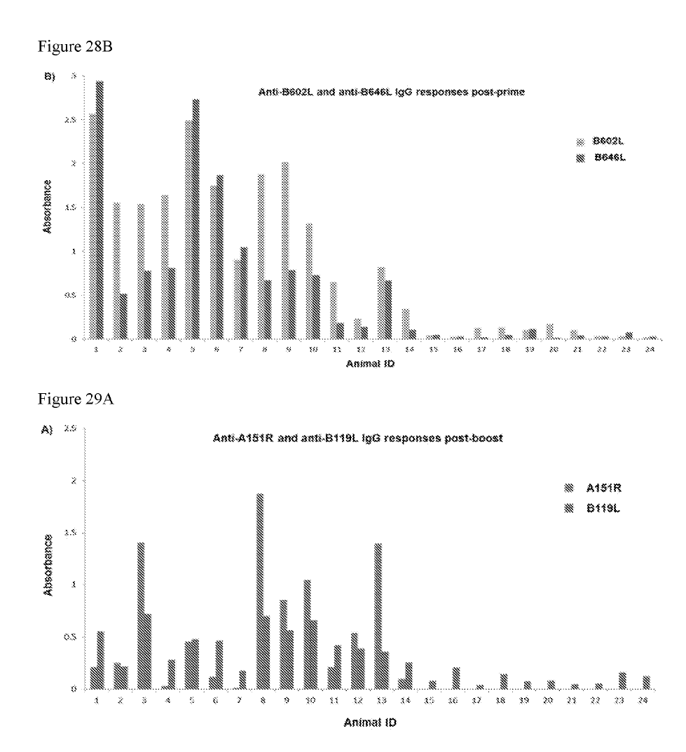

Following priming, ASFV antigen-specific IgG responses were monitored biweekly by ELISA and post-prime sera were tested at 1:100 dilutions. Antibody (IgG) responses specific to the A151R, B119L, B602L, and B646L antigens were detected in all the pigs in the treatment group, but not the sham treatment group (FIGS. 28A-28B). In addition, nearly all the pigs in the treatment group responded well to all the antigens (FIGS. 28A-28B). Data from sera analyzed four weeks post-priming are shown for the pigs in the treatment group (numbers 1-14) and the negative controls (numbers 15-24).

Antigen-Specific IgG Responses Post-Boost.

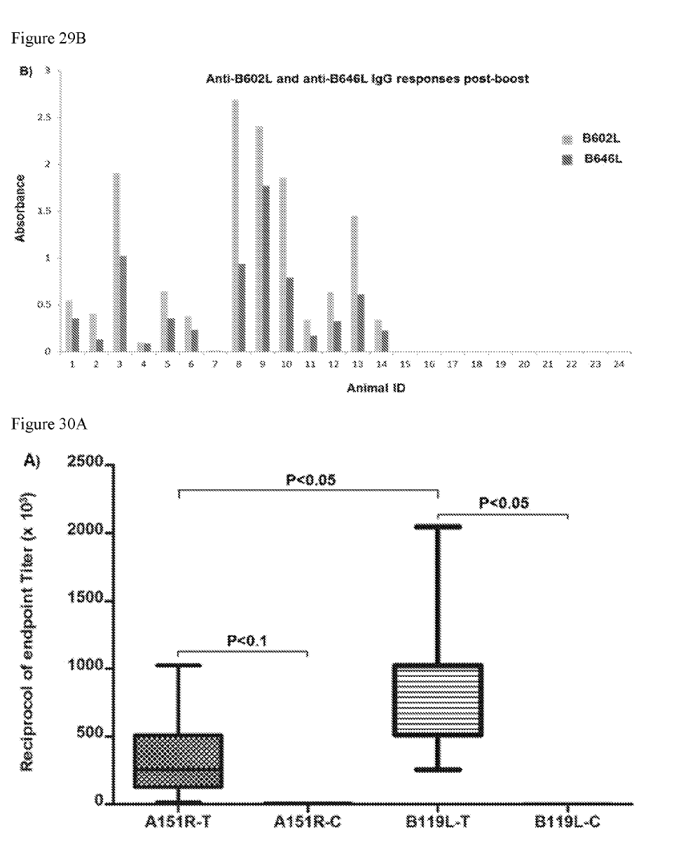

Pigs were boosted 10 weeks post-priming with the cognate priming cocktail and dose (Table 1). Antibody responses were evaluated by ELISA as above and post-boost sera were tested at 1:8,000 dilutions. All the pigs in the treatment group, but not the sham treatment, there was strong recall IgG antibody responses against all the A151R, B119L, B602L, and B646L antigens. Data from sera analyzed two weeks post-boost are shown (FIGS. 29A-29B). Treatment group (numbers 1-14) and the negative controls (numbers 15-24) are shown.

Antigen-Specific IgG Endpoint Titers Post-Boost.

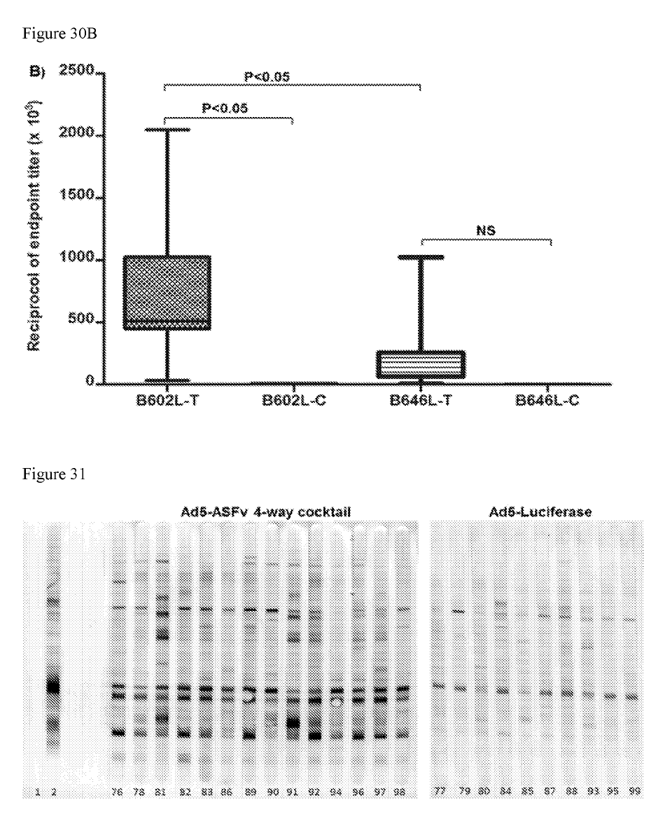

Sera from blood drawn two weeks post-boost were evaluated by ELISA to determine antigen-specific antibody titers. Analysis of the endpoint titers revealed that pigs in the treatment group, compared to the negative controls, had very strong and statistically significant antibody responses against the A151R, B119L, B602L, and B646L antigens (FIGS. 30A-30B). The significance of the difference in mean antibody titer between the treatment and the control groups was determined by ANOVA followed by Bonferroni Multiple Comparison Test. A significance level of P<0.05 was used for all analyses.

Antibodies Primed by the Ad5-ASFv Cocktail Recognize Native ASF Viral Proteins.

The sera from two weeks post-boost were tested by Western blotting at 1:50 dilutions using lysates from Vero cells infected with the ASFV George 2007/1 isolate. Superpig serum (1:10,000) was used as the positive control whereas normal pig serum (1:200) was used as the negative control. Sera from all the pigs immunized with the Ad5-ASFv cocktail, but not the sham treated, strongly recognized the ASFV antigens (FIG. 31). Western blot similarly conducted but using sham-infected Vero cell lysate served as the antigen control.

Antibodies Primed by the Ad5-ASFv Cocktail Recognize ASF Virus.

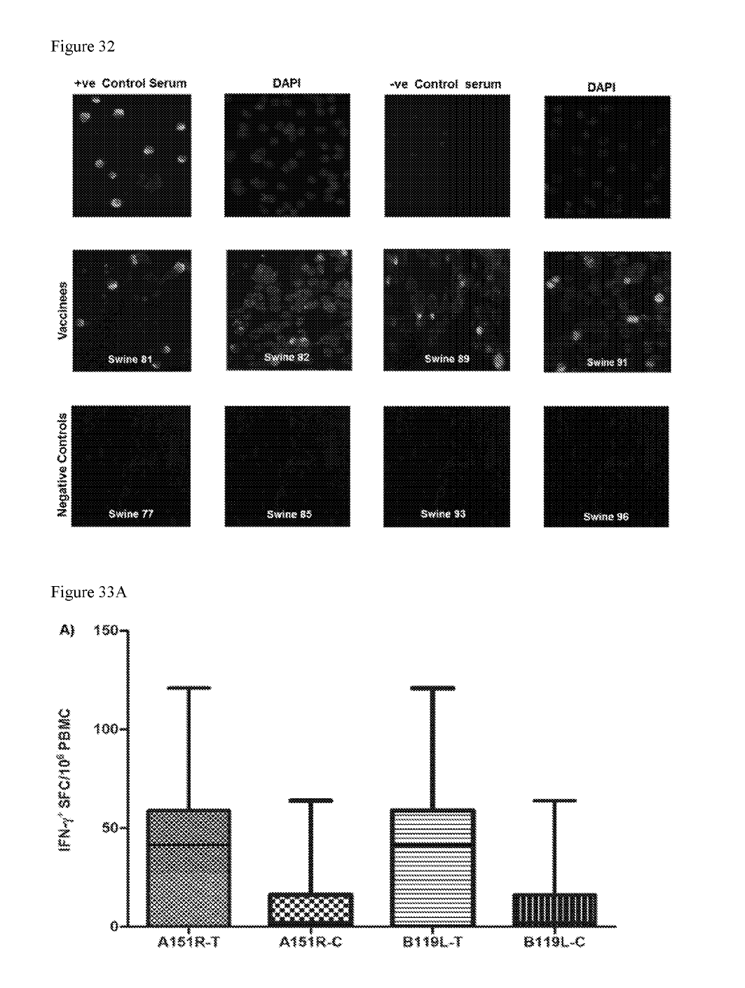

Indirect Immunofluorescence Antibody Assay (IFA) was used to confirm whether antibodies induced by the experimental Ad-ASFv cocktail could recognize the actual ASF virus. The sera from two weeks post-boost were tested by IFA at 1:250 dilutions using Vero cells infected with the ASFV George 2007/1 isolate. Superpig serum (1:10,000) was used as the positive control, whereas normal pig serum (1:250) was used as the negative control. Sera from most of the pigs immunized with the Ad5-ASFv cocktail, but none from the sham treatment group, strongly recognized the ASFV antigens (Table 9 and FIG. 32).

Ad5-ASFv Cocktail Primed IFN-γ-Secreting Cells.

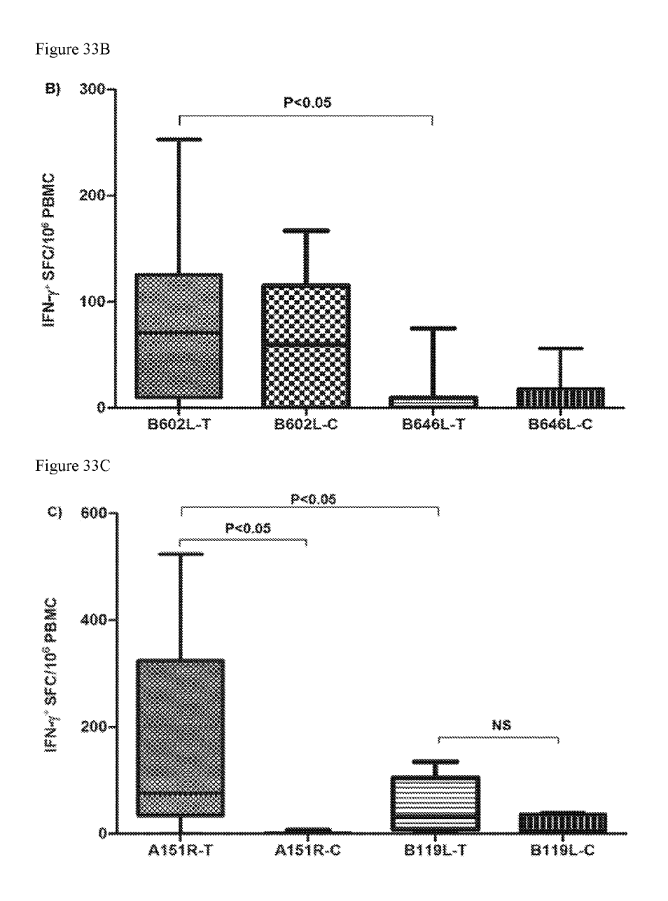

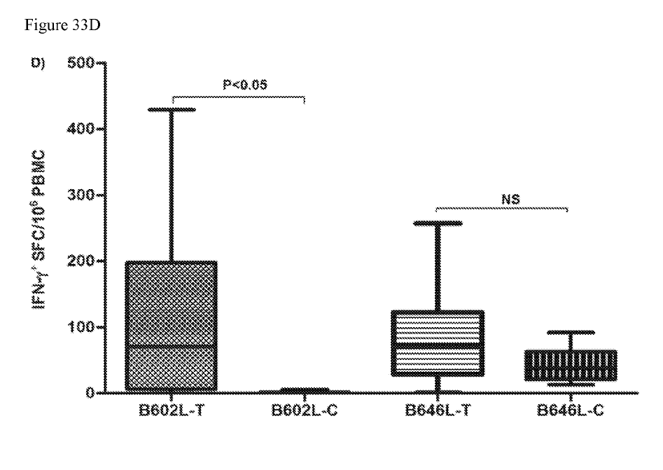

Following immunization of pigs with the Ad5-ASFv cocktail, antigen-specific IFN-γ-secreting cells were detected in whole peripheral blood mononuclear cells (PBMCs) from the vaccinees (FIGS. 33A-33B). Upon boost, there were strong antigen specific recall IFN-γ responses (FIGS. 33C-33D).

Ad5-ASFv Cocktail was Well Tolerated.

Following inoculation of the Ad-ASFV cocktail, the pigs were monitored to document localized and or systemic adverse effects. Three pigs in the test group were observed to be depressed and one had mild fever in the first day after inoculation of the priming rAd5-cocktail dose. However, all the test pigs were normal on all subsequent days. After boosting, one pig in the test group was observed to be depressed and had fever that required treatment. All the pigs in the negative control group were normal post-priming and post-boosting (Table 9).

Summary of Results.

The African Swine Fever Virus (ASFV) poses a high risk to the USA swine industry as it continues to spread globally and since there is no vaccine or treatment, available, a rationally designed live-vectored novel prototype ASFV multi-antigen vaccine was generated and the ability of the vaccine to safely induce immune responses in commercial pigs evaluated. Synthetic genes were used to generate recombinant replication-incompetent adenoviruses designated AdA151R, AdB119L, AdB602L and AdB646L. Protein expression by these recombinant viruses and the authenticity of the expressed antigens was validated using ASFV-specific immune serum from a pig that had been immunized with the ASF virus (FIG. 26). In addition, these genes were used to generate affinity purified recombinant antigens for use in in vitro tests to evaluate and quantify antibody and IFN-γ-secreting T cell responses as readouts for vaccine immunogenicity in pigs. The purified antigens were also shown to be authentic as judged by Western Blot probed with the superpig serum (FIG. 27). This outcome shows that synthetic genes encoding ASFV antigens (a Risk Group 3 pathogen) that require BSL3 biocontainment can safely be used at BSL2 level to develop and test immunogenicity and tolerability of prototype ASFV vaccines.

Immunization of piglets with a cocktail containing the AdA151R, AdB119L, AdB602L and AdB646 (1×10¹¹ IFU/each) induced strong ASFV antigen-specific antibody responses that underwent isotype switching as evidenced by IgG profiles post-priming (FIG. 28). Notably, most of all the pigs in the treatment group mounted strong IgG responses specific to all the antigens in the cocktail (FIG. 28). The primed IgG responses were still detectable ten weeks post-priming when the pigs were boosted. Upon boosting using the priming cocktail and dose, there was strong recall IgG responses against all the antigens in the cocktail suggesting that the priming dose induced antigen-specific memory B cells (FIG. 29). In addition, the mean recall IgG responses in the immunized pigs were significantly higher than the sham-treated pigs and some vaccinees had endpoint titers that reached as high as 1:2×106 for some antigens (FIG. 30). Furthermore, analysis of the post-boost IgG responses showed that a majority of the vaccinees had titers >1:256×103 against the A151R, B119L, and B602L antigens (FIG. 30). Most importantly and relevant to ASFV vaccine development, the induced antibodies strongly recognized the actual ASF viral proteins and ASFV-infected cells as judged by Western Blot and IFA analysis, respectively (Table 9, FIGS. 31 and 32). These outcomes are strong evidence that the strategy tested in this study is a suitable approach for testing immunogenicity of prototype ASFV vaccine candidates. The outcomes also showed that the replication-incompetent adenovirus is an effective vaccine vector and the recall responses post-boost showed that an adenovirus-based ASFV vaccine can be used for homologous prime-boost vaccination and thereby cut costs that could be incurred by use of a heterologous antigen delivery for boosting.

Following priming, analysis of IFN-γ-secreting cells in peripheral blood mononuclear cells (PBMCs) showed that A151R-, B119L-, B602L- and B646L-specific IFN-γ-secreting cells were induced in the vaccinees. However, the responses were not significantly different from the negative controls (FIGS. 33A-33B). Upon boosting, there was a strong recall A151R-specific IFN-γ+ cell response that was significantly (P<0.05) different from the negative controls (FIG. 33C). This response was also significantly (P<0.05) different from the B119L-specific IFN-γ+ cell response (FIG. 33C). However, the B119L-specific IFN-γ+ cell response was not significantly different from the negative controls (FIG. 33C). The B602L-specific IFN-γ+ cell response post-boost was significantly (P<0.05) different from the negative controls, but it was not significantly difference from the B646L-specific response (FIG. 33D). The B646L-specific IFN-γ+ cell response was not significantly different from the negative controls (FIG. 33D). These outcomes showed that immunization of commercial piglets with the adenovirus-vectored experimental vaccine induced ASFV antigen-specific IFN-γ+ cell responses that underwent recall upon boost.

Analysis of the overall performance of the immune responses post-boost showed that, some pigs in the treatment group had strong recall IFN-γ+ cell responses against A151R, B602L, and B646L antigens, whereas some had low responses. This heterogeneity in IFN-γ+ cell response mirrors the outcome observed in regards to recall antibody responses and is expected in an outbred animal population (FIGS. 28-30 and 33). Previous results showed that the natural ASFV chaperones, A151R and B602L, significantly enhanced protein expression in vitro. Whether these chaperones had any effects in vivo on the overall immune responses against the two lead vaccine targets tested, B119L and B646L, was not determined in this study.

Evaluation of local and systemic effects of inoculating the priming and the booster doses of the Ad5-ASFv 4-way cocktail at 1×10¹¹ IFU/each (Table 8) showed that, the prototype vaccine was well tolerated and no serious negative effects were observed. However, mild fever, lack of appetite, and depression were observed in a few piglets on the first day after each inoculation. Thereafter, all the piglets showed normal activity (Table 10). The overall outcome is evidence that a vaccine formulated using a cocktail of replication-incompetent adenovirus expressing protective ASFV antigens is likely to be well tolerated by piglets at doses as high as 10¹¹ IFU used in a prime-boost regimen. This scenario is anticipated since effective ASFV subunit vaccines will likely require delivery of multiple antigens given that studies conducted so far have shown that a combination of one or a few antigens does not confer complete protection.

Taken together, the outcomes from this study showed that the adenovirus-vectored ASFV multi-antigen vaccine cocktail is capable of safely inducing strong antibody and IFN-γ+ cell responses in commercial piglets. These findings support use of the replication-incompetent adenovirus as a vector for the development of a commercial vaccine for protection of pigs against African swine fever virus.

Table 1 List of constructs generated

TABLE 1
List of constructs generated

| | | Target ASFV antigens | | | | | | |
|---|---|---|---|---|---|---|---|---|
| Objective | Item | p32 | p54 | p72 | p62 | p37 | p150-I | p150-II |
| Generation of | Sequence-verified | ✓ | ✓ | ✓ | ✓ | ✓ | ✓ | ✓ |
| genes for the | codon-optimized | | | | | | | |
| target | synthetic genes | | | | | | | |
| antigens | encoding ASFV target | | | | | | | |
| | antigens in pUC57 | | | | | | | |
| Generation of | Multiple clones of | ✓ | ✓ | ✓ | ✓ | ✓ | ✓ | ✓ |
| mammalian | sequence-verified | | | | | | | |
| expression | pCDNA3 constructs | | | | | | | |
| plasmid | encoding target | | | | | | | |
| DNA | antigens | | | | | | | |
| constructs | Multiple clones of the | | | | | | | |
| | pCDNA3 constructs | | | | | | | |
| | expressing the ASFV | | | | | | | |
| | antigens | | | | | | | |
| Generation of | Multiple clones of | ✓ | ✓ | ✓ | ✓ | ✓ | ✓ | ✓ |
| Adenovirus | sequence-verified | | | | | | | |
| constructs | pDonR and respective | | | | | | | |
| | pAd constructs | | | | | | | |
| | Seven lead pAd | | | | | | | |
| | constructs (multiple | | | | | | | |
| | clones) expressing the | | | | | | | |
| | ASFV antigens | | | | | | | |
| | Seven recombinant | | | | | | | |
| | adenovirues (multiple | | | | | | | |
| | clones) expressing the | | | | | | | |
| | ASFV antigens | | | | | | | |
| Generation of | Multiple clones of | ✓ | ✓ | ✓ | ✓ | ✓ | ✓ | ✓ |
| Baculovirus | sequence-verified | | | | | | | |
| constructs | pFastbac and cognate | | | | | | | |
| | Bacmid plasmid | | | | | | | |
| | constructs | | | | | | | |
| | Seven Bacmid | | | | | | | |
| | constructs (multiple | | | | | | | |
| | clones) expressing the | | | | | | | |
| | ASFV antigens | | | | | | | |
| | Seven recombinant | | | | | | | |
| | baculoviruses | | | | | | | |
| | (multiple clones) | | | | | | | |
| | expressing the ASFV | | | | | | | |
| | antigens | | | | | | | |
| Generation of | Multiple clones of | ✓ | ✓ | ✓ | ✓ | ✓ | ✓ | ✓ |
| Lentilvirus | sequence-verified | | | | | | | |
| constructs | pLenti constructs | | | | | | | |
| | Seven pLenti | | | | | | | |
| | constructs (multiple | | | | | | | |
| | clones) expressing the | | | | | | | |
| | ASFV antigens | | | | | | | |
| | Seven recombinant | | | | | | | |
| | Lentiviruses (multiple | | | | | | | |
| | clones) expressing the | | | | | | | |
| | ASFV antigens | | | | | | | |
| | clones) expressing the | | | | | | | |
| | ASFV antigens | | | | | | | |
| Generation of | Multiple clones of | ✓ | ✓ | ✓ | ✓ | ✓ | ✓ | ✓ |
| BacMam | sequence-verified | | | | | | | |
| constructs | pFastbac and | | | | | | | |
| | respective BacMam | | | | | | | |
| | plasmid constructs | | | | | | | |
| | Seven BacMam | | | | | | | |
| | constructs (multiple | | | | | | | |
| | clones) expressing the | | | | | | | |
| | ASFV antigens | | | | | | | |
| | Seven recombinant | | | | | | | |
| | BacMams (multiple | | | | | | | |
| | clones) expressing the | | | | | | | |
| | ASFV antigens | | | | | | | |

Table 2 shows monoclonal antibody reactivity on ASFV- and mock-infected VERO cells

| | Reactivity | Reactivity | |
|---|---|---|---|
| TAMU | to | to Mock | |
| Clone No. | BA71/V | Vero | Comments |
| p62 4G1* | +++ | − | Reactivity identical to original p62 |
| | | | mouse serum, i.e. virus factory |
| | | | positive, tested 2X |
| p62 1C3 | ++ | ++ | Negative specificity, tested 2X |
| p62 2C12 | ++ | ++ | Negative specificity, tested 2X |
| p54 3A9 | +/− | − | Small granules, low affinity or low |
| | | | antibody concentration, tested 2X |
| p54 3C6 | + | − | Small granules and not have virus |
| | | | factory |
| p54 1B7 | + | − | Small granules and not have virus |
| | | | factory |
| p54 2B1 | + | − | Small granules and not have virus |
| | | | factory |
| p54 2G12 | + | − | Small granules and not have virus |
| | | | factory |
| p54 1H10 | + | − | Small granules and not have virus |
| | | | factory |
| p54 1A2 | ++ | ++ | Negative specificity |
| p54 3A5 | +/− | − | Small granules, low affinity or low |
| | | | antibody concentration |
| p62 2B2 | + | − | Small granules and not have virus |
| | | | factory |
| p62 4B4 | ++ | − | Small granules and not have virus |
| | | | factory |
| p62 1A3 | + | − | Small granules and not have virus |
| | | | factory |
| p62 1B1 | ++ | ++ | Negative specificity |
| p62 2E1 | + | − | Uniform cytoplasm fever granules |
| p62 1C9 | + | − | Small granules and not have virus |
| | | | factory |
| p62 4H11 | + | − | Small granules and not have virus |
| | | | factory |
| p62 1A2 | + | − | Small granules and not have virus |
| | | | factory |
| p62 3F3 | +/− | +/− | Cytoplasm = F14 |
| p62 3F2 | +++ | − | Speckles |
| p54 2B3 | +/− | +/− | Cytoplasm = F14 |
| p54 1B9 | +/− | − | Cytoplasm = F14 |
| p54 1B4 | +/− | +/− | Cytoplasm = F14 |
| p54 2B5 | +++ | − | Cytoplasm very bright |
| p54 2A10 | ++ | − | Membranes |
| p54 3B12 | +/− | +/− | Cytoplasm = F14 |
| p54 2B7 | ++ | − | Speckles |
| p62 4G1* | +/− | +/− | |
| p62 1F12 | ++ | − | Virus factory |
| p54 3D5 | +/− | +/− | |
| p54 1A2 | +++ | − | Membrane |
| p62 4B4 | +/− | +/− | |
| p62 4H11 | +/− | +/− | |
| p62 1A2 | +/− | +/− | |
| p62 1E6 | +/− | +/− | |
| p54 Mouse | +++++ | − | Virus factory |
| serum | | | |
| P62 Mouse | ++++ | − | Virus factory + cytoplasm |
| serum | | | |
| F14 FMDV | +/− | +/− | |
| 2nd alone | − | − | |

Table 3 shows hybridomas selected for subcloning

| | Reactivity | | |
|---|---|---|---|
| | on | Reactivity | |
| | Georgia- | on BA71- | |
| | Infected | Infected | |
| | Macs | Macs | Comments |
| Anti-p62 1F12 | + | + | Virus factory with Georgia Macs |
| Anti-p62 3F2 | +++ | NA | 38% cell reactivity |
| Anti-p62 4G1 | ++ | +++ | Virus factory with |
| | | | BA71/V Macs |
| Anti-p62 4B4 | ++ | ++ | Uniform imaged, speckles, small |
| | | | granules |
| Anti-p54 2B7 | ++ | NA | 19% cell reactivity |
| Anti-p54 3D5 | ++ | ++ | Very large speckles |
| Anti-p54 2A10 | ++ | NA | 48% cell reactivity, “too |
| | | | numerous to be specific” |
| Anti-p54 2B5 | +++ | NA | 100% cell reactivity, cytoplasm |
| | | | very bright |
| Anti-p54 2E1 | + | + | Speckles |

Table 4 Rabbit polyclonal and mouse monclonal antibodies

TABLE 4
Rabbit polyclonal and mouse monclonal antibodies

| Antigen | Antibody | Status |
|---|---|---|
| p54 | Rabbit anti-p54 polyclonal | Antibodies and antibody |
| | Mouse anti-p54 monoclonal | producing hybridomas |
| | | produced |
| p62 | Rabbit anti-p62 polyclonal | Antibodies and antibody |
| | Mouse anti-p62 monoclonal | producing hybridomas |
| | | produced |

Table 5 shows the immunization protocol

| Treatment | No. of | | | |
|---|---|---|---|---|
| Groups | pigs | Immunogen | Dose per pig | Adjuvant |
| T01 | 5 | Ad5-ASFv 7-way | 7 × 10¹⁰ IFU* | ENABL**** |
| | | cocktail | | |
| T02 | 5 | Ad5-ASFv 7-way | 7 × 10¹¹ IFU** | ENABL**** |
| | | cocktail | | |
| T03 | 5 | Ad5-Luciferase | 7 × 10¹¹ IFU*** | ENABL**** |
| T04 | 5 | Ad5-ASFv 7-way | 7 × 10¹¹ IFU** | Zoetis***** |
| | | cocktail | | |

*ASFv 7-way cocktail: pool of 7 Ad5-ASFv constructs each 1 × 10¹⁰ IFU
**ASFv 7-way cocktail: pool of 7 Ad5-ASFv constructs each 1 × 10¹¹ IFU
***Ad5-Luciferase Sham control
****ENABL Adjuvant (Cat # 7010106-C6)
*****Experimental Adjuvant

Table 6 shows the summary of immunogen safety and tolerability

| | | | Reaction | |
|---|---|---|---|---|
| Group | Animal # | Treatment | Post-prime | Post-boost |
| 1 | 33, 35, | 1 × 10¹⁰ IFU rAd- | No | Day 1: All animals depressed. |
| | 36, 40, 42 | ASFV; ENABL | reaction | #s 33 and 40 had swelling at the |
| | | adjuvant | | injection site on neck. #40 also |
| | | | | had fever and was recumbent |
| | | | | Day 2: All animals were |
| | | | | depressed with inappetence. |
| | | | | Except for #s 33 and 42, all |
| | | | | others received IM Banamine |
| | | | | Day 3: Normal Activity with |
| | | | | good appetite |
| 2 | 34, 41, | 1 × 10¹¹ IFU rAd- | No | Day 1: All animals depressed |
| | 43, 46, 48 | ASFV; ENABL | reaction | with inappetence. #s 34 and 48 |
| | | adjuvant | | had rreddish large swelling at the |
| | | | | injection site |
| | | | | Day 2: All animals were |
| | | | | depressed with reduced appetite |
| | | | | Day 3: Normal activity with |
| | | | | good appetite |
| 3 | 32, 38, | 7 × 10¹¹ IFU rAd- | No | Day 1: All animals depressed |
| | 39, 44, 45 | Luciferase; ENABL | reaction | with inappetence. #s 39, 45 and |
| | | adjuvant | | 44 had swollen injection site |
| | | | | Day 2: All animals were |
| | | | | depressed with inappetence. |
| | | | | Except for #s 39 and 44, all |
| | | | | others received IM Banamine |
| | | | | Day 3: Normal activity with |
| | | | | good appetite |
| 4 | 31, 37, | 1 × 10¹¹ IFU rAd- | No | Day 1: All animals showed |
| | 93, 94, 96 | ASFV; Experimental | reaction | normal activity. All had a pink |
| | | Zoetis adjuvant | | spot at the site of injection on the |
| | | | | neck |
| | | | | Day 2: All animals were active |
| | | | | Day 3: Normal activity with |
| | | | | good appetite |

Table 7 shows IFA reactivity of swine anti-ASFV sera

| | Reactivity | | | Reactivity | |
|---|---|---|---|---|---|
| | ASFV- | Mock- | | ASFV- | Mock- |
| Group 1: Pig | infected | Infected | Group 1: Pig | infected | Infected |
| No. | macrophage | macrophage | No. | macrophage | macrophage |
| 33 | ++ | Negative | 32 | Negative | Negative |
| 35 | +++ | Negative | 38 | Negative | Negative |
| 36 | +++ | Negative | 39 | Negative | Negative |
| 40 | ++ | Negative | 44 | Negative | Negative |
| 42 | ++ | Negative | 45 | Negative | Negative |
| Group 2: Pig | | | Group 4: Pig | | |
| No: | | | No: | | |
| 34 | +++ | Negative | 31 | +++ | Negative |
| 41 | ++ | Negative | 37 | ++ | Negative |
| 43 | ++++ | Negative | 93 | ++++ Best | Negative |
| 46 | +++ | Negative | 94 | +++ | Negative |
| 48 | +++ | Negative | 96 | +++ | Negative |
| Superpig serum | ++++ | Negative | Normal Serum | Negative | Negative |

Table 8 shows the immunization protocol of Ad5-ASFv 4-way cocktail (AdA151R, AdB119L, AdB602L and AdB646L) vaccinated pigs

| Treatment Groups | No. of pigs | Immunogen | Dose per pig |
|---|---|---|---|
| T01 | 14 | AS5-ASFv 4-way | 4 × 10¹¹ IFU* |
| | | cocktail | |
| T02 | 10 | Ad5-Luciferase | 4 × 10¹¹ IFU** |

*Ad5-ASFv 4-way cocktail = AdA151R, AdB119L, AdB602L and AdB646L each at 1 × 10¹¹ IFU
**Ad5-Luciferase Sham control at 4 × 10¹¹ IFU total

Table 9 shows the reactivity of primed antibodies recgnizing ASF virus

| | Reactivity | | | Reactivity | |
|---|---|---|---|---|---|
| | ASFV- | Mock | | ASFV- | Mock |
| Treatment | infected | infected | Control | infected | infected |
| Group: | Vero | Vero | group: | Vero | Vero |
| Pig No. | cells | cells | Pig No. | cells | cells |
| 76 | Negative | Negative | 77 | Negative | Negative |
| 78 | Negative | Negative | 79 | Negative | Negative |
| 81 | ++ | Negative | 80 | Negative | Negative |
| 82 | +++ | Negative | 84 | Negative | Negative |
| 83 | Negative | Negative | 85 | Negative | Negative |
| 86 | + | Negative | 87 | Negative | Negative |
| 89 | +++ | Negative | 88 | Negative | Negative |
| 90 | + | Negative | 93 | Negative | Negative |
| 91 | ++++ | Negative | 95 | Negative | Negative |
| 92 | + | Negative | 99 | Negative | Negative |
| 94 | ++ | Negative | 94 | Negative | Negative |
| 96 | +++ | Negative | 96 | Negative | Negative |
| 97 | +++ | Negative | | | |
| 98 | ND | ND | | | |
| Superpig | ++++ | Negative | Normal | Negative | Negative |
| serum | | | Serum | | |

Table 10 shows the summary of vaccine safety and tolerabiiity data in Ad5-ASFV 4-way cocktail vaccinated pigs.

| | | | Reaction | |
|---|---|---|---|---|
| Group | Animal # | Treatment | Post-prime | Post-boost |
| 1 | 76, 78, | 1 × 10¹¹ IFU | Day 1: Three (#s 78, | Day 1: one pig (#96) |
| | 81, 82, | rAd5-cocktail | 84, 87) of 14 pigs were | was depressed with |
| | 83, 86, | adjuvant | depressed with | inappetence and fever; |
| | 89, 90, | | inappetence; #84 also | received NSAID only. |
| | 91, 92, | | had fever | |
| | 94, 96, | | Subsequent days: All | Subsequent days: All |
| | 97, 98 | | animals showed normal | animals showed normal |
| | | | activity with good | activity with good |
| | | | appetite | appetite |
| 2 | 77, 79 | 1 × 10¹¹ IFU | No reaction | Normal activity with |
| | 80, 84 | rAd5-Luciferase | | good appetite |
| | 85, 87 | adjuvant | | |
| | 88, 93 | | | |
| | 95, 99 | | | |

```
A151R-
SEQ ID NO: 15 and SEQ ID NO: 16
atgggggactacaaggacgatgacgataagaaaatgaacaagaagattatcgtgatgatg
  M G D Y K D D D D K K M N K K I I V M M
gctctgctgcacaaagaaaaactgattgagtgtattgaaaatgaactggaaaacggaggc
  A L L H K E K L I E C I E N E L E N G G
accgtgctgctcctgacaaagaacatcgtggtctctgagatcagctacattggcaatacc
  T V L L L T K N I V V S E I S Y I G N T
tacaaatatttcaccttcaacgacaatcacgatctcatctccaaggaagacctgaaaggg
  Y K Y F T F N D N H D L I S K E D L K G
gccacctctaacaatatcgctaagatgatctacaactggatcattaagaatccccagaac
  A T S N N I A K M I Y N W I I K N P Q N
aacaaaatctggagcggagagcctcgcacccaaatctacttcgaaaacgacctctaccac
  N K I W S G E P R T Q I Y F E N D L Y H
acaaactacaaccatgagtgcatcaaagatttctggaacgtgagcacctccgtcggcccc
  T N Y N H E C I K D F W N V S T S V G P
tgcatctttaacgatcggtccatttggtgtacaaagtgtacctccttttatccttttacc
  C I F N D R S I W C T K C T S F Y P F T
aacattatgagccccaacattttccagaaaaaatacccatacgacgttccggactacgct
  N I M S P N I F Q K K Y P Y D V P D Y A
tcttagtgataa
  S - - -
B119L (9GL)-
SEQ ID NO: 17 and SEQ ID NO: 18
atgggggactacaaggacgatgacgataagaaaatgctccactgggggcctaaatactgg
  M G D Y K D D D D K K M L H W G P K Y W
cggtccctgcacctctacgctatcttcttctctgatgctccctcatggaaggaaaaatac
  R S L H L Y A I F F S D A P S W K E K Y
gaggccatccagtggattctgaacttcatcgaatccctcccctgcacccgctgtcagcac
  E A I Q W I L N F I E S L P C T R C Q H
catgcttttagctacctgaccaagaacccactgacactcaacaattctgaggacttccag
  H A F S Y L T K N P L T L N N S E D F Q
tattggacattcgcctttcacaacaatgtgaacaatcggctgaacaagaaaatcatctct
  Y W T F A F H N N V N N R L N K K I I S
tggtcagagtacaagaacatctatgaacagagcatcctgaagaccattgaatacggcaaa
  W S E Y K N I Y E Q S I L K T I E Y G K
acagattttattggagcttggtccagcctcaaaaaatacccatacgacgttccggactac
  T D F I G A W S S L K K Y P Y D V P D Y
gcttcttagtgataa
  A S - - -
B602L (92L)-
SEQ ID NO: 19 and SEQ ID NO: 20
atgggggactacaaggacgatgacgataagaaagccgaattcaatatcgacgaactcctc
  M G D Y K D D D D K K A E F N I D E L L
aaaaatgtcctggaagacccaagcacagagattagcgaagagaccctcaagcagctctac
  K N V L E D P S T E I S E E T L K Q L Y
cagcgcaccaacccctataagcagttcaaaaatgactccagggtggccttctgctctttt
  Q R T N P Y K Q F K N D S R V A F C S F
ccaacctcagagagcagtacatccgccggctgattatgaccagcttcatcggctatgtg
  T N L R E Q Y I R R L I M T S F I G Y V
tttaaggccctgcaggagtggatgccatcctactctaagcccacacacaccacaaaaacc
  F K A L Q E W M P S Y S K P T H T T K T
ctgctctccgagctgatcaccctcgtggacacactgaagcaggaaacaaacgatgtccct
  L L S E L I T L V D T L K Q E T N D V P
agcgagtccgtggtcaataccatcctgtccattgctgactcttgtaagacccagacacag
  S E S V V N T I L S I A D S C K T Q T Q
aagagcaaagaagccaaaaccacaatcgactccttcctgagggagcacttcgtgtttgat
  K S K E A K T T I D S F L R E H F V F D
cccaacctgcatgctcagagcgcctacacctgcgcttccacaaacgccgacacctctgct
  P N L H A Q S A Y T C A S T N A D T S A
agcacaaatgtggacacctgcgtcgatacatgtgccagcatgggagcttccacctgtgcc
  S T N V D T C V D T C A S M G A S T C A
gacacaaatgtggatacctgcgcttctatggatacctgtgccagcaccgaatatacagac
  D T N V D T C A S M D T C A S T E Y T D
ctcgccgatcccgagcgcatccccctgcacattatgcagaagaccctgaacgtgcccaat
  L A D P E R I P L H I M Q K T L N V P N
gagctccaggctgacatcgatgccattacccagacacctcaggggtacagagccgctgcc
  E L Q A D I D A I T Q T P Q G Y R A A A
catatcctgcagaacattgaactccaccagagcatcaagcatatgctggagaaccctcgc
  H I L Q N I E L H Q S I K H M L E N P R
gccttcaagccaatcctctttaataccaaaattacacggtacctgtcccagcacatcccc
  A F K P I L F N T K I T R Y L S Q H I P
cctcaggacaccttctacaagtggaactactacatcgaggataactacgaggaactgagg
  P Q D T F Y K W N Y Y I E D N Y E E L R
gctgccaccgagagcatctatccagaaaagcccgacctggagttcgcctttatcatctac
  A A T E S I Y P E K P D L E F A F I I Y
gacgtggtcgatagctccaaccagcagaaggtggacgaattctactacaagtacaaggat
  D V V D S S N Q Q K V D E F Y Y K Y K D
cagattttcagcgaggtctctagcattcagctggggaactggaccctgctcggcagcttc
  Q I F S E V S S I Q L G N W T L L G S F
aaggccaacagggaaagatacaactacttcaaccagaacaacgagatcatcaagcgcatc
  K A N R E R Y N Y F N Q N N E I I K R I
ctggaccggcacgaggaggacctgaagatcggcaaagaaattctgagaaacaccatctat
  L D R H E E D L K I G K E I L R N T I Y
cataagaaggctaagaacatccaggagaccggacctgacgctccaggactgtctatctac
  H K K A K N I Q E T G P D A P G L S I Y
aacagcaccttccacacagattccggcattaaggggctgctctcttttaaggaactgaaa
  N S T F H T D S G I K G L L S F K E L K
aacctcgagaaggccagcgggaatatcaagaaagcccgcgagtacgacttcatcgacgat
  N L E K A S G N I K K A R E Y D F I D D
tgcgaggaaaagatcaagcagctgctctctaaggaaaacctgacaccagacgaggaatcc
  C E E K I K Q L L S K E N L T P D E E S
gagctcatcaaaaccaagaaacagctggataacgccctggagatgctcaatgtgcccgac
  E L I K T K K Q L D N A L E M L N V P D
gataccatccgggtcgacatgtgggtgaataacaacaacaaactggagaaagaaatcctc
  D T I R V D M W V N N N N K L E K E I L
tacaccaaagccgaactcaaaaaatacccatacgacgttcoggactacgottottagtga
  Y T K A E L K K Y P Y D V P D Y A S - -
taa
  -
B646L (p72) -
SEQ ID NO: 21 and SEQ ID NO: 22
atggactacaaggacgatgacgataaggcagggccaggacctggcccctcagcttctggg
  M D Y K D D D D K A G P G P G P S A S G
ggggctttctgtctcatcgctaacgacggcaaggctgataaaatcattctcgctcaggac
  G A F C L I A N D G K A D K I I L A Q D
ctcctcaactctcggatctccaacattaagaatgtgaacaaatcttacggcaagccagac
  L L N S R I S N I K N V N K S Y G K P D
cccgaacctaccctgagccagatcgaggaaacacacctcgtgcatttcaacgcccatttt
  P E P T L S Q I E E T H L V H F N A H F
aaaccatacgtgcccgtcgggttcgagtataacaaggtgcgcccacacaccggcacaccc
  K P Y V P V G F E Y N K V R P H T G T P
accctcgggaataagctgacctttggaatcccacagtacggcgacttctttcatgatatg
  T L G N K L T F G I P Q Y G D F F H D M
gtggggcaccatatcctgggagcttgccacagctcctggcaggacgctccaatccagggc
  V G H H I L G A C H S S W Q D A P I Q G
accagccagatgggagctcacggacagctgcagacattccctcggaacgggtacgactgg
  T S Q M G A H G Q L Q T F P R N G Y D W
gataatcagaccccactggaaggagccgtgtatacactcgtcgatcccttcggaaggcct
  D N Q T P L E G A V Y T L V D P F G R P
atcgtgccaggcaccaagaacgcttacagaaatctggtctactattgcgagtaccccgga
  I V P G T K N A Y R N L V Y Y C E Y P G
gaaaggctctatgagaacgtgagattcgacgtgaatggcaactccctggacgagtactct
  E R L Y E N V R F D V N G N S L D E Y S
agcgatgtgaccacactcgtccgcaagttttgtatccccggcgataaaatgaccgggtat
  S D V T T L V R K F C I P G D K M T G Y
aagcacctggtgggccaggaagtgtctgtcgaggggaccagcggacctctgctctgcaac
  K H L V G Q E V S V E G T S G P L L C N
attcacgacctgcataaacctcaccagagcaagccaatcctcaccgacgaaaacgataca
  I H D L H K P H Q S K P I L T D E N D T
cagcggacctgttcccacacaaatcctaaattcctgtctcagcattttccagagaacagc
  Q R T C S H T N P K F L S Q H F P E N S
cacaatatccagaccgccgggaagcaggacatcacacccattaccgacgctacatacctg
  H N I Q T A G K Q D I T P I T D A T Y L
gacatccgccggaacgtgcactatagctgtaatggaccocagacccctaaatactatcag
  D I R R N V H Y S C N G P Q T P K Y Y Q
ccccctctcgccctgtggatcaagctgcgcttctggtttaatgaaaacgtgaatctcgct
  P P L A L W I K L R F W F N E N V N L A
atcccctctgtcagcattcctttcggcgagcggtttatcaccatcaagctggcctcccag
  I P S V S I P F G E R F I T I K L A S Q
aaggacctcgtgaacgagttccccggcctgtttatcaggcagagccggttcattccaggg
  K D L V N E F P G L F I R Q S R F I P G
cgcccctccaggagaaacatccggttcaagccctggtttatccccggcgtgatcaacgaa
  R P S R R N I R F K P W F I P G V I N E
attagcctcaccaacaatgagctgtacatcaacaatctcttcgtgacacctgagattcac
  I S L T N N E L Y I N N L F V T P E I H
aacctgttcgtgaaacgcgtccggttttccctcatcagggtgcataagacccaggtcaca
  N L F V K R V R F S L I R V H K T Q V T
cacaccaacaataaccaccatgacgaaaaactcatgtctgccctgaagtggcccatcgag
  H T N N N H H D E K L M S A L K W P I E
tatatgttcattggcctgaaacccacctggaacatctccgaccagaatcctcaccagcat
  Y M F I G L K P T W N I S D Q N P H Q H
agggattggcataagttcgggcacgtggtcaacgccatcatgcagcctacccaccatgct
  R D W H K F G H V V N A I M Q P T H H A
gagatttcctttcaggacagagatacagccctgccagacgcttgctcctctatcagcgat
  E I S F Q D R D T A L P D A C S S I S D
atttccccagtgacctaccccatcacactgcctatcattaagaacatttccgtcaccgcc
  I S P V T Y P I T L P I I K N I S V T A
cacggcatcaatctgattgacaaattcccatctaagttttgtagctcctacatccccttc
  H G I N L I D K F P S K F C S S Y I P F
cactatggcgggaacgccatcaagacccctgacgatccaggggccatgatgatcacattt
  H Y G G N A I K T P D D P G A M M I I T
gctctgaagccaagggaggaataccagccctccggacacatcaacgtgtctagggccaga
  A L K P R E E Y Q P S G H I N V S R A R
gagttctacatttcttgggacaccgattatgtcggaagcatcaccacagctgacctggtg
  E F Y I S W D T D Y V G S I T T A D L V
gtctccgcctctgctatcaacttcctgctcctgcagaatggcagcgccgtgctgagatac
  V S A S A I N F L L L Q N G S A V L R Y
tccacctacccatacgatgttccagattacgcttga
  S T Y P Y D V P D Y A -
```

### REFERENCES

1. Brito et al. (2013), Vaccine adjuvant formulations: A pharmaceutical perspective, Seminars in Immunology, 25:130-145.

1-22. (canceled)

23. A multivalent vaccine comprising an immunologically effective amount of one or more recombinant virus(es) expressing one or more antigenic protein or antigenic fragment thereof, wherein the vaccine induces, in a vaccinee, a humoral and cellular immune response to multiple antigenic proteins or antigenic fragments thereof expressed by the recombinant virus(es) of the cocktail, provided that where the vaccine comprises a single recombinant virus, the recombinant virus expresses multiple antigenic proteins or antigenic fragments thereof and where the vaccine comprises a cocktail of recombinant viruses, each virus of said cocktail expresses one or more antigenic protein or antigenic fragment thereof.

24. The vaccine according to claim 23, additionally comprising a pharmaceutically acceptable carrier and/or an adjuvant.

25. The vaccine according to claim 23, wherein the vaccine comprises one or more recombinant adenovirus(es), one or more replication-incompetent recombinant adenovirus(es), one or more replication-incompetent recombinant lentivirus(es), or a combination of one or more replication-incompetent recombinant adenovirus(es) and lentivirus(es).

26. The vaccine according to claim 23, wherein the antigenic protein or fragment thereof is derived from a pathogen that is relatively impervious to vaccine development.

27. The vaccine according to claim 26, wherein the antigenic protein or fragment thereof is derived from African Swine Fever Virus (ASFV).

28. The vaccine according to claim 27, wherein the one or more antigenic protein or fragment thereof is any of SEQ ID NOs: 2, 4, 6, 8, 10, 12, 14, 16 or 18.

29. The vaccine according to claim 23, wherein the said one or more virus(es) contain one or more codon-optimized gene(s) for expression of the antigenic protein or fragment thereof in the vaccinee.

30. The vaccine according to claim 29, wherein the vaccine comprises a viral cocktail is of recombinant viruses each carrying a codon-optimized gene for efficient expression of an ASFV antigenic protein or fragment thereof in the vaccinee.

31. The vaccine according to claim 29, wherein the vaccinee is a human or non-human mammal.

32. The vaccine according to claim 31, wherein the vaccinee is a human.

33. The vaccine according to claim 31, wherein the vaccinee is a pig.

34. The vaccine according to claim 23, wherein the antigenic proteins or fragment thereof is expressed as a fusion protein.

35. The vaccine according to claim 34, wherein the fusion protein targets the protein or fragment thereof to a target tissue, organ or cell and wherein the fusion protein comprises an antibody, a fragment of antibody or a biomolecule, wherein the antibody or the biomolecule specifically binds to one or more surface biomolecules present on the target tissue, organ or cell.

36. The vaccine according to claim 35, wherein the surface biomolecule is Fe receptor, C-type lectin, complement receptor, major histocompatibility protein, or a receptor present on the surface of dendritic cells or antigen presenting cells.

37. The vaccine according to claim 34, wherein the protein or fragment thereof is fused to a heterologous protein or peptide.

38. The vaccine according to claim 37, wherein the heterologous protein is dendritic cell targeting peptide (DC-pep), ovalbumin or bovine serum albumin.

39. A method of immunizing a subject against an infection by a pathogen that is relatively impervious to vaccine development, the method comprising administering to the subject a vaccine of claim 23.

40. The method according to claim 39, wherein the vaccine is administered via subcutaneous, intradermal, intranasal, oral, intramuscular, intraperitoneal, or other parenteral or enteral route.

41. The method according to claim 39, wherein the vaccine is administered as a single dose or multiple doses.
